(19) United States
(12) Patent Application Publication
(10) Pub. No.: US 2023/0220379 A1
(43) Pub. Date: Jul. 13, 2023

(54) HIGH PURITY gRNA SYNTHESIS PROCESS

(71) Applicant: CRISPR Therapeutics AG, Zug (CH)

(72) Inventors: Rajat Das, Cambridge, MA (US); Ganapathy Sankaran, Cambridge, MA (US)

(21) Appl. No.: 17/934,799

(22) Filed: Sep. 23, 2022

Related U.S. Application Data
(60) Provisional application No. 63/248,102, filed on Sep. 24, 2021.
(60) Provisional application No. 63/293,999, filed on Dec. 27, 2021.

(51) Int. Cl. C12N 15/11; C12N 9/22; C12N 9/00

(52) U.S. Cl. CPC C12N 15/11; C12N 9/22; C12N 9/93; C12N 2310/20; C12Y 605/01003

(57) ABSTRACT

The present disclosure relates to methods, compositions and kits for synthesizing moderate length RNAs (mlRNAs, including gRNAs) by splint-mediated ligation of RNA fragments. The synthesis of moderate length RNAs can be followed by DNase treatment. In some embodiments, splint DNA oligonucleotides that are no longer than 32 nucleotides are used.

### CROSS-REFERENCE TO RELATED APPLICATIONS

This application claims the benefit under 35 U.S.C. § 119(e) to U.S. Provisional Patent Application No. 63/248,102 filed on Sep. 24, 2021, and U.S. Provisional Patent Application No. 63/293,999 filed on Dec. 27, 2021; the content of each of which is incorporated herein by reference in its entirety for all purposes.

### REFERENCE TO SEQUENCE LISTING

The present application is being filed along with a Sequence Listing in electronic format. The Sequence Listing is provided as a file entitled Sequence_Listing_80EM-341697-US, created Sep. 12, 2022, which is 121 kilobytes in size. The information in the electronic format of the Sequence Listing is incorporated herein by reference in its entirety

### BACKGROUND

### Field

The present disclosure generally relates to the field of molecular biology and biotechnology, including the synthesis of RNA molecules.

### Description of the Related Art

The targeting of DNA using the RNA-guided, DNA-targeting principle of CRISPR (Clustered Regularly Interspaced Short Palindromic Repeats)-Cas (CRISPR associated) systems has been widely used. CRISPR-Cas systems can be divided in two classes, with class 1 systems utilizing a complex of multiple Cas proteins (such as type I, III, and IV CRISPR-Cas systems) and class 2 systems utilizing a single Cas protein (such as type II, V, and VI CRISPR-Cas systems). Type II CRISPR-Cas-based systems have been used for genome editing, and require a Cas polypeptide or variant thereof guided by a customizable guide RNA (gRNA) for programmable DNA targeting.

Available approaches for the synthesis of gRNA include intracellular transcription of an exogenous plasmid and solid-phase synthesis using phosphoramidite chemistry. Direct chemical synthesis of gRNA allows for incorporation of chemical modifications that increase the chemical stability of the RNA, decrease its immunogenicity, and reduce potential off-target effects (i.e., cleaving genomic DNA at undesired locations). Chemical synthesis is not optimal for gRNAs which are fairly long RNAs typically ranging from 60 to 100 nucleotides. For example, if the phosphoramidite chemistry being used has a coupling efficiency of ˜0.99^{X} (where X is the number of nucleotides), the overall synthesis process would be expected to yield approximately 30-40% full-length product (FLP) when synthesizing gRNAs with lengths on the order of 100 nucleotides. Complete isolation of the FLP from the remaining side products formed from incomplete coupling (truncation products) and deprotection is not currently achievable for RNA molecules with lengths on the order of 100 nucleotides by standard purification methods (such as chromatography). There is a need for more efficient methods for synthesizing gRNAs.

### SUMMARY

Disclosed herein include methods, compositions, and kits for synthesizing guide RNAs (gRNAs). The method can comprise, for example: providing a first RNA fragment comprising a terminal region comprising a 5′ phosphate moiety, and a second RNA fragment comprising a terminal region comprising a 3′ hydroxyl group, wherein the first RNA fragment, the second RNA fragment, or both, comprises at least a portion of a sequence capable of binding to an RNA-guided endonuclease; providing a splint DNA oligonucleotide comprising a first portion complementary to the first RNA fragment at the terminal region comprising a 5′ phosphate moiety and a second portion complementary to the second RNA fragment at the terminal region comprising a 3′ hydroxyl group; hybridizing the first RNA fragment, the second RNA fragment, and the splint DNA oligonucleotide together to form a first complex; contacting the first and second RNA fragments with a ligase to form a gRNA at a ligation site present between the first and second RNA fragments in the first complex to form a second complex, and digesting the splint DNA oligonucleotide with a DNase to obtain the purified gRNA. In some embodiments, digesting the splint DNA oligonucleotide with the DNase to obtain the gRNA comprises contacting the second complex with the DNase to digest the splint DNA oligonucleotide. In some embodiments, digesting the splint DNA oligonucleotide with the DNase to obtain the gRNA comprises: dissociating the gRNA and the splint DNA oligonucleotide by various means, including but not limited to increased temperature; and digesting the dissociated splint DNA oligonucleotide with the DNase to obtain purified gRNA. The DNase can be a DNase I or a DNase II. The DNase can be, for example, a bacterial DNase. In some embodiments, separating the digested splint DNA oligonucleotide products from the gRNA. In some embodiments, the Tm of the gRNA and the Tm of the splint DNA differ by at least, or by at least about, 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., or 9° C. It can be advantageous for the Tm of the gRNA and the Tm of the splint DNA to differ and thus allows dissociation of the gRNA and the splint DNA for purification of the gRNA. The dissociating of the gRNA and the splint DNA can be carried out at a temperature of or temperature of about, 50° C., 52° C., 54° C., 56° C., 58° C., 60° C., 61° C., 62° C., 63° C., 64° C., 65° C., 66° C., 67° C., 68° C., a number or a range between any two of these values, or more. The Tm of the gRNA and/or the splint DNA can be, for example, 50° C., 52° C., 54° C., 55° C., 56° C., 57° C., 58° C., 59° C., 60° C., 61° C., 62° C., 63° C., 64° C., 65° C., 66° C., 67° C., 68° C., a number or a range between any two of these values.

Described herein includes a method of synthesizing a guide RNA (gRNA), comprising: hybridizing a first RNA fragment, a second RNA fragment and a splint DNA oligonucleotide to form a complex, wherein the first RNA fragment comprises a terminal region comprising a 5′ phosphate moiety, the second RNA fragment comprises a terminal region comprising a 3′ hydroxyl group, wherein the first RNA fragment, the second RNA fragment, or both, comprises at least a portion of a sequence capable of binding to an RNA-guided endonuclease; the splint DNA oligonucleotide comprises (1) a first portion complementary to the first RNA fragment at the terminal region comprising a 5′ phosphate moiety and (2) a second portion complementary to the second RNA fragment at the terminal region comprising a 3′ hydroxyl group, wherein the splint DNA oligonucleotide is no more than 32 nucleotides in length; and ligating the first and second RNA fragments with a ligase at a ligation site present between the first and second RNA fragments in the complex, thereby synthesizing a gRNA. In some embodiments, the hybridizing step and the ligating step occur simultaneously. In some embodiments, the hybridizing step and ligating step occur at different temperature. In some embodiments, the hybridizing step occurs in the presence of the ligase. In some embodiments, the splint DNA oligonucleotide is no more than 26 nucleotides in length. Ligating the first and second RNA fragments can be carried out at various temperatures, for example about 15° C. to about 33° C., and optionally the ligating is carried out at about 15° C., 20° C., 22° C., 24° C., 27° C., 30° C., or 33° C. In some embodiments, the complex has a melting temperature (Tm) greater than 15° C. and lower than 60° C., and optionally the Tm of the complex is lower than 55° C., 50° C., 45° C., 40° C. or 35° C. In some embodiments, the ligating the first and second RNA fragments is carried out for about 6 hours. In some embodiments, the method comprises isolating and/or purifying the gRNA. The purified gRNA can be, for example, at least 80%, 85%, 90%, 95%, or 98% in purity.

In some embodiments, the method comprises separating the gRNA and the splint DNA oligonucleotide after ligating the first and second RNA fragments. In some embodiments, the method further comprises isolating and/or purifying the splint DNA oligonucleotide. In some embodiments, the isolating and/or purifying comprises using a chromatographic method, a size-based separation method, a charge-based separation method, an affinity-based separate method, or a combination thereof. In some embodiments, the method does not comprise using any DNase. In some embodiments, the method does not comprise digesting the splint DNA oligonucleotide after the ligating step. In some embodiments, the method does not comprise digesting the splint DNA oligonucleotide in the complex after the ligating step. In some embodiments, the method does not comprise separating the gRNA and the splint DNA oligonucleotide enzymatically. In some embodiments, the hybridizing a first RNA fragment, a second RNA fragment and a splint DNA oligonucleotide is carried out in the presence of one or more RNase inhibitors. In some embodiments, the first RNA fragment, the second RNA fragment, or both, is about 10 to 90 nucleotides in length.

The first RNA fragment, the second RNA fragment, or both, can be about 10 to 90 nucleotides in length. The ratio between the length of the first RNA fragment and the length of the second RNA fragment can be at least 20:80. In some embodiments, the first RNA fragment is about 20 to 50 nucleotides in length, and the second RNA fragment is about 80 to 50 nucleotides in length. The 5′ phosphate moiety can be 5′-phosphate or 5′-phosphorothioate. Non-limiting examples of the ligase include T4 DNA ligase, T4 RNA ligase I, and T4 RNA ligase II. In some embodiments, the splint DNA oligonucleotide is 20 to 100 nucleotides in length. In some embodiments, the splint DNA oligonucleotide is attached to a solid support.

The ligation site can correspond to a site in a tetraloop portion of a stem-loop structure in the gRNA. In some embodiments, the ligation site corresponds to a site in a helix portion of a stem-loop structure in the gRNA. In some embodiments, the first RNA fragment, the second RNA fragment, or both, comprises at least one secondary structure, and wherein hybridizing the first RNA fragment, the second RNA fragment, and the splint DNA oligonucleotide results in a lower free energy than that of the secondary structure with the lowest free energy. The method can comprise ligating three or more RNA fragments.

In some embodiments, providing the first and second RNA fragments comprises synthesizing the first and/or second RNA fragments through enzymatic synthesis or phosphoramidite chemistry. In some embodiments, providing the first and/or second RNA fragments comprises synthesizing the first and second RNA fragments in a 5′ to 3′ or 3′ to 5′ direction. In some embodiments, providing the first and/or second RNA fragments comprises purifying the first and second fragments after synthesis. In some embodiments, providing the splint DNA oligonucleotide comprises synthesizing the splint DNA oligonucleotide through enzymatic synthesis or phosphoramidite chemistry. In some embodiments, providing the splint DNA oligonucleotide comprises purifying the splint DNA oligonucleotide after synthesis. Purifying can comprise purifying with a chromatographic method, including but not limited to, reversed-phase HPLC, ion-exchange chromatography, size exclusion chromatography, hydrophobic interaction chromatography, affinity chromatography, polyacrylamide gel purification, or any combination thereof.

In some embodiments, the first RNA fragment, the second RNA fragment, or both, comprises one or more modifications in the RNA backbone, including but not limited to, 2′ methoxy (2′ OMe), 2′ fluorine (2′fluoro), 2′-O-methoxy-ethyl (MOE), locked nucleic acids (LNA), unlocked nucleic acids (UNA), bridged nucleic acids, 2′ deoxynucleic acids (DNA), and peptide nucleic acids (PNA). In some embodiments, the first RNA fragment, the second RNA fragment, or both, comprises one or more base modifications, including but not limited to, 2-aminopurine, hypoxanthine, thymine, 2,6-diaminopurine, 2-pyrimidone, and 5-methyl cytosine. In some embodiments, the first RNA fragment, the second RNA fragment, or both, comprises one or more phosphorothioate linkages.

Hybridizing can comprise hybridizing in a solution. In some embodiments, two or more of the splint DNA oligonucleotide, the first RNA fragment, and the second RNA fragments are present in the solution in about an equal concentration.

In some embodiments, ligating the first and second RNA fragments is carried out at about 15° C. to about 45° C., for example at about 37° C. In some embodiments, ligating the first and second RNA fragments is carried out for about 0.1 to about 48 hours. In some embodiments, ligating the first and second RNA fragments is carried out in the presence of a protease, a chelating agent, a crowding agent, or any combination thereof. The chelating agent can comprise EDTA, EGTA, or both. The crowding agent can comprise polyethylene glycol (PEG), Ficoll®, ethylene glycol, dextran, or any combination thereof.

In some embodiments, ligating the first and second RNA fragments proceeds to at least 10% completion. In some embodiments, ligating the first and second RNA fragments proceeds to at least 90% completion.

The method of synthesizing a gRNA can comprise, in some embodiments: providing: (a) a first RNA fragment comprising a terminal region comprising a 3′ hydroxyl group; (b) a second RNA fragment comprising a first terminal region comprising a 5′ phosphate moiety and a second terminal region comprising a 3′ hydroxyl group; (c) a third RNA fragment comprising a terminal region comprising a 5′ phosphate moiety; (d) a first splint DNA oligonucleotide comprising (i) a first portion complementary to the terminal region comprising the 3′ hydroxyl group of the first RNA fragment; and (ii) a second portion complementary to the first terminal region comprising the 5′ phosphate moiety of the second RNA fragment; (e) a second splint DNA oligonucleotide comprising (i) a first portion complementary to the second terminal region comprising the 3′ hydroxyl group of the second RNA fragment; and (ii) a second portion complementary to the terminal region comprising the 5′ phosphate moiety of the third RNA fragment; and (f) a ligase; hybridizing the first, second, and third RNA fragments and the first and second splint DNA oligonucleotides to form a first complex having a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment; ligating the first and second RNA fragments, and the second and third RNA fragments, respectively, to form a gRNA using the ligase at the first and second ligation sites in the first complex to form a second complex; and digesting the first and second splint DNA oligonucleotides with a DNase to obtain the gRNA. In some embodiments, the hybridizing step and the ligating step occur simultaneously. In some embodiments, the hybridizing step and ligating step occur at different temperature. In some embodiments, the hybridizing step occurs in the presence of the ligase.

Digesting the first and second splint DNA oligonucleotides with the DNase to obtain the gRNA can comprise contacting the second complex with the DNase to digest the first and second splint DNA oligonucleotides. In some embodiments, digesting the first and second splint DNA oligonucleotides with the DNase to obtain the gRNA comprises: dissociating the gRNA and the first and/or second splint DNA oligonucleotides by various means, including but not limited to increased temperature; and digesting the dissociated first and/or second splint DNA oligonucleotides with the DNase to obtain purified gRNA.

In some embodiments, the method comprises separating the digested splint DNA oligonucleotide products from the gRNA. The DNase can be DNase I or DNase II. The DNase can be a bacterial DNase.

Disclosed herein includes a method of synthesizing a guide RNA (gRNA), the method comprising: hybridizing a first RNA fragment, a second RNA fragment, a third RNA fragment, a first splint DNA oligonucleotide and a second first splint DNA oligonucleotide to form a complex, wherein (a) the first RNA fragment comprises a terminal region comprising a 3′ hydroxyl group, (b) the second RNA fragment comprises a first terminal region comprising a 5′ phosphate moiety and a second terminal region comprising a 3′ hydroxyl group, (c) a third RNA fragment comprises a terminal region comprising a 5′ phosphate moiety, (d) the first splint DNA oligonucleotide comprises (i) a first portion complementary to the terminal region comprising the 3′ hydroxyl group of the first RNA fragment; and (ii) a second portion complementary to the first terminal region comprising the 5′ phosphate moiety of the second RNA fragment; (e) the second splint DNA oligonucleotide comprises (i) a first portion complementary to the second terminal region comprising the 3′ hydroxyl group of the second RNA fragment; and (ii) a second portion complementary to the terminal region comprising the 5′ phosphate moiety of the third RNA fragment, wherein the complex comprises (i) a first ligation site present between the 3′ hydroxyl group of the first RNA fragment and the 5′ phosphate group of the second RNA fragment, and (i) a second ligation site present between the 3′ hydroxyl group of the second RNA fragment and the 5′ phosphate group of the third RNA fragment, and wherein each of the first splint DNA oligonucleotide and the second splint DNA oligonucleotide is no more than 32 nucleotides in length; and ligating the first and second RNA fragments, and the second and third RNA fragments, respectively, with a ligase at the first and second ligation sites in the complex, thereby synthesizing a gRNA. In some embodiments, the hybridizing step and the ligating step occur simultaneously. In some embodiments, the hybridizing step and ligating step occur at different temperature. In some embodiments, the hybridizing step occurs in the presence of the ligase. In some embodiments, the first splint DNA oligonucleotide, the second splint DNA oligonucleotide, or both, is no more than 26 nucleotides in length. In some embodiments, the ligating is carried out at about 15° C. to about 33° C., and optionally the ligating is carried out at about 15° C., 20° C., 22° C., 24° C., 27° C., 30° C., or 33° C. In some embodiments, the complex has a melting temperature (Tm) greater than 15° C. and lower than 60° C., and optionally the Tm of the complex is lower than 55° C., 50° C., 45° C., 40° C. or 35° C. In some embodiments, the ligating is carried out for about 6 hours. In some embodiments, the method comprises isolating and/or purifying the gRNA. The purified gRNA can be at least 80%, 85%, 90%, 95%, or 98% in purity. In some embodiments, the method comprises after ligating, separating the gRNA and one or more of the first and second splint DNA oligonucleotides. The method, in some embodiments, further comprises isolating and/or purifying one or more of the first and second splint DNA oligonucleotides. In some embodiments, the isolating and/or purifying comprises using a chromatographic method, a size-based separation method, a charge-based separation method, an affinity-based separation method, or a combination thereof. In some embodiments, the method does not comprise using any DNase. In some embodiments, the method does not comprise digesting one or more of the first and second splint DNA oligonucleotides after the ligating step. In some embodiments, the method does not comprise digesting one or more of the first and second splint DNA oligonucleotides in the complex after the ligating step. In some embodiments, the method does not comprise separating the gRNA and one or more of the first and second splint DNA oligonucleotides enzymatically. In some embodiments, the hybridizing step is carried out in the presence of one or more RNase inhibitors.

In some embodiments, the gRNA comprises 5′ to 3′ the first RNA fragment linked to the second RNA fragment by a first phosphodiester bond, and the second RNA fragment linked to the third RNA fragment by a second phosphodiester bond. In some embodiments, the first phosphodiester bond is formed between the 3′ hydroxyl group of the first RNA fragment and the 5′ phosphate group of the second RNA fragment, and wherein the second phosphodiester bond is formed between the 3′ hydroxyl group of the second RNA fragment and the 5′ phosphate group of the third RNA fragment. In some embodiments, the first ligation site corresponds to a site in a first stem-loop structure, wherein the first stem-loop structure is formed by hybridization of a minimum CRISPR repeat sequence and a minimum tracrRNA sequence in the gRNA. In some embodiments, the site in the first stem-loop structure is in a tetraloop portion or in a helix portion. In some embodiments, the second ligation site corresponds to a site in a second stem-loop structure, wherein the second stem-loop structure is present in a tracrRNA sequence of the gRNA. In some embodiments, the site in the second stem-loop structure is in a tetraloop portion or in a helix portion.

In some embodiments, one or more of the first RNA fragment, the second RNA fragment, and the third RNA fragment comprises at least one secondary structure, and wherein the complex formed by hybridizing the first, second, and third RNA fragments and the first and second splint oligonucleotides has a lower free energy than that of the secondary structure with the lowest free energy. The gRNA can be a single gRNA (sgRNA).

Also provided herein include a method of synthesizing an sgRNA for use with an RNA-guided endonuclease, comprising: providing a first complex comprising a first RNA fragment, a second RNA fragment, a third RNA fragment, a first splint oligonucleotide, and a second splint oligonucleotide, wherein (a) the first RNA fragment comprises (i) a terminal region comprising a 3′ hydroxyl group; (b) the second RNA fragment comprises (i) a first terminal region comprising a 5′ phosphate moiety, and (ii) a second terminal region comprising a 3′ hydroxyl group; (c) the third RNA fragment comprises (i) a terminal region comprising a 5′ phosphate moiety; (d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and (e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment, wherein the first complex is formed by hybridization of (a)(i) and (d)(i), (b)(i) and (d)(ii), (b)(ii) and (e)(i), and (c)(i) and (e)(ii), wherein the first complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment; ligating the first RNA fragment and the second RNA fragment at the first ligation site and ligating the second RNA fragment and the third RNA fragment at the second ligation site to form a second complex comprising a sgRNA comprising from 5′ to 3′: a spacer sequence and an invariable sequence that binds an RNA-guided endonuclease; the invariable sequence comprising a stem loop formed between a crRNA repeat sequence and a tracrRNA anti-repeat sequence, and a 3′ tracrRNA sequence comprising at least one stem-loop; and digesting the first and second splint DNA oligonucleotides with a DNase to obtain the sgRNA.

In some embodiments, digesting the first and second splint DNA oligonucleotides with the DNase to obtain the sgRNA comprises contacting the second complex with the DNase to digest the first and second splint DNA oligonucleotides. In some embodiments, digesting the first and second splint DNA oligonucleotides with the DNase to obtain the sgRNA comprises: dissociating the sgRNA and the first and/or second splint DNA oligonucleotides by various means, including but not limited to increased temperature; and digesting the dissociated first and/or second splint DNA oligonucleotides with the DNase to obtain purified sgRNA. The method, in some embodiments, comprises separating the digested splint DNA oligonucleotide products from the sgRNA. The DNase can be a DNase I or a DNase II. The DNase can be a bacterial DNase.

Disclosed herein include a method of synthesizing a single guide RNA (sgRNA) for use with an RNA-guided endonuclease, the method can comprising: providing a complex comprising a first RNA fragment, a second RNA fragment, a third RNA fragment, a first splint oligonucleotide, and a second splint oligonucleotide, wherein (a) the first RNA fragment comprises (i) a terminal region comprising a 3′ hydroxyl group; (b) the second RNA fragment comprises (i) a first terminal region comprising a 5′ phosphate moiety, and (ii) a second terminal region comprising a 3′ hydroxyl group; (c) the third RNA fragment comprises (i) a terminal region comprising a 5′ phosphate moiety; (d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and (e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment, wherein each of the first splint oligonucleotide and the second splint DNA oligonucleotide is no more than 32 nucleotides in length, wherein the complex is formed by hybridization of (a)(i) and (d)(i), (b)(i) and (d)(ii), (b)(ii) and (e)(i), and (c)(i) and (e)(ii), wherein the complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment; and ligating the first RNA fragment and the second RNA fragment at the first ligation site and ligating the second RNA fragment and the third RNA fragment at the second ligation site to synthesize a sgRNA comprising from 5′ to 3′: a spacer sequence and an invariable sequence that binds an RNA-guided endonuclease; the invariable sequence comprising a stem loop formed between a crRNA repeat sequence and a tracrRNA anti-repeat sequence, and a 3′ tracrRNA sequence comprising at least one stem-loop. In some embodiments, the first splint DNA oligonucleotide, the second splint DNA oligonucleotide, or both, is no more than 26 nucleotides in length. In some embodiments, the ligating is carried out at about 15° C. to about 33° C., and optionally the ligating is carried out at about 15° C., 20° C., 22° C., 24° C., 27° C., 30° C., or 33° C. In some embodiments, the complex has a melting temperature (Tm) greater than 15° C. and lower than 60° C., and optionally the Tm of the complex is lower than 55° C., 50° C., 45° C., 40° C. or 35° C. In some embodiments, the ligating is carried out for about 6 hours. In some embodiments, the method comprises isolating and/or purifying the sgRNA. The purified sgRNA can be at least 80%, 85%, 90%, 95%, or 98% in purity. In some embodiments, the method comprises after ligating, separating the sgRNA and one or more of the first and second splint oligonucleotides. In some embodiments, the method further comprises isolating and/or purifying one or more of the first and second splint oligonucleotides. In some embodiments, the isolating and/or purifying comprises using a chromatographic method, a size-based separate method, a charge-based separation method, an affinity-based separation method, or a combination thereof. In some embodiments, the method does not comprise using any DNase. In some embodiments, the method does not comprise digesting one or more of the first and second splint oligonucleotides after the ligating step. In some embodiments, the method does not comprise digesting one or more of the first and second splint oligonucleotides in the complex after the ligating step. In some embodiments, the method does not comprise separating the sgRNA and one or more of the first and second splint oligonucleotides enzymatically. In some embodiments, the hybridization of (a)(i) and (d)(i), (b)(i) and (d)(ii), (b)(ii) and (e)(i), and (c)(i) and (e)(ii) is carried out in the presence of one or more RNase inhibitors.

The Tm of the gRNA and the Tm of the first and/or second splint DNA oligonucleotide can differ by at least, or by at least about, 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., or 9° C. It can be advantageous for the Tm of the gRNA and the Tm of the first and second splint DNA oligonucleotides to differ and thus allows dissociation of the gRNA and the splint DNA oligonucleotides for purification of the gRNA. The dissociating of the sgRNA and the first and/or second splint DNA can be carried out at a temperature of or temperature of about, 50° C., 52° C., 54° C., 56° C., 58° C., 60° C., 61° C., 62° C., 63° C., 64° C., 65° C., 66° C., 67° C., 68° C., a number or a range between any two of these values, or more. The Tm of one or more of the sgRNA, the first, and the second splint DNA can be, for example, 50° C., 52° C., 54° C., 55° C., 56° C., 57° C., 58° C., 59° C., 60° C., 61° C., 62° C., 63° C., 64° C., 65° C., 66° C., 67° C., 68° C., a number or a range between any two of these values.

In some embodiments, the first ligation site corresponds to a site in the stem loop formed between the crRNA repeat sequence and the tracrRNA anti-repeat sequence. In some embodiments, the first ligation site corresponds to a site in the 5′ stem of the stem loop, in the tetraloop of the stem loop, or in the 3′ stem of the stem loop. The 3′ tracrRNA sequence can comprise a first stem loop, a second stem loop, and a third stem loop.

In some embodiments, the second ligation site corresponds to a site in the first stem loop, the second stem loop, or the third stem loop. In some embodiments, the second ligation site corresponds to a site in the second stem loop, wherein the site is in the 5′ stem of the second stem loop, a site in the tetraloop of the second stem loop, or a site in the 3′ stem of the second stem loop. In some embodiments, the second ligation site corresponds to a site adjacent to the 5′ base of the second stem loop or adjacent to the 3′ base of the second stem loop. In some embodiments, the first RNA fragment comprises a nucleotide sequence that is 5′ the first ligation site. In some embodiments, the second RNA fragment comprises a nucleotide sequence that is between the first ligation site and the second ligation site. In some embodiments, the third RNA fragment comprises a nucleotide sequence that is 3′ to the second ligation site. The terminal region of (a)(i) can comprise a nucleotide sequence of about 10 to about 30 nucleotides located at the 3′ end of the first RNA fragment. In some embodiments, the terminal region of (a)(i) comprises the spacer sequence of the sgRNA. In some embodiments, the terminal region of (a)(i) does not comprise the spacer sequence of the sgRNA. In some embodiments, the first portion of (d)(i) is perfectly complementary to the terminal region of (a)(i), or has 1, 2, or 3 mismatches relative to the terminal region of (a)(i). In some embodiments, the first terminal region of (b)(i) comprises a nucleotide sequence of about 10 to about 30 nucleotides located at the 5′end of the second RNA fragment. In some embodiments, the second portion of (d)(ii) is perfectly complementary to the first terminal region of (b)(i), or has 1, 2, or 3 mismatches relative to the terminal region of (d)(ii). In some embodiments, the second terminal region of (b)(ii) comprises a nucleotide sequence of about 10 to about 30 nucleotides located at the 3′ end of the second RNA fragment. In some embodiments, the first portion of (e)(i) is perfectly complementary to the second terminal region of (b)(ii), or has 1, 2, or 3 mismatches relative to the terminal region of (b)(ii). In some embodiments, the terminal region of (c)(i) comprises a nucleotide sequence of about 10 to about 40 nucleotides located at the 5′end of the third RNA fragment. In some embodiments, the second portion of (e)(ii) is perfectly complementary to the terminal region of (c)(i), or has 1, 2, or 3 mismatches relative to the terminal region of (c)(i). The first RNA fragment, the second RNA fragment, and the third RNA fragment are each independently about 10 to about 90 nucleotides. The ligase can be, for example, a T4 DNA ligase, T4 RNA ligase I, or T4 RNA ligase II.

In some embodiments, the first splint oligonucleotide and the second splint oligonucleotide are each independently about 20 to about 100 nucleotides. In some embodiments, the gRNA is about 30 to about 160 nucleotides in length. In some embodiments, the gRNA comprises a sequence that is complementary to a sequence in a target DNA. The target DNA can be mammalian DNA or human DNA.

The RNA-guided endonuclease can be a small Cas nuclease or a small RNA-guided endonuclease. The RNA-guided endonuclease can be, for example, a Cas9, a Cas12, aCas13, and variants thereof. In some embodiments, the RNA-guided endonuclease is a Streptococcus pyogenes Cas9 (SpyCas9) or a Staphylococcus aureus (SaCas9). In some embodiments, the RNA-guided endonuclease is a variant of Cas9, and the variant of Cas9 is selected from the group consisting of a small Cas9, a dead Cas9 (dCas9), and a Cas9 nickase. In some embodiments, the invariable sequence comprises the nucleotide sequence of SEQ ID NO: 1, or a nucleotide sequence having up to 1, 2, 3, 4, 5, 6, 7, 8, 9, or 10 nucleotide deletions, insertions, or substitutions relative to the nucleotide sequence of SEQ ID NO: 1.

Providing the first RNA fragment, the second RNA fragment, and the third RNA fragment can comprise synthesis of the RNA fragments using enzymatic synthesis or phosphoramidite chemistry, optionally comprising purifying the RNA fragments after synthesis. In some embodiments, synthesis of the RNA fragments using phosphoramidite chemistry comprises: (i) synthesis of the first RNA fragment, synthesis of the second RNA fragment, and synthesis of the third RNA fragment in a 5′ to 3′ or in a 3′ to 5′ direction; or (ii) synthesis of the first RNA fragment in a 5′ to 3′ or in a 3′ to 5′ direction and synthesis of the second RNA fragment and synthesis of the third RNA fragment in a 3′ to 5′ direction.

Providing the first and second splint oligonucleotides can comprise synthesis of the oligonucleotides using enzymatic synthesis or phosphoramidite chemistry, optionally comprising purifying the oligonucleotides after synthesis. In some embodiments, the first RNA fragment, the second RNA fragment, and/or the third RNA fragment, comprises one or more modifications in the RNA backbone, including but not limited to 2′ methoxy (2′OMe), 2′ fluorine (2′fluoro), 2′-O-methoxy-ethyl (MOE), Locked Nucleic Acids (LNA), Unlocked Nucleic Acids (UNA), bridged nucleic acids, 2′deoxynucleic acids (DNA), and peptide nucleic acids (PNA). In some embodiments, the first RNA fragment, the second RNA fragment, and/or the third RNA fragment, comprises one or more base modifications, including but not limited to, 2-aminopurine, hypoxanthine, thymine, 2,6-diaminopurine, 2-pyrimidone, and 5-methyl cytosine. In some embodiments, the first RNA fragment, the second RNA fragment, and/or the third RNA fragment, comprises at least one phosphorothioate linkage.

In some embodiments, hybridizing is performed in a solution, and wherein the hybridizing is performed with or without an annealing step. In some embodiments, the annealing step comprises (i) heating the solution to about 80° C. to about 95° C. for a period of time less than about 10 minutes; and/or (ii) cooling the solution at a rate of about 0.1° C. to about 2° C. per second to a temperature used for the ligation.

In some embodiments, two or more of the first splint oligonucleotide, the second splint oligonucleotide, the first RNA fragment, the second RNA fragment, and the third RNA fragment are present in the solution in an about equal concentration. The ligation can be carried out at about 15° C. to about 45° C., and/or for about 0.1 to about 48 hours. The ligation can further comprise using one or more of a protease, a chelating agent, and a crowding agent. The chelating agent can comprise EDTA, EGTA, or both. The crowding agent can comprise polyethylene glycol (PEG), Ficoll®, ethylene glycol, dextran, or any combination thereof. In some embodiments, the ligation proceeds to at least about 10% completion.

The method can further comprises purifying the gRNA or the sgRNA after synthesis. In some embodiments, purifying the gRNA or sgRNA comprises purifying using a chromatographic method, including but not limited to, reversed-phase HPLC, ion-exchange chromatography, size exclusion chromatography, hydrophobic interaction chromatography, affinity chromatography, or polyacrylamide gel purification, or any combination thereof.

### BRIEF DESCRIPTION OF THE DRAWINGS

FIG. 1 is a non-limiting schematic illustration showing the synthesis of a gRNA molecule by ligating two RNA fragments using a splint oligonucleotide.

FIG. 2 is a non-limiting schematic illustration showing the synthesis of a gRNA molecule by ligating three RNA fragments using two splint oligonucleotides.

FIG. 3 is a non-limiting schematic illustration showing the generation of an invariable RNA construct and subsequent ligation with an additional RNA fragment, using a splint oligonucleotide, to generate a gRNA molecule.

FIG. 4A-FIG. 4B are non-limiting schematic illustrations for the splint-mediated ligation between modified RNA fragments. In FIG. 4A, three modified RNA fragments (RNA 1, RNA 2, and RNA 3 fragments) and two DNA splint oligonucleotides (DNA splint 1 and DNA splint 2) are used to generate a modified single gRNA (sgRNA). In FIG. 4B, two modified RNA fragments (RNA 1 and RNA 2 fragments) and one DNA splint oligonucleotides (DNA splint 1) are used to generate a modified single gRNA (sgRNA).

FIG. 5A shows the sequence of an exemplary gRNA and the sequences of three RNA fragments (RNA1 fragment (SEQ ID NO: 62), RNA2 fragment (SEQ ID NO: 63) and RNA3 fragment (SEQ ID NO: 64)) that can be used in a ligation and DNase digestion method disclosed herein to generate such an gRNA. The ligation site between RNA1 and RNA2, and the ligation site between RNA2 and RNA3 are shown in FIG. 5A. FIG. 5B is a non-limiting schematic illustration for the splint-mediated ligation among three RNA fragments (RNA1, RNA2, and RNA3 fragments) and two DNA splint oligonucleotides (DNA splint 1 and DNA splint 2) using T4 RNA ligase 2. The resulting long RNA is synthesized by hybridization of RNA1, RNA2 and RNA3 with DNA splint 1 and DNA splint 2, and subsequent ligation at a ligation site between the 3′ terminus of RNA 1 and the 5′ terminus of RNA2, and a ligation site between the 3′ terminus of RNA2 and the 5′ terminus of RNA3.

FIG. 6A-FIG. 6B show ligation purification by IE HPLC: panel 1 shows pre-purification ligation components (RNA1 fragment, RNA2 fragment, RNA3 fragment, DNA Splint 1, and DNA Splint 2), panel 2 shows components of crude ligation reaction (DNA Splint 1, DNA Splint 2, and ligated gRNA reaction product), panel 3 shows HPLC separation of DNA Splints and Intermediates & Ligated Oligo, and panel 4 shows post-purified analysis of the components shown in the blue box of panel 3 (intermediates & ligated oligo).

FIG. 7A-FIG. 7B show ligation purification with high-resolution column.

FIG. 8 shows that DNase treatment resulted in improvement in HPLC purification for the ligation reaction.

FIG. 9 shows LC/MS data of purified gRNA (about 1 mg).

FIG. 10 shows scaling up ligation reaction for purified gRNA (about 3 mg).

FIG. 11A-FIG. 11B show non-limiting examples of two-RNA fragment designs for synthesizing a gRNA of interest. FIGS. 11A and 11B provide the sequence of a gRNA of interest and the sequences of two RNA fragments (RNA1 and RNA2 fragments) that can be used in a ligation and DNase digestion method disclosed herein to generate the gRNA of interest. In FIG. 11A, one RNA fragment is 40 nucleotides in length (SEQ ID NO: 65) and the other RNA fragment is 60 nucleotides in length (SEQ ID NO: 66), and the ligation site between the two RNA fragments is shown. In FIG. 11B, one RNA fragment is 35 nucleotides in length (SEQ ID NO: 67) and the other RNA fragment is 65 nucleotides in length (SEQ ID NO: 68), and the ligation site between the two RNA fragments is shown.

FIG. 12A-FIG. 12B show non-limiting examples of two-RNA fragment design for synthesizing a gRNA of interest. FIG. 12A shows a 35-65 design in which the first RNA fragment (RNA1) is 35 nucleotides in length (SEQ ID NO: 69) and the second fragment (RNA2) is 65 nucleotides in length (SEQ ID NO: 70). FIG. 12B shows a 40-60 design in which RNA1 is 40 nucleotides in length (SEQ ID NO: 71) and RNA2 is 60 nucleotides in length (SEQ ID NO: 72).

FIG. 13A-FIG. 13B show purification by Ion Exchange HPLC (IE-HPLC) of crude of ligation reaction. FIG. 13A shows HPLC profile with DNase treatment. FIG. 13B shows HPLC profile after spin column purification.

FIGS. 14A-C show purification by IE HPLC of crude of ligation reaction carried out at 33° C., 30° C. and 27° C., respectively.

FIGS. 15A-B show purification by IE HPLC of crude of ligation reaction carried out with short DNA splints (a 33-mer and a 27-mer) at 27° C. and 30° C., respectively.

FIGS. 15C-D show the purification by IE HPLC of crude of ligation reaction carried out with very short DNA splints (a 26-mer and a 23-mer) at 27° C. and 30° C., respectively.

FIGS. 16A-B show LC/MC data of purified reaction products from an exemplary very short splint-mediated ligation reaction. FIG. 16A shows LC/MC data of the large-scale reaction. FIG. 16B shows deconvoluted MS analysis of full-length product.

FIG. 17A shows preparative IE-HPLC chromatogram of the ligation reaction mixture (no DNase enzyme was added). FIG. 17B shows HPLC separation of full length sgRNA with no DNA splints contamination observed. FIG. 17 C shows HPLC separation of DNA splints with no sgRNA observed.

FIG. 18A shows LC/MC analysis of pure mT2-sgRNA and FIG. 18B shows the MS analysis of single peak.

FIG. 19A shows HPLC of crude hT5 reaction. FIG. 19B shows HPLC purification of full length hT5 gRNA at 20 ml scale.

FIGS. 20A-B show LC/MC analysis of pure hT5-sgRNA.

FIG. 21A shows HPLC of crude IL2RG reaction. FIG. 21B shows HPLC purification of full length IL2RG gRNA at 20 ml scale.

FIGS. 22A-B show LC/MC analysis of pure hT5-sgRNA.

FIGS. 23A-D show purification by IE HPLC of crude of ligation reaction carried out at 24° C., 22° C., 20° C., and 15° C., respectively.

FIGS. 24A-F show the UPLC trace and HPLC/MS chromatograms of a gRNA synthetized using a 35-65 two-RNA fragment design (FIGS. 24A-B), a 40-60 two-RNA fragment design (FIGS. 24C-D), and the three-RNA fragment design (FIGS. 24E-F).

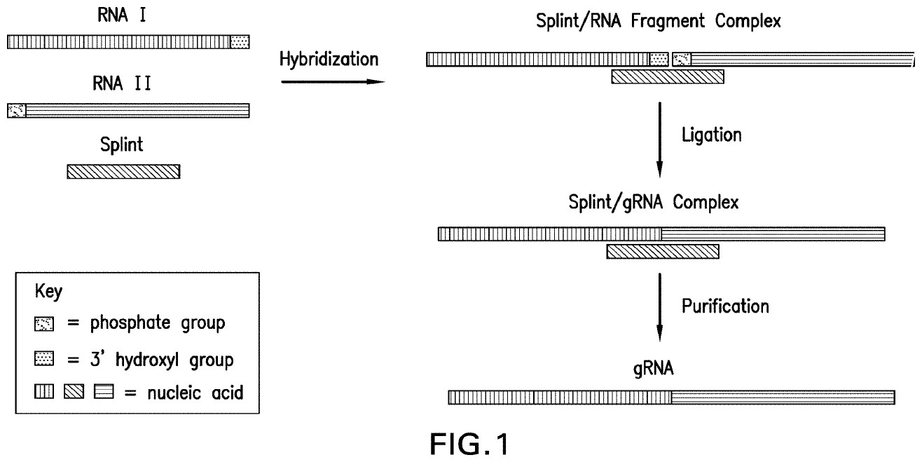

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods, compositions and kits for generating RNAs, for example moderate length RNAs (mlRNAs), by ligating RNA fragments using one or more splint DNA oligonucleotides and one or more ligases. The mlRNAs can be, for example, guide RNAs (gRNAs), including single gRNAs (sgRNAs). In some embodiments, one or more of the RNA fragments comprise at least a portion of a sequence that can bind to an RNA-guided endonuclease. In some embodiments, one or more of the RNA fragments comprise a spacer sequence for targeting a target sequence in a target DNA (e.g., genomic DNA molecule).

Current approaches for the synthesis of mlRNAs include intracellular transcription of an exogenous plasmid or solid-phase synthesis using phosphoramidite chemistry. Chemical synthesis of mlRNAs is not desirable. For example, if the phosphoramidite chemistry being used has a coupling efficiency of ˜0.99^{X} (where X is the number of nucleotides), the overall synthesis process would be expected to yield approximately 30-40% full-length product (FLP) when synthesizing RNAs with lengths on the order of 100 nucleotides. Complete isolation of the FLP from the remaining side products formed from incomplete coupling (truncation products) and deprotection is not currently achievable for RNA molecules with lengths on the order of 100 nucleotides using standard purification methods (e.g. chromatography). Methods, compositions and kits disclosed herein are efficient in synthesizing mlRNAs, improves the yield of full-length products, and decreases the number of truncation products as compared to the existing technologies. The methods disclosed herein can be used in synthesis of both unmodified mlRNAs (e.g., a gRNA or sgRNA) and mlRNAs comprising one or more chemical modifications, such as, a backbone modification (e.g., a phosphorothioate linkage) and/or a nucleoside modification (e.g., 2′-O-methylation).

As disclosed herein, sgRNAs for use with an RNA-guided endonuclease (e.g., Cas9) can be effectively synthesized using a splint-mediated ligation approach by ligating two or more RNA fragments using one or more splint DNA oligonucleotides, and one or more ligases. In some embodiments, the ligation comprises two RNA fragments, one splint DNA oligonucleotide, and a ligase, wherein the two RNA fragments hybridize to the splint DNA oligonucleotide and form a first complex comprising a ligation site, the two RNA fragments are ligated at the ligation site to form a second complex. In some embodiments, the splint DNA oligonucleotide is digested after gRNA is synthesized, for example the splint DNA oligonucleotide can be treated with a DNases to obtain the gRNA for use with an RNA-guided endonuclease. In some embodiments, digesting the splint DNA oligonucleotide with the DNase to obtain the gRNA includes contacting the second complex with the DNase to digest the splint DNA oligonucleotide. In some embodiments, the gRNA and the splint DNA oligonucleotide are dissociated first, and the dissociated splint DNA oligonucleotide is digested by the DNase to obtain free gRNA (e.g., gRNA that is not bound by the splint DNA oligonucleotide). In some embodiments (e.g., when the splint DNA oligonucleotide is no more than 32 nucleotides in length), free gRNA can be obtained without the need of a DNase treatment. For example, the method (including the ligation reaction) does not comprise the use of a DNase and/or the method does not comprise adding a DNase into a ligation reaction mixture prior to or after the ligation reaction. The obtained gRNA can, for example, comprise a nucleotide sequence that is 5′ the ligation site and a nucleotide sequence that is 3′ the ligations site, wherein a first RNA fragment corresponds to the nucleotide sequence that is 5′ the ligation site, and second RNA fragment corresponds to the nucleotide sequence that is 3′ the ligation site, wherein ligation at the ligation site enables joining of the first and second RNA fragments to form the nucleotide sequence of the gRNA.

The ligation can comprise, for example, three RNA fragments, two splint DNA oligonucleotides, and a ligase, wherein the three RNA fragments hybridize the two splint oligonucleotides to form a first complex comprising a first and second ligation site, and wherein the ligase results in ligation at the first and second ligation site, thereby forming a second complex comprising the gRNA. In some embodiments, the ligation is followed by digesting the first and/or second splint DNA oligonucleotides with a DNase to obtain the gRNA. In some embodiments, digesting the first and/or the second splint DNA oligonucleotide comprises contacting the second complex with the DNase to digest the first and/or the second splint DNA oligonucleotide. In some embodiments, the first and/or the second splint DNA oligonucleotide is dissociated from the gRNA, and the dissociated first and/or second splint DNA oligonucleotide is digested by the DNase to obtain free gRNA (e.g., gRNA that is not bound by either the first or the second splint DNA oligonucleotides). In some embodiments (e.g., when the splint DNA oligonucleotide is no more than 32 nucleotides in length), free gRNA can be obtained without a DNase treatment. For example, the method (including the ligation reaction) does not comprise the use of a DNase and/or the method does not comprise adding a DNase into a ligation reaction mixture prior to or after the ligation reaction. In some embodiments, the gRNA comprises a nucleotide sequence that is 5′ the first ligation site, a nucleotide sequence that is 3′ the first ligation site and 5′ the second ligation site, and a nucleotide sequence that is 3′ the second ligation site, wherein a first RNA fragment corresponds to the nucleotide sequence that is 5′ the first ligation site, a second RNA fragment corresponds to the nucleotide sequence that is 3′ the first ligation site and 5′ the second ligation site, and a third RNA fragment corresponds to the nucleotide sequence 3′ the second ligation site, wherein ligation at the first and second ligation sites enables joining of the first, second, and third RNA fragments to form the nucleotide sequence of the gRNA.

In some embodiments, the nucleotide sequence of the gRNA comprises 5′ to 3′: a spacer sequence for targeting a target site in a nucleic acid molecule (e.g., genomic DNA molecule) and an invariable sequence that binds the RNA-guided endonuclease, the invariable sequence comprising 5′ to 3′: a stem loop formed between a CRISPR repeat sequence and a tracrRNA anti-repeat sequence, and a tracrRNA comprising at least one stem loop. In some aspects, the splint-mediated ligation approach provides at least one RNA fragment, or a combination of RNA fragments, comprising the spacer sequence; and at least one RNA fragment, or a combination of RNA fragments, comprising an invariable sequence.

The splint-mediated ligation method disclosed herein can comprise placement of a ligation site in a gRNA (e.g., sgRNA), the ligation site within or proximal a stem loop in the invariable sequence of the gRNA (e.g., within or proximal a stem loop formed between the CRISPR repeat sequence and the tracrRNA anti-repeat sequence; e.g., within or proximal a stem loop of the tracrRNA). As described herein, placement of the ligation site in a stem loop prevents formation of secondary structure in the RNA fragments (i.e., the RNA fragments joined at the ligation site) that would prevent or disfavor hybridization of the RNA fragments with a splint oligonucleotide. For example, disruption of a stem loop by a ligation site provides RNA fragments (i.e., RNA fragments joined at the ligation site) that have (i) minimal secondary structure; and/or (ii) secondary structure with free energy higher that is higher than the free energy resulting from hybridization of the RNA fragments and the splint oligonucleotide.

In some embodiments, the splint-mediated ligation method disclosed herein comprises placement of a first and second ligation site in the sgRNA, the first and second ligation site each within or proximal a stem loop in the invariable sequence of the sgRNA (e.g., a stem loop formed between the CRISPR repeat sequence and the tracrRNA anti-repeat sequence; e.g., a stem loop of the tracrRNA), such that placement of the first ligation site disrupts stem loop formation in an RNA fragment comprising a nucleotide sequence 5′ the first ligation site and an RNA fragment comprising a nucleotide sequence 3′ the first ligation site; and placement of the second ligation site disrupts stem loop formation in the RNA fragment comprising a nucleotide sequence 5′ the second ligation site and an RNA fragment comprising a nucleotide sequence 3′ the second ligation site, such that, formation of secondary structure in each or all RNA fragments used in the splint-mediated ligation reaction is disfavored relative to hybridization with a splint oligonucleotide.

### Definition

As used herein, the term “about” means plus or minus 5% of the provided value.

As used herein, the term “moderate length RNA (mlRNA)” refers an RNA molecule with a length of about 30 to about 160 nucleotides. An mlRNA can be or be about 30, 35, 40, 45, 50, 55, 60, 65, 70, 75, 80, 85, 90, 91, 92, 93, 94, 95, 96, 97, 98, 99, 100, 101, 102, 103, 104, 105, 106, 107, 108, 109, 110, 115, 120, 125, 130, or a number or a range between any two of these values, nucleotides in length. For example, an mlRNA can be a RNA (e.g., gRNA) of 100 nucleotides in length.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

As used herein, a “secondary structure” of a nucleic acid molecule (e.g., an RNA fragment, or a gRNA) refers to the base pairing interactions within the nucleic acid molecule.

As used herein, the term “target DNA” refers to a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

As used herein, the term “Cas endonuclease” or “Cas nuclease” refers to an RNA-guided DNA endonuclease associated with the CRISPR adaptive immunity system.

Unless otherwise indicated “nuclease” and “endonuclease” are used interchangeably herein to refer to an enzyme which possesses endonucleolytic catalytic activity for polynucleotide cleavage.

As used herein, the term “cleavage” refers to the breakage of the covalent backbone of a DNA molecule. The cleavage can be a single-stranded cleavage or a double-stranded cleavage. For example, a double-stranded cleavage can occur as a result of two distinct single-stranded cleavage events.

As used herein, the term “domain” refers to a segment of a protein or a nucleic acid. Unless otherwise indicated, a domain need not have any specific functional property.

As used herein, the term “splint oligonucleotide” refers to an oligonucleotide that, when hybridized to other polynucleotides (e.g., RNA fragments), acts as a “splint” to position the ends of the polynucleotides next to one another so that they can be ligated together. The splint oligonucleotide can be any oligomer that can hybridize with the polynucleotides through Watson-Crick base-pairing interactions. The splint oligonucleotide can be DNA, RNA, non-natural, or artificial nucleic acids (e.g., peptide nucleic acids). The splint oligonucleotide can include a nucleotide sequence that is partially complimentary to nucleotide sequences from two or more different oligonucleotides. In general, an RNA ligase, a DNA ligase, or another variety of ligase can be used to ligate two nucleotide sequences together. In some embodiments, the splint oligonucleotide is a DNA oligonucleotide that can be digested by DNase. In some embodiments, the splint oligonucleotide is an oligonucleotide susceptible to digestion by DNase. The splint oligonucleotide can include one or more modifications on one or more sugar moieties and/or one or more bases. In some embodiments, the splint oligonucleotide comprises one or more modified and/or non-natural bases.

As used herein, the “spacer” or “variable region” of a gRNA includes a nucleotide sequence that is complementary to a specific sequence within a target DNA (the complementary strand of the target DNA). In some aspects, the spacer confers target specificity to the gRNA combined with an RNA-guided endonuclease, enabling the RNA-guided endonuclease to cleave at the target site targeted by the spacer in the target DNA. As used herein, the term “spacer” is used interchangeably with the term “spacer sequence.”

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹M, 10⁻¹⁰ M, 10⁻¹¹ M, 10⁻¹² M, 10⁻¹³ M, 10⁻¹⁴ M, 10⁻¹⁵M, or a number or a range between any two of these values. Kd can be dependent on environmental conditions, e.g., pH and temperature. “Affinity” refers to the strength of binding, and increased binding affinity is correlated with a lower Kd.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

It is advantageous to obtain long RNAs (e.g., mlRNAs including gRNAs) in high purity, which is not only important for meeting regulatory requirements but also allows chemical modifications currently only used in short oligonucleotides (e.g., siRNAs and anti-sense RNAs) to be applied in long RNAs. Highly purified long RNAs (e.g., mlRNAs including gRNAs) can also allow the efficient production and use of RNA/DNA hybrids in gene editing (e.g., chRDNA (CRISPR hybrid RNA-DNA) and gRNA prodrugs. Currently, the major impurities observed in the synthesis of mlRNAs (e.g., a RNA that is 100 nucleotides in length) include (n−1)mers, (n−2)mers, (n−3)mers, (n−4)mers, (n−5)mers, (n+1)mers, and adducts. Methods, compositions and kits described herein can be used to synthesize and obtain highly purified mlRNAs (e.g., mlRNAs that are at least 80%, 85%, 90%, 95%, 99%, or more in purity).

### Splint Oligonucleotides

Disclosed herein include methods, compositions and kits for synthesizing RNAs, particularly mlRNAs such as gRNAs using splint-mediated ligation of two or more RNA fragments. In some embodiments, the method comprises use of a splint-mediated ligation of two, three, or more (e.g., four, five, six, seven, or eight) RNA fragments. The splint oligonucleotides can, for example, hybridize to a first RNA fragment and a second RNA fragment to form a complex, which facilitates ligation of the first and second RNA fragments at a ligation site present between the RNA fragments. In some embodiments, the method comprises use of a splint-mediated ligation of two RNA fragments using one splint oligonucleotide. In some embodiments, the method comprises use of a splint-mediated ligation of three RNA fragments using two splint oligonucleotides. In some embodiments, the method comprises use of a splint-mediated ligation of more than three RNA fragments (e.g., four, five, six, seven, or eight RNA fragments) using an appropriate number of splint oligonucleotides needed to ligate the RNA fragments.

It is appreciated in the art that it is very challenging to make gRNA with high purity using chemical synthesis. It is easier to purify shorter RNAs (e.g., RNAs with 30-40 nucleotides, including RNAs that are 34 or 35 nucleotides in length); however it is difficult to purify longer RNAs (e.g., RNAs longer than 90 nucleotides, including RNAs that are 99 or 100 nucleotides in length). In addition, it is easier to separate RNAs that differ by 30 or 60 nucleotides than separate RNAs that differ by a single or a few nucleotides. Major synthetic impurities overlap with Crispr region can results in reduced efficacy, off target effect(s), and/or induced toxicity. As described herein, in some embodiments (e.g., when the splint oligonucleotides are more than 32 nucleotides in length), after the splint-mediated ligation occurs, it can be challenging to obtain the synthesized mlRNAs (e.g., gRNAs) that are free from one or more of the splint oligonucleotides. Without being bound by any particular theory, in some embodiments it is expected that at least a portion of the mlRNAs synthesized from the ligation reaction remain bound to one or more splint oligonucleotides so that further process is needed to obtain free mlRNAs or mlRNAs in high purity. In some embodiments, when one splint oligonucleotide is used for ligating the first and second RNA fragments to synthesize mlRNAs, the splint oligonucleotide can remain bound to at least a portion of the synthesized mlRNAs. In some embodiments, when two splint oligonucleotides are used for ligating the first, second and third RNA fragments to synthesize mlRNAs, one or both of the two splint oligonucleotides can remain bound to at least a portion of the synthesized mlRNAs. The mlRNAs and the splint oligonucleotide(s) can be similar in melting temperature (Tm). The mlRNAs and the splint oligonucleotide(s) can be difficult to separate, in some embodiments, at high temperature including 50° C., 52° C., 54° C., 56° C., 58° C., 60° C., 61° C., 62° C., 63° C., 64° C., 65° C., 66° C., 67° C., 68° C., or a number or a range between any two of these values. Therefore, there can be a need for further process to separate the mlRNAs from the bound splint oligonucleotide(s) and thus obtain free mlRNAs and/or mlRNAs in high purity. As demonstrated herein, it can be advantageous to treating the ligated products with a DNase to digesting the splint oligonucleotide(s) and to obtain free mlRNAs and/or mlRNAs in high purity. As used herein, high purity can be, or be about, or be at least about, 80%, 85%, 90%, 91%, 92%, 93%, 94%, 95%, 96%, 96%, 97%, 98%, 99%, 99.5%, 100%, or a number or a range between any two of these values. The methods, compositions and kits disclosed herein can, for example, generate mlRNAs (e.g., gRNAs) in a purity of, or a purity of about, or a purity at least about, 80%, 85%, 90%, 91%, 92%, 93%, 94%, 95%, 96%, 96%, 97%, 98%, 99%, 99.5%, 100%, or a number or a range between any two of these values. Purified mlRNA (e.g., gRNA including sgRNA) generated by the methods, compositions, and kits described herein can be, be about, or be at least about, 75%, 80%, 85%, 90%, 91%, 92%, 93%, 94%, 95%, 96%, 96%, 97%, 98%, 99%, 99.5%, 100%, or a number or a range between any two of these values.

A splint oligonucleotide includes, for example, a first portion complementary to the first RNA fragment at the terminal region that includes a 5′ phosphate moiety. It further includes a second portion complimentary to the second RNA fragment at the terminal region comprising a 3′ hydroxyl group. The splint oligonucleotide can hybridize with the first RNA fragment and the second RNA fragment to form a complex. In the complex, the RNA fragments are positioned favorably for ligation at a ligation site present between the RNA fragments.

The splint oligonucleotide can include a sequence complementary to at least 1, 2, 3, 4, 5, 6, 7, 8, 9, 10, 11, 12, 13, 14, 15, 16, 17, 18, 19, 20, or a number or a range between any two of these values, nucleotides, consecutively or non-consecutively, in the first RNA fragment at the terminal region that includes a 5′ phosphate moiety. The splint oligonucleotide can include a sequence complementary to 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or a number or a range between any two of these values, nucleotides, of the first RNA fragment, and where the nucleotides can be consecutive or non-consecutive. The splint oligonucleotide can include a sequence complementary to at least 1, 2, 3, 4, 5, 6, 7, 8, 9, 10, 11, 12, 13, 14, 15, 16, 17, 18, 19, 20, or a number or a range between any two of these values, nucleotides, consecutively or non-consecutively, in the second RNA fragment at the terminal region comprising a 3′ hydroxyl group. The splint oligonucleotide may include a sequence complementary to 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or a number or a range between any two of these values, nucleotides, of the second RNA fragment, and where the nucleotides can be consecutive or non-consecutive. The length of the sequence in the splint oligonucleotide complementary to the first RNA fragment and the length of the sequence complementary to the second RNA fragment can be the same or can be different. In some embodiments, the length of the sequence in the splint oligonucleotide complementary to the first RNA fragment and the length of the sequence complementary to the second RNA fragment differ by, or by about, 1, 2, 3, 4, 5, 6, 7, 8, 9, 10, 11, 12, 13, 14, 15, 20, or a number or a range between any two of these values, nucleotides.

A splint oligonucleotide can be designed to preferentially promote complex formation between the RNA fragments and the splint oligonucleotide over intramolecular structures (e.g., secondary structures) present in the RNA fragments and/or the splint oligonucleotide. Minimum free energy prediction algorithms can be used for designing suitable splint oligonucleotides provided by the methods of the present disclosure. In theory, the lower the free energy, the more likely the complex between the RNA fragments and the splint oligonucleotide will form. The minimum free energy structure of a sequence is the secondary structure that is calculated to have the lowest value of free energy (and thus most likely to form in theory). By way of example, minimum free energy prediction algorithms can be used to calculate the free energy of the secondary structure(s) of an RNA fragment, which is represented by ΔGᵢₙₜᵣₐ, and the free energy of the intermolecular hybridization between the RNA fragment and the splint oligonucleotide, which is represented by ΔGᵢₙₜₑᵣ. In some embodiments, the Nearest-Neighbor approximations are used. The Tm of the secondary structure(s) of an RNA fragment is represented by Tₘ₋ᵢₙₜᵣₐ. The melting temperature of the RNA fragment and splint oligonucleotide hybrid is represented by Tₘ₋ᵢₙₜₑᵣ. The length of the splint oligonucleotide can be designed to ensure that ΔGᵢₙₜᵣₐ is greater than ΔGᵢₙₜₑᵣ, and/or Tₘ₋ᵢₙₜₑᵣ is greater than Tₘ₋ᵢₙₜᵣₐ. In instances where the first RNA fragment, the second RNA fragment, or both, comprises at least one secondary structure, hybridizing the first RNA fragment, the second RNA fragment, and the splint oligonucleotide results in a lower free energy than the free energy associated with one or more of the at least one secondary structure. Hybridizing the first RNA fragment, the second RNA fragment, and the splint oligonucleotide can also result in, for example, a lower free energy than that of the secondary structure with the lowest free energy (or the minimum free energy) of the first RNA fragment, the second RNA fragment, or both.

One or more splint oligonucleotides can be used to hybridize with the RNA fragments to mediate ligation of the RNA fragments. The number of splint oligonucleotide(s) used for mediating ligation can be fewer than the number of RNA fragments to be ligated. For example, the number of the splint oligonucleotide(s) used for mediating ligation can be one fewer than the number of RNA fragments to be ligated, i.e., if the number of RNA fragments to be ligated is n, the number of the splint oligonucleotide(s) can be n−1.

The length of the splint oligonucleotide can vary. For example, the splint oligonucleotide can be, or be about, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, or a number or a range between any two of these values, nucleotides in length.

In some embodiments, the splint oligonucleotides are more than 32 nucleotides in length. For example, the splint oligonucleotides are 33, 34, 35, 36, 38, 40, 45, 50, 55, 60, 65, 70, 75, 80, or a number of a range between any two of these values, nucleotides in length. In these embodiments, a portion of the mlRNAs (e.g., gRNAs) synthesized from a ligation reaction may remain bound to one or more splint oligonucleotides such that further process is needed to remove the bound splint oligonucleotides in order to obtain free mlRNAs and/or mlRNAs in high purity. For example, in these embodiments, the ligation reaction can be treated with a nuclease (e.g., DNase) to digest the splint nucleotide(s) and to obtain free mlRNAs and/or mlRNAs in high purity.

In some embodiments, the splint oligonucleotides are no more than 32 nucleotides in length. For example, the splint oligonucleotides are 10, 11, 12, 13, 14, 15, 16, 17, 18, 19, 20, 21, 22, 23, 24, 25, 26, 27, 28, 29, 31, or 32 nucleotides in length. In some embodiments, the splint oligonucleotides are short oligonucleotides. Short splint oligonucleotides are equal to or greater than 27 and less than 32 nucleotides in length. For example, the splint oligonucleotides are 27, 28, 29, 30, 31 or 32 nucleotides in length. In some embodiments, the splint oligonucleotides are very short oligonucleotides. Very short splint oligonucleotides are equal to or greater than 20 and less than 27 nucleotides in length. For example, the splint oligonucleotides are 10, 11, 12, 13, 14, 15, 16, 17, 18, 19, 20, 21, 23, 24, 25, or 26 nucleotides in length.

When the splint oligonucleotides are no more than 32 nucleotides in length (e.g., short or very short splint oligonucleotides), as described herein, the mlRNAs synthesized from the ligation reaction do not remain bound to one or more splint oligonucleotides (e.g., due to their low Tm values) so that no further process (e.g., enzymatic treatment) is needed to obtain free mlRNAs or mlRNAs in high purity. Examples of gRNA synthesis mediated by short or very short splint oligonucleotides demonstrate that short and very short splints mediated ligation can significantly improve the purity and yield of synthesized free mlRNAs (e.g., gRNAs) in large scale (e.g., 1 mg-5 mg) without the need of DNase treatment (see e.g., Example 5). In these embodiments, since the integrity of the splint oligonucleotides are maintained, the splint oligonucleotides can be isolated and further purified from the synthesized mlRNAs and/or unreacted RNA fragments and recycled for a next ligation cycle. By recycling the splint oligonucleotides, the method herein described can provide an advantage in reducing cost and time required to prepare new splint oligonucleotides.

In some embodiments, the splint oligonucleotide(s) is designed with a length sufficient to obtain a complex formed by the gRNA and the splint oligonucleotide(s) having a melting temperature (Tm) less than 60° C. For example, the Tm of the gRNA/splint DNA complex can be about, at most, or at most about 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., or a number or a range between any two of these values. In some embodiments, the splint oligonucleotides are designed with a length sufficient to obtain a Tm of the gRNA and/or the splint DNA greater than the temperature the RNA fragment ligation reaction is carried out. For example, the Tm of the gRNA and/or the splint DNA can be about, at least, or at least about 15° C., 16° C., 17° C., 18° C., 19° C., 20° C., 21, 22° C., 23° C., 24° C., 25° C., 26° C., 27° C., 28° C., 29° C., 30° C., 31° C., 32° C., 33° C., or a number or a range between any two of these values. In some embodiments, the Tm of the formed gRNA and DNA heteroduplex is greater than 15° C. and lower than 60° C. In some embodiments, the Tm of the formed gRNA and DNA heteroduplex is lower than 55° C., 50° C., 45° C., 40° C., 35° C. or 30° C.

The splint oligonucleotide can be in a free form, or be attached to a support. The splint oligonucleotide can be attached to the support using a variety of techniques. For example, the splint oligonucleotide can be directly attached to a support, or immobilized to the support by chemical immobilization. For example, a chemical immobilization can take place between functional groups on the support and corresponding functional elements in the splint oligonucleotide. Such corresponding functional elements in the splint oligonucleotide can either be an inherent chemical group of the splint oligonucleotide, e.g. a hydroxyl group or be additionally introduced. An example of such a functional group is an amine group. In some embodiments, the splint oligonucleotide is immobilized includes a functional amine group or is chemically modified to include a functional amine group.

The location of the functional group within the splint oligonucleotide to be immobilized can be used to control and shape the binding behavior and/or orientation of the splint oligonucleotide, e.g., the functional group can be placed at the 5′ or 3′ end of the splint oligonucleotide or within the sequence of the splint oligonucleotide. A typical support for a splint oligonucleotide to be immobilized includes moieties which are capable of binding to such splint oligonucleotide, e.g., to amine-functionalized nucleic acids. Non-limiting examples of such supports include carboxy, aldehyde, and epoxy supports. Supports on which a splint oligonucleotide is immobilized can be chemically activated, e.g. by the activation of functional groups, available on the support. The term “activated substrate” relates to a material in which interacting or reactive chemical functional groups were established or enabled by chemical modification procedures. For example, a support including carboxyl groups can be activated before use. Furthermore, certain supports contain functional groups that can react with specific moieties already present in the splint oligonucleotide.

A covalent linkage used to couple a splint oligonucleotide to a support can be viewed as both a direct and indirect linkage, in that although the splint oligonucleotide is attached by a “direct” covalent bond, there can be a chemical moiety or linker separating the “first” nucleotide of the splint oligonucleotide from the support, i.e., an indirect linkage. In some embodiments, splint oligonucleotides that are immobilized to the support by a covalent bond and/or chemical linker are generally seen to be immobilized or attached directly to the support. A splint oligonucleotide may not bind directly to the support, but interacts indirectly, for example by binding to a molecule which itself binds directly or indirectly to the support. The splint oligonucleotide can also be indirectly attached to a support (e.g., via a solution including a polymer).

In embodiments where the splint oligonucleotide is immobilized on the support indirectly, e.g., via hybridization to a surface oligonucleotide capable of binding the splint oligonucleotide, the splint oligonucleotide can further include an upstream sequence (5′ to the sequence that hybridizes to the two or more RNA fragments as described herein) that is capable of hybridizing to the 5′ end of the surface oligonucleotide. The splint oligonucleotide can be, for example, attached to the support via its 5′ end or its 3′ end. The splint oligonucleotide attached to the support can be in situ synthesized on the support.

### Methods of Synthesizing RNAs

Methods of synthesizing mlRNAs can include providing a first RNA fragment, a second RNA fragment, and a splint DNA oligonucleotide. The first RNA fragment, the second RNA fragment, and the splint oligonucleotide are hybridized together to form a complex. Forming such a complex positions the first and second RNA fragments in close proximity to facilitate ligation. A ligase is used to ligate the first and second RNA fragments across the ligation site to obtain an mlRNA. In some embodiments, a nuclease (e.g., DNase) is used to digest the splint DNA oligonucleotide thereby obtain an mlRNA. In some embodiments, the method does not comprise the use of a nuclease (e.g., a DNase).

In some embodiments, the methods comprise providing a first RNA fragment, a second RNA fragment, a third RNA fragment, a first splint DNA oligonucleotide, and a second DNA oligonucleotide. The first RNA fragment, the second RNA fragment, and the first splint oligonucleotide are hybridized together; and the second RNA fragment, the third RNA fragment, and the second splint oligonucleotides are hybridized together, thereby forming a complex comprising the first, second, and third RNA fragments, and the first and second splint DNA oligonucleotides. Formation of the complex positions (i) a 3′ hydroxyl group of the first RNA fragment and a 5′ phosphate moiety of the second RNA fragment in close proximity to provide a first ligation site; and (ii) a 3′ hydroxyl group of the second RNA fragment and a 5′ phosphate moiety of the third RNA fragment in close proximity to provide a second ligation site. The method further comprises using a ligase to ligate the first and second RNA fragments at the first ligation site, and to ligate the second and third RNA fragments at the second ligation site.

In some embodiments, the method further comprises using a DNase to digest the first and/or second splint DNA oligonucleotide(s) thereby synthesizing an mlRNA. In these embodiments, at least a portion of the mlRNAs synthesized from the ligation reaction may remain bound to one or more splint oligonucleotides so that further DNase treatment is needed to digest the splint oligonucleotides and to obtain free mlRNAs and/or mlRNAs in high purity. In some of these embodiments, the one or more splint oligonucleotide(s) is more than 32 nucleotides in length. In some embodiments, the one or more splint oligonucleotide(s) is no more than 32 nucleotides in length.

In some embodiments, the one or more splint oligonucleotide(s) is no more than 32 nucleotides in length and the method does not comprise using a DNase to digest the splint DNA oligonucleotide(s) (e.g., the first and/or second splint oligonucleotide(s)). For example, the method does not comprise a DNase treatment to digest the splint oligonucleotide(s) after ligation and before purification. The free mlRNAs and/or mlRNAs in high purity can be synthesized by using a ligase to ligate the first and second RNA fragments at the first ligation site, and to ligate the second and third RNA fragments at the second ligation site. In some embodiments, the mlRNAs synthesized from the ligation reaction can dissociate from the one or more splint oligonucleotides that mediate the ligation reaction. Therefore, the method does not comprise separating the mlRNAs (e.g., gRNAs) and the splint DNA oligonucleotides (e.g., via enzyme treatment). For example, the method does not comprise digesting the splint DNA oligonucleotide in the complex after the ligating step and before a purification step. In some embodiments, the ligation reaction does not comprise a DNase and/or the method does not comprise adding a DNase into a ligation reaction mixture prior to or after the ligation reaction. In some of these embodiments, the splint oligonucleotide is no more than 26 nucleotides in length (e.g., very short splint oligonucleotides). In some of these embodiments, the splint oligonucleotide is equal to or greater than 27 nucleotides and less than 32 nucleotides in length (e.g. short splint oligonucleotides).

In the embodiments when the splint oligonucleotide is no more than 32 nucleotides in length, even without the DNase treatment after the ligation reaction the method can still provide mlRNAs (e.g., gRNAs) in high purity, for example in a purity of, or a purity of about, or a purity at least about, 80%, 85%, 90%, 91%, 92%, 93%, 94%, 95%, 96%, 96%, 97%, 98%, 99%, 99.5%, 100%, or a number or a range between any two of these values. Purified mlRNA (e.g., gRNA including sgRNA) generated by the methods, compositions, and kits described herein can be, be about, or be at least about, 75%, 80%, 85%, 90%, 91%, 92%, 93%, 94%, 95%, 96%, 96%, 97%, 98%, 99%, 99.5%, 100%, or a number or a range between any two of these values. In some embodiments, the mlRNAs (e.g., gRNAs) can be provided at least 85%, 90%, 95%, or 98% in purity by these methods described herein.

Hybridizing the first RNA fragment, the second RNA fragment, and the splint DNA oligonucleotide can be performed in a solution. In some embodiments, the hybridizing further comprises a third RNA fragment and a second splint DNA oligonucleotide, which is performed in the solution. When hybridizing in solution, the concentration of the first RNA fragment can be, e.g., about the same as the concentration of the second RNA fragment. In some embodiments, wherein the hybridization further comprises a third RNA fragment, the concentration of the first RNA fragment and the second RNA fragment are each about the same to the concentration of the third RNA fragment. Depending upon the methods, fragments, and splint oligonucleotide(s) employed, the concentration of the splint oligonucleotide in the solution can be about the same as, more than, or less than, the concentration of the first RNA fragment in the solution, or a concentration of the second RNA fragment in the solution. For example, the concentration of the splint oligonucleotide, the first RNA fragment, and the second RNA fragment can be about equal. In some embodiments, the method comprises a first, second, and third RNA fragment, and a first and second splint oligonucleotide, where the concentration of the first splint oligonucleotide, the concentration of the second splint oligonucleotide, the concentration of the first RNA fragment, the concentration of the second RNA fragment, and the concentration of the third RNA fragment in the solution are each about the same.

For hybridizing, in some embodiments, the RNA fragments and/or the splint oligonucleotide can be denatured, i.e., the intramolecular structures of the RNA fragments and/or the splint oligonucleotide are disrupted to allow for annealing between the RNA fragments and the splint oligonucleotide. Denaturing can be achieved, for example, by heating a solution containing the RNA fragments and the splint oligonucleotide to, to about, or to at least about, 37° C., 38° C., 39° C., 40° C., 42° C., 44° C., 46° C., 48° C., 50° C., 52° C., 54° C., 56° C., 58° C., 60° C., 65° C., 70° C., 75° C., 80° C., 85° C., 90° C., 95° C., 100° C., or a number or a range between any two of these values. In some embodiments, hybridizing does not include heating the solution.

In some embodiments, hybridizing includes cooling the solution to a temperature of, or of about, 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or a number or a range between any two of these values, after heating. For example, in some embodiments, hybridizing includes cooling the solution to about 37° C. after heating. Hybridizing can include cooling the solution to a temperature at which a ligase used in the presently described methods retains ligase activity sufficient to ligate the first and second RNA fragments, and/or to a temperature below the melting temperature of the complex formed by the RNA fragments and the splint oligonucleotide upon hybridization. In instances where hybridizing does not comprise heating the solution, hybridizing can be carried out at a temperature that is lower than the melting temperature of the complex formed by the RNA fragments and the splint oligonucleotide upon hybridization. Depending on the specific method being performed, cooling the solution after heating can include reducing the temperature of the solution at a constant rate or at an uncontrolled rate.

The methods described herein include ligating the first and second RNA fragments using a ligase at a ligation site. Ligating can include ligating the 5′ phosphate group at the terminal region of the first RNA fragment with the 3′ hydroxyl group at the terminal region of the second RNA fragment. Catalyzed by the ligase, the 5′ phosphate group and the 3′ hydroxyl group can react to form a phosphodiester bond. A ligation site can be the site at which the phosphodiester bond between the 5′ phosphate group and the 3′ hydroxyl group is formed. In some embodiments, the methods comprise ligating a first RNA fragment and a second RNA fragment at a first ligation site, and ligating the second RNA fragment and a third RNA fragment at a second ligation site. In some embodiments, the ligating comprises ligation of a 3′ hydroxyl group at the terminus of the first RNA fragment with a 5′ phosphate at the terminus of the second RNA fragment; and ligation of a 3′ hydroxyl group at the terminus of the second RNA fragment and a 5′ phosphate at the terminus of the third RNA fragment, each ligation resulting in formation of a phosphodiester bond.

Ligating the first and second RNA fragments can be carried out at a suitable temperature, for example a temperature of, or of about, 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., or a number or a range between any two of these values. For example, ligating the first and second RNA fragments can be carried out at 37° C. Ligating the first and second RNA fragments can be carried out for various duration, for example, a duration of, or of about, 0.1, 0.2, 0.3, 0.4, 0.5, 1, 1.5, 2, 3, 4, 5, 6, 7, 8, 9, 10, 12, 14, 16, 18, 20, 25, 30, 35, 40, 45, or a number or a range between any two of these values hours. In some embodiments, the temperature and/or reaction time used for the splint-mediated ligation reaction is independent of the number of RNA fragments used in the reaction, for example, a reaction temperature and/or reaction time suitable for a splint-mediated ligation reaction comprising two RNA fragments is suitable for a splint-mediated ligation reaction comprising three or more RNA fragments.

In some embodiments, it can be advantageous to quench the ligation reaction following synthesis of the gRNA. For example, a ligation reaction can be quenched using a protease or a chelating agent. Non-limiting examples of proteases include proteinase K. Non-limiting examples of chelating agents include EDTA and EGTA, or a combination of both.

In some embodiments, ligating the first and second RNA fragments further comprises using one or more crowding agents, including but not limited to, polyethylene glycol (PEG), Ficoll®, ethylene glycol, and dextran, or any combination thereof. In some embodiments, use of one or more crowding agents is suitable in a splint-mediated ligation reaction comprising two, three, or more RNA fragments. In some embodiments, hybridizing a first RNA fragment, a second RNA fragment and a splint DNA oligonucleotide is carried out in the presence of one or more RNase inhibitors. In some embodiments, ligating the first and second RNA fragments with a ligase at a ligation site present between the first and second RNA fragments is carried out in the presence of one or more RNase inhibitors. In some embodiments, hybridizing three or more RNA fragments and two or more splint DNA oligonucleotides is carried out in the presence of one or more RNase inhibitors. In some embodiments, ligating three or more RNA fragments with a ligase at two or more ligation sites is carried out in the presence of one or more RNase inhibitors. RNase inhibitors can be used to inhibit and control RNase contamination during the hybridization and/or ligation reaction. Any of a variety of RNase inhibitors known in the art can be used herein.

A variety of ligases can be used in the methods, compositions and kits described herein. For example, the ligase can comprise, or be, a T4 DNA ligase, T4 RNA ligase I, T4 RNA ligase II, RtcB ligase, T3 DNA ligase, T7 DNA ligase, Taq DNA ligase, PBCV-1 DNA ligase, thermostable DNA ligase (e.g., 5′AppDNA/RNA ligase), an ATP dependent DNA ligase, or a combination thereof. Combinations of any two or more such ligases can be used in some embodiments. In some embodiments, the ligase is a T4 RNA ligase II or a variant thereof. For example, the T4 RNA ligase II can be truncated and/or comprise a mutation. For example, the T4 RNA ligase II can comprise a K227Q mutation and/or a R55K mutation. In some instances, the T4 RNA ligase II can be both truncated and have a K227Q and/or a R55K mutation. Also useful in the presently described methods is a PBCV-1 DNA ligase (i.e., Chlorella virus DNA ligase; SplintR® ligase). In some instances, the ligase can be a DNA ligase (e.g., a 9° N® DNA ligase).

In some embodiments, three or more (e.g., three, four or five) RNA fragments can be ligated to synthesize an mlRNA. Ligation of the three or more RNA fragments can be carried out in the same step, or in separate steps (such as in a step-wise fashion).

Methods described herein can further include separating the splint oligonucleotide(s), isolating the mlRNA, and/or purifying the mlRNA after synthesis. The isolation/purification can separate the full-length RNA product from unreacted RNA fragments and/or splint oligonucleotides. The isolation/purification can include, for example, enzymatically degrading the unreacted RNA fragments, e.g., using an exonuclease, such as one specific for 5′-monophosphate-containing RNA. An exemplary exonuclease is XRN-1.

In some embodiments, separating the splint oligonucleotide(s) is performed after the splint oligonucleotide(s) being treated with a DNase. Therefore, in some embodiments, the method described herein can include separating the digested splint oligonucleotide(s) after the splint oligonucleotide(s) being treated with the DNase.

In some embodiments, the method does not comprise treating the splint oligonucleotide(s) with the DNase. Therefore, in some embodiments, the method described herein can comprise separating the splint oligonucleotide and the mlRNAs (e.g., gRNAs) after ligating the RNA fragments (e.g., the first and second RNA fragments). The separated splint oligonucleotides and/or the mlRNAs can be isolated and/or purified. In these embodiments, since the splint oligonucleotides are not treated with any DNase, the integrity of the splint oligonucleotides are preserved. Therefore, in some embodiments, the method can include isolating and/or purifying the splint DNA oligonucleotide. The isolated and/or purified splint DNA oligonucleotide can be recycled and used for the next ligation reaction. Isolating and/or purifying the splint oligonucleotides can be carried out using any isolation and/or purification methods known in the art.

Purification of the full-length RNA product and/or splint oligonucleotides (e.g., from the unreacted RNA fragments) can be carried out using ultra-filtration or with chromatographic methods. Non-limiting examples of chromatographic methods include reversed-phase HPLC, ion-exchange chromatography (e.g., strong anion exchange HPLC or weak anion exchange HPLC), size exclusion chromatography, hydrophobic interaction chromatography, affinity chromatography, liquid chromatography-mass spectrometry (LCMS), capillary electrophoresis (CE), capillary gel electrophoresis (CGE), polyacrylamide gel purification, or any combination thereof.

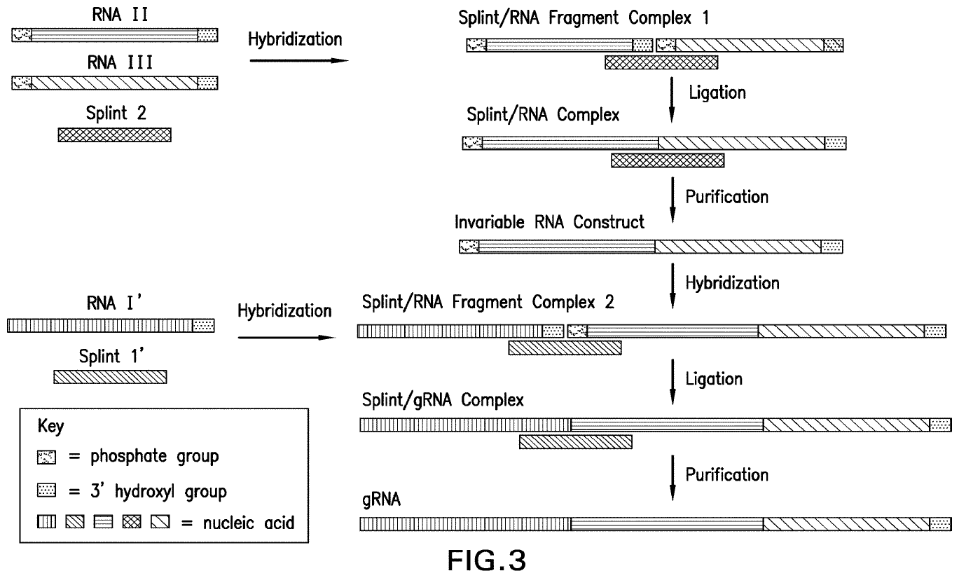

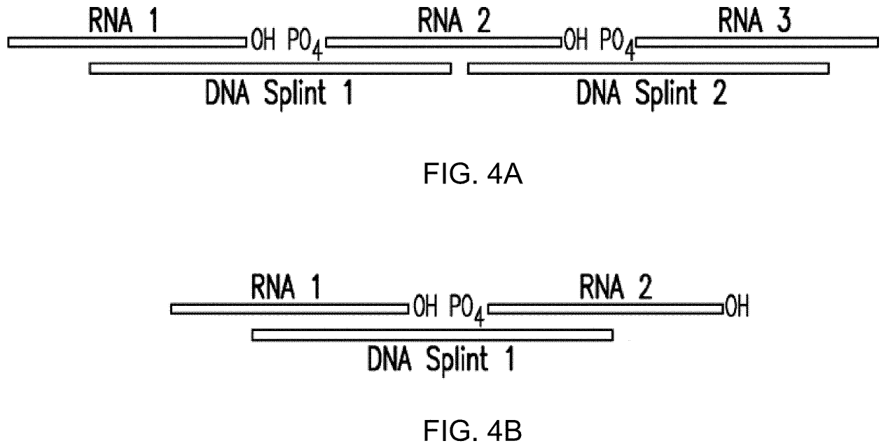

Also provided herein are methods of synthesizing a RNA molecule that includes all or a portion of a tracrRNA sequence, using any of the methods described herein. The RNA molecule can optionally be purified and used for generating a full-length gRNA (e.g., sgRNA). To generate a full-length sgRNA, an additional RNA fragment that includes a spacer and a minimum CRISPR repeat sequence (e.g., a crRNA) is ligated with the previously synthesized RNA molecule, using a splint oligonucleotide. These methods can, for example, produce full-length gRNAs specific for any target DNA sequence by ligating an RNA fragment containing the corresponding spacer sequence onto the previously synthesized RNA molecule comprising all or a portion of the tracrRNA sequence. FIG. 3 is a non-limiting schematic showing a three-fragment system where two of the RNA fragments (fragments II and III) are ligated first, using a splint oligonucleotide, to form an invariable RNA construct that includes a portion of a tracrRNA sequence. This RNA product can, for example, be isolated, purified and/or stored for later use. A RNA fragment I′ that includes a sequence complementary to a specific target DNA can then be combined with the previously synthesized RNA construct to form a full-length gRNA. In some embodiments, after ligation and before purification, DNase can be used to digest Splint 2, Splint 1, or both to facilitate isolation/purification of the gRNA (e.g., when one or more of the splint oligonucleotides is more than 32 nucleotides in length). In some embodiments, the method does not comprise a DNase treatment after ligation and before purification, while still capable of providing a full-length free gRNA and/or full-length gRNA in high purity (e.g., at least 80%, 85%, 90%, 95%, or 98% in purity), for example, when the one or more of the splint oligonucleotides is no more than 32 nucleotides in length (e.g., no more than 26 nucleotides in length).

### RNA Fragments

Method of synthesizing mlRNAs as described herein can include providing a first RNA fragment comprising a terminal region that includes a 5′ phosphate moiety, a second RNA fragment comprising a terminal region that includes a 3′ hydroxyl group, and a splint DNA oligonucleotide, where an mlRNA is synthesized by ligating the first and second RNA fragments. In some embodiments, the mlRNA is obtained by digesting the splint DNA oligonucleotide using a DNase. In some embodiments, the method of synthesizing mlRNAs does not comprise digesting the splint DNA oligonucleotide (e.g., using a DNase) after the ligation step. When synthesizing a gRNA, the first RNA fragment, the second RNA fragment, or both, can include at least a portion of a sequence that can bind to an RNA-guided endonuclease. An exemplary mlRNA can comprise, from 5′ to 3′, the second RNA fragment followed by the first RNA fragment. The second RNA fragment may not include a 5′ phosphate moiety. The 5′ phosphate moiety can be, e.g., a 5′-phosphate or a 5′-phosphorothioate. The first fragment, the second RNA fragment, or both, can include a sequence or a portion of a sequence that is complementary to a sequence in a target DNA. In some instances, the second RNA fragment comprises a sequence that is complementary to a sequence in a target DNA.

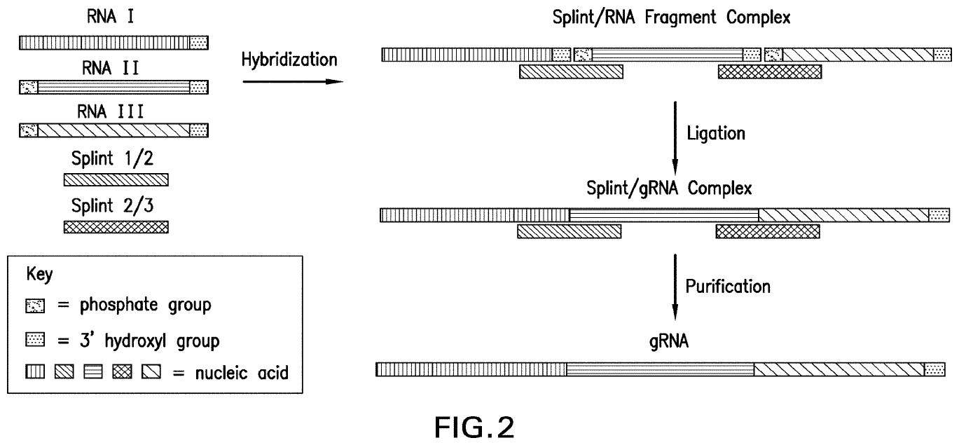

The mlRNAs can be synthesized by ligating three or more (e.g., three, four, five, or six) RNA fragments. FIG. 2 is a non-limiting schematic diagram showing the ligation of three RNA fragments using two splint oligonucleotides. As shown in FIG. 2, for an RNA synthesized by ligating RNA fragments I, II and III, prior to ligation, RNA fragment I can include a 3′ hydroxyl group and may or may not include a 5′ phosphate moiety, RNA fragment II can include both a 3′ hydroxyl group and a 5′ phosphate moiety, and RNA fragment III can include a 5′ phosphate moiety and may or may not include a 3′ hydroxyl group. Ligating RNA fragments I, II, and III can include formation of a phosphodiester bond between the 3′ hydroxyl group of RNA fragment I and the 5′ phosphate moiety of RNA fragment II, and a phosphodiester bond between the 3′ hydroxyl group of B and the 5′ phosphate moiety of RNA fragment III.

The length of the RNA fragments (e.g., the first, the second and the third RNA fragments) can vary. For example, the RNA fragment can be, or can be about, 10, 15, 20, 25, 30, 35, 40, 45, 50, 55, 60, 65, 70, 75, 80, 85, 90, or a number or a range of any two of these values, nucleotides in length. For example, the length of the first and second RNA fragments can be, for example, 10 to 90 nucleotides, each. The length of the second RNA fragment can be about 40 nucleotides or less (e.g. about 35, 30, 25, 20, 15, or about 10 nucleotides). In some embodiments, the length of the second RNA fragment can be about 20 nucleotides and the length of the first RNA fragment can be about 80 nucleotides. In some embodiments, the length of the second RNA fragment can be about 35 nucleotides and the length of the first RNA fragment can be about 65 nucleotides. In some embodiments, the length of the second RNA fragment can be about 40 nucleotides and the length of the first RNA fragment can be about 60 nucleotides.

A RNA fragment can include one or more secondary structures. The secondary structure of a RNA molecule (e.g., a RNA fragment or an mlRNA) can include stems and loops, or combination thereof. Non-limiting examples of secondary structures of an RNA molecule include stem loops, hairpin, hairpin loops, tetraloops, internal loops, bulges, pseudoknots, and cloverleaf. In some instances, an RNA fragment does not include any secondary structures (e.g., stem loops). The RNA synthesized by the methods provided herein can include one or more secondary structures, such as but not limited to, one or more stem loop structures, formed upon ligation of the RNA fragments. In some instances, the ligation site present between the RNA fragments correspond to a site in a secondary structure (e.g., a stem loop structure) in the synthesized RNA. The ligation site can correspond to a site in a portion of the secondary structure, including but not limited to, a tetraloop portion or a helix portion of a stem loop structure. The method disclosed herein can include predicting the secondary structure(s) of an RNA fragment and/or the free energy associated with the secondary structure(s), based on the sequence of the RNA fragment.

### Modifications in RNA Fragments

A RNA fragment can include one or more modifications. For example, the RNA fragment can include one or more modifications in the RNA backbone. Non-limiting examples of backbone modifications include: 2′ methoxy (2′OMe), 2′ fluorine (2′fluoro), 2′-O-methoxy-ethyl (MOE), locked nucleic acids (LNA), unlocked nucleic acids (UNA), bridged nucleic acids, 2′ deoxynucleic acids (DNA), and peptide nucleic acids (PNA). Alternatively or additionally, the RNA fragment can include one or more base modifications. Non-limiting examples of base modifications include: 2-aminopurine, hypoxanthine, thymine, 2,6-diaminopurine, 2-pyrimidone, and 5-methyl cytosine. In some embodiments, a RNA fragment comprises at least one phosphorothioate linkage.

Modifications in the RNA fragment can be used to, for example, enhance stability, reduce the likelihood or degree of innate immune response, and/or enhance other attributes; and new types of modifications are regularly being developed. Non-limiting examples of modification include one or more nucleotides modified at the 2′ position of the sugar, such as but not limited to, a 2′-O-alkyl, 2′-O-alkyl-O-alkyl, or 2′-fluoro-modified nucleotide. DNA (2′deoxy-) nucleotide substitutions are also contemplated. Non-limiting examples of RNA modifications also include 2′-fluoro, 2′-amino, 2′ O-methyl modifications on the ribose of pyrimidines, and basic residues or an inverted base at the 3′ end of the RNA. Such modifications can be incorporated into oligonucleotides, and these oligonucleotides have been shown to have a higher Tm (e.g., higher target binding affinity) than 2′-deoxy oligonucleotides against a given target. In some embodiments, the modification of an RNA fragment disclosed herein comprises a 2′O-methyl modification of one or more nucleosides in the RNA fragment.

The RNA fragment can include one or more modifications that increase resistance to nuclease digestion as compared to the native nucleic acid. In some embodiments, the modified nucleic acid comprises a modified backbone selected from, for example, phosphorothioates, phosphotriesters, methyl phosphonates, short chain alkyl or cycloalkyl intersugar linkages, and short chain heteroatomic or heterocyclic intersugar linkages. The nucleic acid can have a phosphorothioate backbone or a heteroatom backbone, e.g., CH₂—NH—O—CH₂, CH, —N(CH₃)—O—CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones; amide backbones; morpholino backbone structures; peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone. Phosphorus-containing linkages include, but are not limited to, phosphorothioates, chiral phosphorothioates, phosphorodithioates, phosphotriesters, aminoalkylphosphotriesters, methyl and other alkyl phosphonates including 3′alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. In some embodiments, the modification of the RNA fragment comprises one or more phosphorotioate linkages.

The RNA fragment can have a backbone that does not include a phosphorus atom, e.g., backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

The RNA fragment can comprise one or more substituted sugar moieties including, one of the following at the 2′ position: OH, SH, SCH₃, F, OCN, OCH₃, OCH₃O(CH₂)ₙ CH₃, O(CH₂)ₙ NH₂, or O(CH₂)ₙ, CH₃, where n is from 1 to 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. For example, a modification can include 2′ methoxyethoxy (2′-O—CH₂CH₂OCH₃, also known as 2′-O-(2-methoxyethyl)). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group. In some instances, both a sugar and an internucleoside linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a PNA. In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. The RNA fragment can include 2′-O-thionocarbamates MP (2′-O-methyl-3′-phosphonoacetate) and/or MSP (O-methyl-3′-thiophosphonoacetate).

The RNA fragment can include one or more modifications selected from pseudouridine, N′-methylpseudouridine, and 5-methoxyuridine. For example, one or more N′-methylpseudouridines can be incorporated into the RNA fragment to provide enhanced RNA stability and reduced immunogenicity in animal cells, such as mammalian cells (e.g., cells of human and mice). N′-methylpseudouridine modifications can also be incorporated in combination with one or more 5-methylcytidines.

The RNA fragment can include modifications designed to bypass innate antiviral responses and/or to reduce innate immune stimulation. For example, the RNA can be an enzymatically synthesized RNA incorporating 5′-Methylcytidine-5′-triphosphate (5-methyl-CTP), N6-methyl-ATP, pseudo-UTP, 2-thio-UTP, pseudoUTP, an Anti-Reverse Cap Analog (ARCA), or a combination thereof. The RNA fragment can include one or more modifications to enhance RNA stability, reduce innate immune responses, and/or achieve other benefits.

### Mimetics

The RNA fragment can be a nucleic acid mimetic. The term “mimetic” as it is applied to polynucleotides is intended to include polynucleotides wherein only the furanose ring or both the furanose ring and the internucleotide linkage are replaced with non-furanose groups. Replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone. In some embodiments, the RNA fragment is a PNA.

The RNA fragment can be a polynucleotide mimetic based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. Morpholino-based polynucleotides are nonionic mimics of oligonucleotides, which are less likely to form undesired interactions with cellular proteins. A variety of compounds within the morpholino class of polynucleotides have been prepared, having a variety of different linking groups joining the monomeric subunits.

The RNA fragment can be a polynucleotide mimetic referred to as cyclohexenyl nucleic acid (GeNA), where the furanose ring normally present in a DNA/RNA molecule is replaced with a cydohexenyl ring. GeNA DMT protected phosphoramidite monomers have been prepared and used for oligomeric compound synthesis following classical phosphoramidite chemistry.

The RNA fragment can be or comprise a locked nucleic acid (LNA), in which the 2′-hydroxyl group is linked to the 4′ carbon atom of the sugar ring, forming a 2′-C,4′-C-oxymethylene linkage, thereby forming a bicyclic sugar moiety. The linkage can be a methylene (—CH2-)ₙ group bridging the 2′ oxygen atom and the 4′ carbon atom wherein n is 1 or 2. LNA and LNA analogs display very high duplex thermal stabilities with complementary DNA and RNA (Tₘ=+3 to +10° C.), stability towards 3′-exonucleolytic degradation, and good solubility properties.

### Modified Sugar Moieties

An RNA fragment can include one or more substituted sugar moieties including, for example, a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH₂)ₙO)ₘCH₃, O(CH₂)ₙOCH₃, O(CH₂)ₙNH₂, O(CH₂)CH₃, O(CH₂)ₙONH₂, and O(CH₂)ₙON((CH₂)ₙCH₃)₂, where n and m are from 1 to about 10. Other RNA fragments include a suitable sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, a group for improving the pharmacodynamic properties of an oligonucleotide, and other substituents having similar properties. A suitable modification includes 2′-methoxyethoxy 2′-O—CH₂—CH₂OCH₃, also known as -2′-O-(2-methoxyethyl) or 2′-MOE) (Martin et al. (1995) Helv. Chim. Acta, 78(2):486-504) e.g., an alkoxyalkoxy group. A further suitable modification includes 2′-dimethylaminooxyethoxy, e.g., a O(CH₂)₂ON(CH₃)₂ group, also known as 2′-DMAOE, as described in examples herein below, and 2′ dimethylaminoethoxyethoxy (also known in the art as 2′-O-dimethyl-amino-ethoxy-ethyl or 2′ DMAEOE), e.g., 2′-O—CH₂—O—CH₂—N(CH₃)₂.

Other suitable sugar substituent groups include methoxy (—O—CH₃), aminopropoxy (—O—CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

The RNA fragment can include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. A “universal” base known in the art, e.g. hypoxanthine, can also be included. 5-Me-C substitutions have been shown to increase nucleic acid duplex stability by 0.6-1.2° C. and are embodiments of base substitutions.

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other a-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

The RNA fragment can include nucleobases for increasing the binding affinity. These include 5-substituted pyrimidines, 6-azapyrimidines and N-2, N-6 and —O-6 substituted purines, including 2-aminopropyladenine, 5-propynyluracil and 5-propynylcytosine. 5-methylcytosine substitutions have been shown to increase nucleic acid duplex stability by 0.6-1.2 oc.

The RNA fragment can comprise nucleobase modifications or substitutions may not have all positions uniformly modified. For example, an RNA fragment can have a modification incorporated in a single nucleoside.

### Synthesis of RNA Fragments and Splint Oligonucleotides

The RNA fragments and splint oligonucleotides provided by the present disclosure can be synthesized by any method suitable for oligonucleotide synthesis described herein or known in the art. Non-limiting examples include enzymatic synthesis and chemical synthesis (e.g., phosphoramidite chemistry). Methods of synthesizing RNA from a DNA template are known in the art. For example, the RNA fragments and splint oligonucleotides can be synthesized in vitro using an RNA polymerase enzyme (e.g., T7 polymerase, T3 polymerase, SP6 polymerase, etc.). Solid-phase synthesis using phosphoramidite chemistry involves assembling monomers of protected 2′-deoxynucleosides (dA, dC, dG, and T), ribonucleoside (A, C, G, and U), or chemically modified nucleosides, e.g., LNA or BNA. The monomers are sequentially coupled to the growing oligonucleotide chain in the order required by the sequence of the product. Upon the completion of the chain assembly, the product is released from the solid phase to solution, deprotected, and collected.

The RNA fragments and splint oligonucleotides can be synthesized in a 5′ to 3′ direction or a 3′ to 5′ direction. In some instances, the second RNA fragment is synthesized in a 5′ to 3′ direction. The synthesized RNA fragments and splint oligonucleotides may be purified prior to ligation in accordance with known methods in the art, such as, but not limited to: high-performance liquid chromatography (HPLC), reversed-phase HPLC, ion-exchange chromatography, size exclusion chromatography, hydrophobic interaction chromatography, affinity chromatography, and polyacrylamide gel purification.

### Moderate Length RNAs

An exemplary mlRNA synthesized using any of the methods described herein can be an be a guide RNA (gRNA). A gRNA can be a single gRNA (sgRNA). A gRNA can comprise a CRISPR RNA (crRNA) or a trans-activating crRNA (tracrRNA). A gRNA provides target specificity by virtue of its association with the RNA-guided endonuclease, and thus directs the activity of the RNA-guided endonuclease. RNAs of the present disclosure can be synthesized from two or more RNA molecules (termed RNA fragments) using one or more splints. An exemplary double-molecule gRNA comprises a crRNA and a transactivating crRNA (tracrRNA), and the crRNA and tracrRNA hybridize to each other to form a duplex. A double-molecule gRNA can also be a duplex of two crRNAs. The gRNA duplex can bind a RNA-guided endonuclease such that the gRNA and the RNA-guided endonuclease form a complex. A crRNA comprises both a spacer sequence capable of hybridizing to a target nucleic acid sequence of interest and a crRNA repeat sequence. TracrRNAs can be in any form (e.g., full-length tracrRNAs or active partial tracrRNAs) and of varying lengths. For example, a tracrRNA may comprise or consist of all or a portion of a wild-type tracrRNA sequence (e.g., about or at least 20, 26, 32, 45, 48, 54, 63, 67, 85 or more nucleotides of a wild-type tracrRNA sequence). Examples of wild-type tracrRNA sequences from S. pyogenes include the 171-nucleotide, 89-nucleotide, 75-nucleotide, and 65-nucleotide versions. As an example, the crRNA can have, in the 5′ to 3′ direction, an optional spacer extension sequence, a spacer sequence, and a minimum CRISPR repeat sequence. The tracrRNA can have a minimum tracrRNA sequence (complementary to the minimum CRISPR repeat sequence), a 3′ tracrRNA sequence, and an optional tracrRNA extension sequence. The optional tracrRNA extension may have elements that contribute additional functionality (e.g., stability) to the gRNA, and can have one or more hairpin structures. The crRNA and the tracrRNA hybridize through the minimum CRISPR repeat sequence and the minimum tracrRNA sequence to form a gRNA.

An exemplary sgRNA comprises a nucleotide sequence that is complementary to a sequence in a target DNA, and a nucleotide sequence that can bind to an RNA-guided endonuclease. As an example, an sgRNA can have, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence, and an optional tracrRNA extension sequence. In some instances, an sgRNA can have, in the 5′ to 3′ direction, a minimum CRISPR repeat sequence and a spacer sequence. The single-molecule guide linker links the minimum CRISPR repeat and the minimum tracrRNA sequence to form a hairpin structure. In some embodiments, the single-molecule guide linker is a tetraloop.

A CRISPR repeat sequence can include any sequence that has sufficient complementarity with a tracr sequence to promote one or more of: (1) excision of a DNA targeting segment flanked by CRISPR repeat sequences in a cell containing the corresponding tracr sequence; and (2) formation of a CRISPR complex at a target sequence, wherein the CRISPR complex includes the CRISPR repeat sequence hybridized to the tracr sequence. In general, degree of complementarity is with reference to the optimal alignment of the CRISPR repeat sequence and tracr sequence, along the length of the shorter of the two sequences. Optimal alignment may be determined by any suitable alignment algorithm and may further account for secondary structures, such as self-complementarity within either the tracr sequence or CRISPR repeat sequence. In some instances, the degree of complementarity between the tracr sequence and CRISPR repeat sequence along the 30 nucleotides length of the shorter of the two when optimally aligned is about or more than 25%, 30%, 40%, 50%, 60%, 70%, 80%, 90%, 95%, 97.5%, 99%, or higher. The tracr sequence can be, or be about, 5, 6, 7, 8, 9, 10, 11, 12, 13, 14, 15, 16, 17, 18, 19, 20, 25, 30, 40, 50, or a number or a range between any two of these values, nucleotides in length.

The spacer of a gRNA includes a nucleotide sequence that is complementary to a sequence in a target DNA. In other words, the spacer of a gRNA interacts with a target DNA in a sequence-specific manner via hybridization (e.g., base pairing). As such, the nucleotide sequence of the spacer may vary and determines the location within the target DNA that the gRNA and the target DNA will interact. The spacer of a gRNA can be selected to hybridize to any desired sequence within a target DNA.

The spacer can have a length of 10, 11, 12, 13, 14, 15, 16, 17, 18, 19, 20, 21, 22, 23, 24, 25, 26, 27, 28, 29, 30, or a number or a range between any two of these values, nucleotides. For example, the spacer can have a length of from 13 to 25 nucleotides, from 15 to 23 nucleotides, from 18 to 22 nucleotides, or from 20 to 22 nucleotides.

The percent sequence complementarity between the spacer of a gRNA and a target sequence of a target DNA can be, e.g., at least about 60% (such as at least about any of 65%, 70%, 75%, 80%, 85%, 90%, 95%, 97%, 98%, 99%, or 100%).

The length of the gRNA synthesized by the methods described herein can be from 30 to 160 nucleotides, including 30, 35, 40, 45, 50, 55, 60, 65, 70, 75, 80, 85, 90, 95, 100, 105, 110, 115, 120, 125, 130, 135, 140, 145, 150, 155, 160, or a number or a range between any two of these values, nucleotides. The gRNA synthesized by the methods described herein can include a spacer. In some embodiments, the gRNA includes a sequence that is complementary to a sequence in a target DNA, including but not limited to, a target mammalian DNA. For example, the target DNA can be human DNA.

Modifications in the mlRNAs

mlRNAs as described herein can include one or more modifications useful for e.g., tracking, increasing stability, targeting the RNA to a particular subcellular location, or reducing immunogenicity. Modifications of gRNAs can be used to enhance the formation or stability of a DNA-editing complex comprising a gRNA and an RNA-guided endonuclease (e.g., a Cas endonuclease, such as a Cas9 endonuclease). Modifications of gRNAs can also or alternatively be used to enhance the initiation, stability, or kinetics of interactions between a DNA-editing complex and a target sequence in a target DNA, which can be used, for example, to enhance on-target activity. Modifications of gRNAs can also or alternatively be used to enhance specificity, e.g., the relative rates of DNA editing at an on-target site as compared to effects at other (off-target) sites. Modifications can also or alternatively be used to increase the stability of an RNA, e.g., by increasing its resistance to degradation by ribonucleases (RNases) present in a cell, thereby causing its half-life in the cell to be increased.

The mlRNAs can include a segment at either the 5′ or 3′ end that provides for any of the features described above. For example, a suitable segment can include a riboswitch sequence (e.g., to allow for regulated stability and/or regulated accessibility by proteins and protein complexes); a stability control sequence; a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides for responses to light or radiation (e.g., UV, vis, IR optogenetic elements); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); a modification or sequence that provides for increased, decreased, and/or controllable stability; and combinations thereof.

The mlRNAs can include a modification that decreases the likelihood or degree to which the RNA, when introduced into a cell, elicits an innate immune response. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses. The mlRNAs can include one or more modifications selected from modifications that enhance the stability of the RNA (such as by decreasing its degradation by RNases, e.g., in the context of a cell) and modifications that decrease the likelihood or degree to which the RNA, when introduced into a cell, elicits an innate immune response. Combinations of modifications, such as the foregoing and others, can likewise be used.

### Stability Control Sequence

The mlRNAs can include a stability control sequence that influences the stability of the RNA. A non-limiting example of a suitable stability control sequence is a transcriptional terminator segment (e.g., a transcription termination sequence). A transcriptional terminator segment of an RNA can have a total length of from about 10 nucleotides to about 100 nucleotides, for example 10, 15, 20, 25, 30, 35, 40, 45, 50, 55, 60, 65, 70, 75, 80, 85, 90, 95, 100, or a number or a range between any two of these values, nucleotides in length.

The transcription termination sequence can be one that is functional in a eukaryotic cell, a prokaryotic cell, or both. Nucleotide sequences that can be included in a stability control sequence (e.g., transcriptional termination segment, or in any segment of the RNA to provide for increased stability) include, for example, a Rho-independent trp termination site.

### Conjugates

The mlRNAs can include a modification involving chemically linking to the gRNA one or more moieties or conjugates which enhance the activity, cellular distribution, or cellular uptake of the RNA. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid.

The mlRNAs can include a chemically linked conjugate moieties including, but not limited to, lipid moieties such as a cholesterol moiety; cholic acid; a thioether, e.g., hexyl-S-tritylthiol; a thiocholesterol; an aliphatic chain, e.g., dodecandiol or undecyl residues, a phospholipid, e.g. di-hexadecyl-rac-glycerol or triethylammonium 1,2-di-O-hexadecyl-rac-glycero-3-H phosphonate, a polyamine or a polyethylene glycol chain; adamantane acetic acid; a palmityl moiety; or an octadecylamine or hexylamino-carbonyl-t oxycholesterol moiety.

The mlRNAs can include a chemically linked conjugate including a “Protein Transduction Domain” or PTD (also known as a cell penetrating peptide, or CPP), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. A PTD can be covalently linked to a gRNA. Exemplary PTDs include, but are not limited to, a minimal undecapeptide protein transduction domain (corresponding to residues 47-57 of HIV-1 TAT); a polyarginine sequence comprising a number of arginines sufficient to direct entry into a cell (e.g., 3, 4, 5, 6, 7, 8, 9, 10, or 10-50 arginines); a VP22 domain; a Drosophila antennapedia protein transduction domain; a truncated human calcitonin peptide; and polylysine. The PTD can be an activatable CPP (ACPP) which includes a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane. The PTD can be chemically modified to increase the bioavailability of the PTD.

The mlRNAs can include an applied conjugate that can enhance its delivery and/or uptake by cells, including, for example, cholesterol, tocopherol and folic acid, lipids, peptides, polymers, linkers, and aptamers.

### RNA-Guided Endonucleases

As described herein, one or more of the first RNA fragment, the second RNA fragment, the third RNA fragment can comprise at least a portion of a sequence that can bind to an RNA-guided endonuclease. The RNA-guided endonuclease can be naturally-occurring or non-naturally occurring. Non-limiting Examples of RNA-guided endonuclease include a Cas1, Cas1B, Cas2, Cas3, Cas4, Cas5, Cash, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, and functional derivatives thereof. In some instances, the RNA-guided endonuclease is a Cas9 endonuclease. The Cas9 endonuclease can be from, e.g., Streptococcus pyogenes (SpyCas9), Staphylococcus lugdunensis (SluCas9), or Staphylococcus aureus (SaCas9). In some embodiments, the RNA-guided endonuclease is a variant of Cas9, including but not limited to, a small Cas9, a dead Cas9 (dCas9), and a Cas9 nickase.

The RNA-guided endonuclease can be a small RNA-guided endonuclease. The small RNA-guided endonucleases can be engineered from portions of RNA-guided endonucleases derived from any of the RNA-guided endonucleases described herein and known in the art. The small RNA-guided endonucleases can be, e.g., small Cas endonucleases. In some cases, a small RNA-guided nuclease is shorter than about 1,100 amino acids in length.

The RNA-guided endonuclease can be a mutant RNA-guided endonuclease. For example, the RNA-guided endonuclease can be a mutant of a naturally occurring RNA-guided endonuclease. The mutant RNA-guided endonuclease can also be a mutant RNA-guided endonuclease with altered activity compared to a naturally occurring RNA-guided endonuclease, such as altered endonuclease activity (e.g., altered or abrogated DNA endonuclease activity without substantially diminished binding affinity to DNA). Such modification can allow for the sequence-specific DNA targeting of the mutant RNA-guided endonuclease for the purpose of transcriptional modulation (e.g., activation or repression); epigenetic modification or chromatin modification by methylation, demethylation, acetylation or deacetylation, or any other modifications of DNA binding and/or DNA-modifying proteins known in the art. In some embodiments, the mutant RNA-guided endonuclease has no DNA endonuclease activity.

The RNA-guided endonuclease can be a nickase that cleaves the complementary strand of the target DNA but has reduced ability to cleave the non-complementary strand of the target DNA, or that cleaves the non-complementary strand of the target DNA but has reduced ability to cleave the complementary strand of the target DNA. In some embodiments, the RNA-guided endonuclease has a reduced ability to cleave both the complementary and the non-complementary strands of the target DNA.

Methods of Synthesizing gRNAs

In some embodiments, the disclosure provides methods for synthesizing a gRNA (e.g., sgRNA) for use with an RNA-guided endonuclease. The RNA-guided endonuclease can be, for example a Cas endonuclease, including Cas9 endonuclease. The Cas9 endonuclease can be, for example, a SpyCas9, a SaCas9, or a SluCas9 endonuclease. In some embodiments, the RNA-endonuclease is a Cas9 variant. In some embodiments, the RNA-guided endonuclease is a small RNA-guided endonuclease. In some embodiments, the RNA-guided endonuclease is a small Cas endonuclease.

In some embodiments, the gRNA comprise 5′ to 3′: a crRNA and a tracrRNA, wherein the crRNA and tracrRNA hybridize to form a duplex. In some embodiments, the crRNA comprises a spacer sequence capable of targeting a target sequence in a target nucleic acid (e.g., genomic DNA molecule) and a crRNA repeat sequence. In some embodiments, the tracrRNA comprises a tracrRNA anti-repeat sequence and a 3′ tracrRNA sequence. In some embodiments, the 3′ end of the crRNA repeat sequence is linked to the 5′ end of the tracrRNA anti-repeat sequence, e.g., by a tetraloop, wherein the crRNA repeat sequence and the tracrRNA anti-repeat sequence hybridize to form the sgRNA. In some embodiments, the sgRNA comprises 5′ to 3′: a spacer sequence, a crRNA repeat sequence, a tetraloop, a tracrRNA anti-repeat sequence, and a 3′ tracrRNA sequence. In some embodiments, the sgRNA comprise a 5′ spacer extension sequence. In some embodiments, the sgRNA comprise a 3′ tracrRNA extension sequence. The 3′ tracrRNA can comprise, or consist of, one or more stem loops, for example one, two, three, or more stem loops.

In some embodiments, the method comprises synthesizing the sgRNA using a splint-mediated ligation approach comprising two RNA fragments and one splint oligonucleotide. In some embodiments, the method comprises providing a complex formed between a first RNA fragment, a second RNA, and a splint oligonucleotide; and a ligase, wherein (a) the first RNA fragment comprises a terminal region comprising a 3′ hydroxyl group; (b) the second RNA fragment comprises a terminal region comprising a 5′ phosphate moiety; and (c) the splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and wherein the complex is formed by hybridization of (a) and (c)(i) and hybridization of (b) and (c)(ii), wherein the complex comprises a ligation site present between the 3′ hydroxyl group of the first RNA fragment and the 5′ phosphate group of the second RNA fragment, wherein the ligase results in a ligation at the ligation site to form a phosphodiester bond between the 3′hydroxyl group of the first RNA fragment and the 5′ phosphate group of the second RNA fragment, the ligation forming a sgRNA comprising from 5′ to 3′: a spacer sequence and an invariable sequence, the invariable sequence comprising a duplex formed between a crRNA repeat sequence and a tracrRNA anti-repeat sequence, and a 3′ tracrRNA sequence comprising at least one stem-loop, thereby synthesizing the sgRNA. In some embodiments, the ligation site corresponds to a site in the duplex formed between a crRNA repeat sequence and a tracrRNA anti-repeat sequence. In some embodiments, the ligation site is in the crRNA repeat sequence, in a tetraloop joining the crRNA repeat sequence and the tracRNA anti-repeat sequence, or in the tracrRNA anti-repeat sequence. In some embodiments, the ligation site is within a stem-loop of the 3′ tracrRNA sequence. In some embodiments, the first RNA fragment comprises the nucleotide sequence of the sgRNA that is 5′ the ligation site; and the second RNA fragment comprises the nucleotide sequence of the sgRNA that is 3′ the ligation site. In some embodiments, DNase treatment is conducted to digest the splint oligonucleotides to reduce or eliminate the duplex/complex formed between the splint oligonucleotide(s) and the sgRNA.

The method can comprise synthesizing a sgRNA using a splint-mediated ligation approach comprising three RNA fragments and two splint oligonucleotides. In some embodiments, the method comprises providing a complex formed between a first RNA fragment, a second RNA fragment, a third RNA fragment, first splint oligonucleotide, and a second splint oligonucleotide; and a ligase, wherein (a) the first RNA fragment comprises (i) a terminal region comprising a 3′ hydroxyl group; (b) the second RNA fragment comprises (i) a first terminal region comprising a 5′ phosphate moiety, and (ii) a second terminal region comprising a 3′ hydroxyl group; (c) the third RNA fragment comprises (i) a terminal region comprising a 5′ phosphate moiety; (d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and (e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment, wherein the complex is formed by hybridization of (a)(i) and (d)(i), (b)(i) and (d)(ii), (b)(ii) and (e)(i), and (c)(i) and (e)(ii), wherein the complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment, wherein the ligase results in a ligation at the first ligation site, forming a phosphodiester bond between the 3′ hydroxyl group of the first RNA fragment and the 5′ phosphate group of the second RNA fragment, and a ligation at the second ligation site, forming a phosphodiester bond between the 3′ hydroxyl group of the second RNA fragment and the 5′ phosphate group of the third RNA fragment, the ligation forming a sgRNA comprising from 5′ to 3′: a spacer sequence and an invariable sequence, the invariable sequence comprising a duplex formed between a crRNA repeat sequence and a tracrRNA anti-repeat sequence, and a 3′ tracrRNA sequence comprising at least one stem-loop, thereby synthesizing the sgRNA. In some embodiments, DNase treatment is conducted to digest the splint oligonucleotides to reduce or eliminate the duplex/complex formed between the first and/or the second splint oligonucleotide and the sgRNA.

In some embodiments when the one or more splint oligonucleotide(s) is more than 32 nucleotides in length, a portion of the gRNAs (e.g., sgRNAs) synthesized from the ligation can remain bound to the splint oligonucleotide(s) so that further DNase treatment is performed to digest the splint oligonucleotide(s) and to obtain free gRNAs and/or gRNAs in high purity.

In some embodiments, DNase treatment is not conducted in a method of synthesizing the sgRNA herein described. For example, in some embodiments, the splint oligonucleotide(s) is no more than 32 nucleotides in length (e.g., no more than 26 nucleotides in length) and the method does not comprise using a DNase to digest the one or more splint DNA oligonucleotide(s). For example, the method does not comprise a DNase treatment after the ligation step and before purification. In some embodiments, the sgRNAs synthesized from the ligation reaction can dissociate from the one or more splint oligonucleotides that mediate the ligation reaction. Therefore, the method does not comprise separating the sgRNAs and the splint DNA oligonucleotides (e.g., via enzyme treatment). For example, the method does not comprise digesting the splint DNA oligonucleotide in the complex after the ligating step and before a purification step. In some of these embodiments, the splint oligonucleotide is no more than 26 nucleotides in length (e.g., very short splint oligonucleotides). In some of these embodiments, the splint oligonucleotide is equal to or greater than 27 nucleotides and less than 32 nucleotides in length (e.g. short splint oligonucleotides).

In some embodiments, the ligation forming a sgRNA can be carried out at a suitable temperature, for example, a temperature of, or of about, 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., or a number or a range between any two of these values. For example, ligating the first and second RNA fragments can be carried out at 37° C.

In some embodiments, the ligation forming a sgRNA can be carried out at a temperature lower than 60° C., optionally, lower than 55° C., 50° C., 45° C., 40° C. or 35° C. For example, when the one or more splint oligonucleotide(s) used to ligate the RNA fragments is no more than 32 nucleotides in length (e.g., no more than 26 nucleotides in length), ligating the RNA fragments can be carried out at a temperature lower than 60° C., optionally, lower than 55° C., 50° C., 45° C., 40° C. or 35° C. In some embodiments, ligating two or more RNA fragments can be carried out at about 15° C. to about 33° C. In some embodiments, ligating the RNA fragments can be carried out at about, at least, at least about, at most or at most about 15° C., 16° C., 17° C., 18° C., 19° C., 20° C., 21, 22° C., 23° C., 24° C., 25° C., 26° C., 27° C., 28° C., 29° C., 30° C., 31° C., 32° C., 33° C., or a number or a range between any two of these values. For example, the ligation can be carried out at about 15° C., 20° C., 22° C., 24° C., 27° C., 30° C., or 33° C.

The ligation forming a sgRNA can be carried out for various duration, for example, a duration of, or of about, 0.1, 0.2, 0.3, 0.4, 0.5, 1, 1.5, 2, 3, 4, 5, 6, 7, 8, 9, 10, 12, 14, 16, 18, 20, 25, 30, 35, 40, 45, or a number or a range between any two of these values, hours. In some embodiments, ligating two or more RNA fragments can be carried out for several hours, for example, a duration of, or of about, 3, 4, 5, 6, 7, 8, 9, 10 or a number or a range between any two of these values, hours. In some embodiments, ligating two or more RNA fragments is carried out for about 6 hours.

In some embodiments, the temperature and/or reaction time used for the splint-mediated ligation reaction is independent of the number of RNA fragments used in the reaction, for example, a reaction temperature and/or reaction time suitable for a splint-mediated ligation reaction comprising two RNA fragments is suitable for a splint-mediated ligation reaction comprising three or more RNA fragments.

In some embodiments, the method can include separating the splint oligonucleotides and/or the sgRNAs after ligating the RNA fragments. The separated splint oligonucleotides and/or the sgRNAs can be isolated and/or purified in accordance to any method known in the art such as ultra-filtration and chromatographic methods.

In some embodiments, with the DNase treatment of the splint oligonucleotides, the obtained sgRNAs can be in a purity of, or a purity of about, or a purity at least about, 80%, 85%, 90%, 91%, 92%, 93%, 94%, 95%, 96%, 96%, 97%, 98%, 99%, 99.5%, 100%, or a number or a range between any two of these values.

In the embodiments when the splint oligonucleotide is no more than 32 nucleotides in length, even without the DNase treatment after the ligation reaction the method can provide sgRNAs in a purity of, or a purity of about, or a purity at least about, 80%, 85%, 90%, 91%, 92%, 93%, 94%, 95%, 96%, 96%, 97%, 98%, 99%, 99.5%, 100%, or a number or a range between any two of these values.

In some embodiments, the first ligation site corresponds to a site in the duplex formed between a crRNA repeat sequence and a tracrRNA anti-repeat sequence. In some embodiments, the first ligation site is in the crRNA repeat sequence, in a tetraloop joining the crRNA repeat sequence and the tracRNA anti-repeat sequence, or in the tracrRNA anti-repeat sequence.

In some embodiments, the 3′ tracrRNA sequence comprises a first, second, and third stem loop. In some embodiments, the second ligation site corresponds to a site in the first stem loop, the second stem loop, or the third stem loop. In some embodiments, the second ligation site corresponds to a site in the second stem loop. In some embodiments, the second ligation site corresponds to a site in the 5′ stem of the second stem loop, a site in the tetraloop of the second stem loop, or a site in the 3′ stem of the second stem loop. In some embodiments, the second ligation site is (i) immediately adjacent to the base of the 5′stem of the second stem loop; (ii) proximal to the base of the 5′stem of the second stem loop (e.g., ±1 nucleotides (nt), ±2 nt, or ±3 nt from the base of the 5′ stem); (iii) immediately adjacent to the base of the 3′ stem of the second stem loop; or (iv) proximal to the base of the 3′ stem of the second stem loop (e.g., ±1 nt, ±2 nt, or ±3 nt from the base of the 3′ stem).

In some embodiments, the first RNA fragment comprises the nucleotide sequence of the sgRNA that is 5′ the first ligation site; the second RNA fragment comprises the nucleotide sequence of the sgRNA that is 3′ the first ligation site and 5′ the second ligation site; and the third RNA fragment comprises the nucleotide sequence of the sgRNA that is 3′ the second ligation site. In some embodiments, the first RNA fragment comprises 5′ to 3′: the spacer sequence, and a portion of the crRNA repeat sequence of the sgRNA. In some embodiments, the first RNA fragment comprises 5′ to 3′: the spacer sequence, and the crRNA repeat sequence of the sgRNA. In some embodiments, the first RNA fragment comprises 5′ to 3′: the spacer sequence, the crRNA repeat sequence, and a portion of the tetraloop of the sgRNA. In some embodiments, the first RNA fragment comprises 5′ to 3′: the spacer sequence, the crRNA repeat sequence, and the tetraloop of the sgRNA. In some embodiments, the first RNA fragment comprises 5′ to 3′: the spacer sequence, the crRNA repeat sequence, the tetraloop, and a portion of the tracRNA repeat sequence of the sgRNA. In some embodiments, the first RNA fragment comprises 5′ to 3′: the spacer sequence, the crRNA repeat sequence, the tetraloop, and the tracRNA repeat sequence of the sgRNA. In some embodiments, the terminal region of (a)(i), which is complementary to (d)(i) of the first splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, 34, or a number or a range between any two of these values, nucleotides in length, wherein the nucleotide sequence is positioned at the 3′ end of the first RNA fragment. In some embodiments, the terminal region of (a)(i) extends from the 3′ terminus of the first RNA fragment to the 3′ terminus of the spacer sequence (e.g., wherein the 5′ terminus of the spacer sequence is aligned with the 5′ terminus of the first RNA fragment). In some embodiments, the terminal region of (a)(i) extends from the 3′terminus of the first RNA fragment to include the 1, 2, 3, 4, 5, 6, 7, 8, 9, 10, 11, 12, 13, 14, 15, 16, 17, 18, 19, or 20 nucleotides that are present at the 3′ end of the spacer sequence. In some embodiments, the first portion (d)(i) of the first splint oligonucleotide is perfectly complementary to the terminal region of (a)(i). In some embodiments, the first portion (d)(1) of the first splint oligonucleotide has 1, 2, or 3 mismatches relative to the terminal region of (a)(i).

In some embodiments, the second RNA fragment comprises 5′ to 3′: the crRNA repeat sequence, the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: a portion of the crRNA repeat sequence, the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: a portion of the tetraloop, the tracrRNA repeat sequence, and a portion of the 3′tracrRNA sequence (i.e., a portion that is 5′ to the second ligation site) of the sgRNA. In some embodiments, the second RNA fragment comprises 5′ to 3′: the tracrRNA repeat sequence, and a portion of the 3′tracrRNA (i.e., a portion that is 5′ to the second ligation site) of the sgRNA. In some embodiments, the second RNA fragment comprises 5′ to 3′: a portion of the tracrRNA repeat sequence, and a portion of the 3′tracrRNA (i.e., a portion that is 5′ to the second ligation site) of the sgRNA. In some embodiments, the terminal region (b)(i), which is complementary to the second portion (d)(ii) of the first splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, or 34 nucleotides in length and is located at the 5′end of the second RNA fragment. In some embodiments, the second portion (d)(ii) of the first splint oligonucleotide is perfectly complementary to the terminal region of (b)(i), or has 1, 2, or 3 mismatches relative to the terminal region of (d)(ii). In some embodiments, the terminal region (b)(ii) of the second RNA fragment, which is complementary to the first portion (e)(i) of the second splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, or 34 nucleotides in length and is located at the 3′end of the second RNA fragment. In some embodiments, the first portion (e)(i) of the second splint oligonucleotide is perfectly complementary to the terminal region of (b)(ii), or has 1, 2, or 3 mismatches relative to the terminal region of (b)(ii).

The third RNA fragment can comprise a portion of the 3′ tracrRNA sequence (i.e., that is 3′ to the second ligation site) of the sgRNA. In some embodiments, the terminal region (c)(i) of the third RNA fragment, which is complementary to the second portion (e)(ii) of the second splint oligonucleotide, comprises a nucleotide sequence that is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length and is located at the 5′end of the third RNA fragment. In some embodiments, the second portion (e)(ii) of the second splint oligonucleotide is perfectly complementary to the terminal region of (c)(i), or has 1, 2, or 3 mismatches relative to the terminal region of (c)(i).

In some embodiments, the invariable sequence of the sgRNA comprises the nucleotide sequence of GUUUUAGAGCUAGAAAUAGCAAGUUAAAAUAAGGCUAGUCCGUUAUCAACUUGAAA AAGUGGCACCGAGUCGGUGCUUUU (SEQ ID NO: 1), or a nucleotide sequence having up to 1, 2, 3, 4, 5, 6, 7, 8, 9, or 10 nucleotide deletions, insertions, or substitutions relative to SEQ ID NO: 1. In some embodiments, the sgRNA is for use with a SpyCas9 endonuclease.

As described herein, purified mlRNA (e.g., gRNA including sgRNA) can be, be about, or be at least about, 75%, 80%, 85%, 90%, 91%, 92%, 93%, 94%, 95%, 96%, 96%, 97%, 98%, 99%, 99.5%, 100%, or a number or a range between any two of these values.

In the methods described herein, hybridizing step can be carried out with the ligating step in some embodiments, e.g., hybridization carried out in the presence of the ligase. In some embodiments, hybridizing step starts before the ligating step or occurs (e.g., completed) before the ligating step, and optionally the hybridizing step can occur at a different temperature than the ligation step.

### EXAMPLES

Some aspects of the embodiments discussed above are disclosed in further detail in the following examples, which are not in any way intended to limit the scope of the present disclosure.

### Example 1

### gRNA Synthesis by Splint-Mediated Ligation and DNase Treatment

This example demonstrates that full-length gRNA molecules can be generated efficiently and in high purity using splint-mediated ligation and DNase treatment.

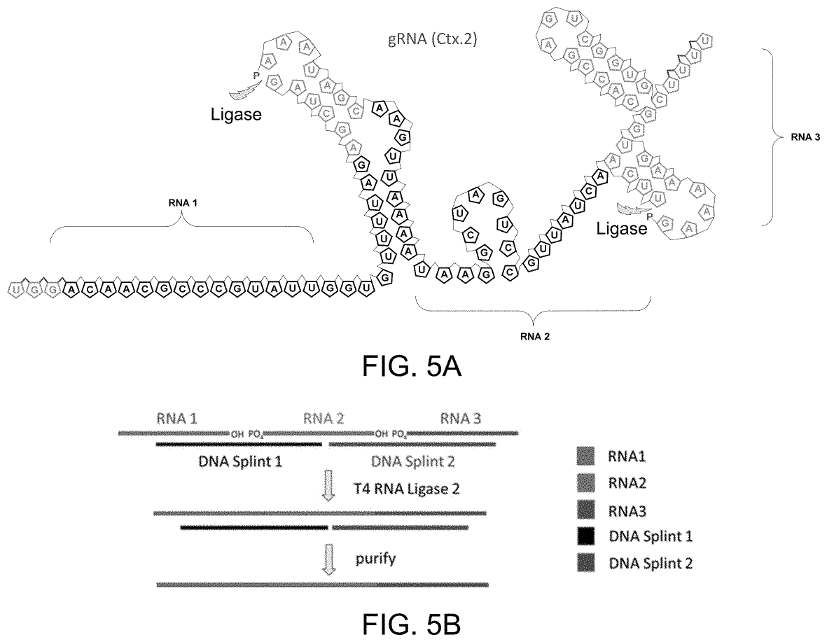

An exemplary gRNA molecule that targets Ctx.2 gene was synthesized using the splint-mediated ligation method described herein. The gRNA was split into three RNA fragments (RNA 1, RNA 2 and RNA 3) as shown in FIG. 5A, and the three RNA fragments were used together with two DNA splint oligonucleotides (DNA Splint 1 and DNA Splint 2) as shown in FIG. 5B to generate the gRNA.

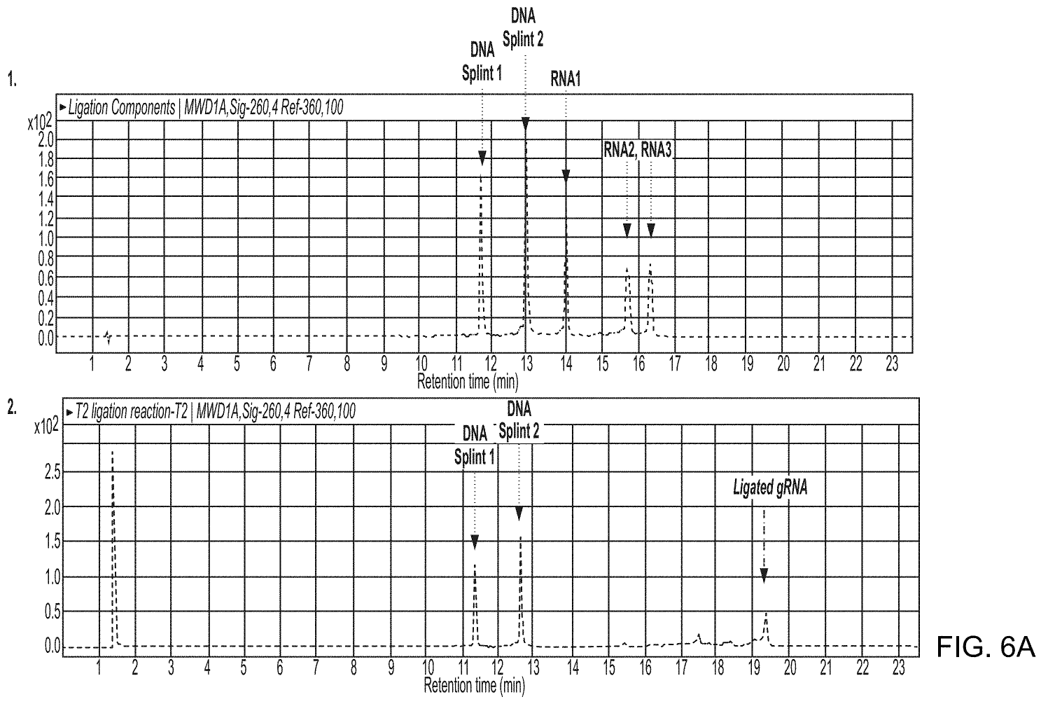

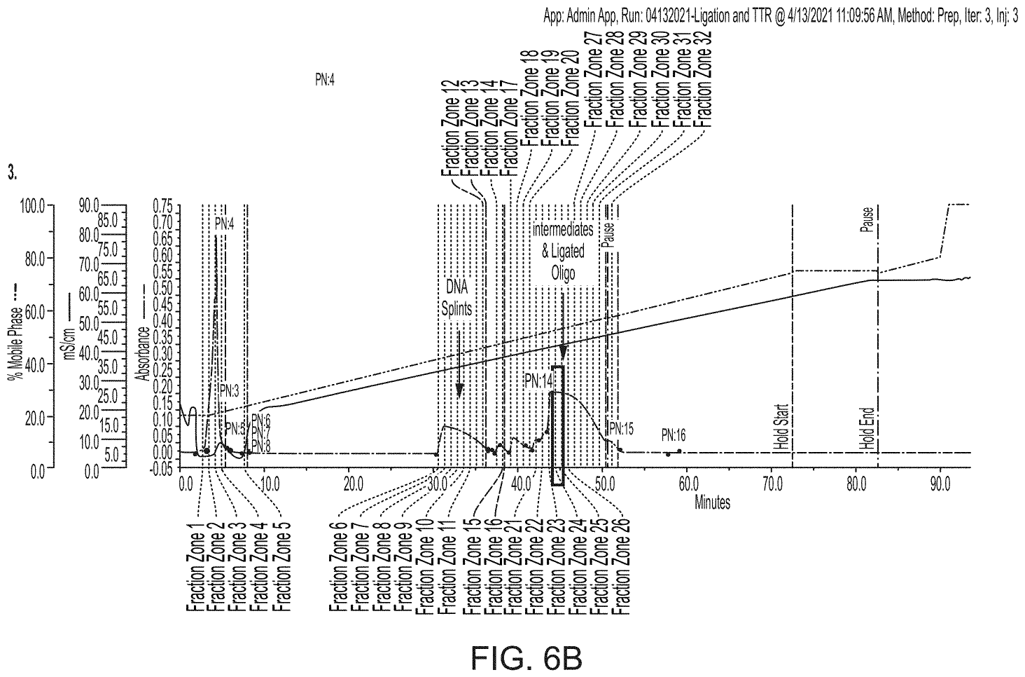

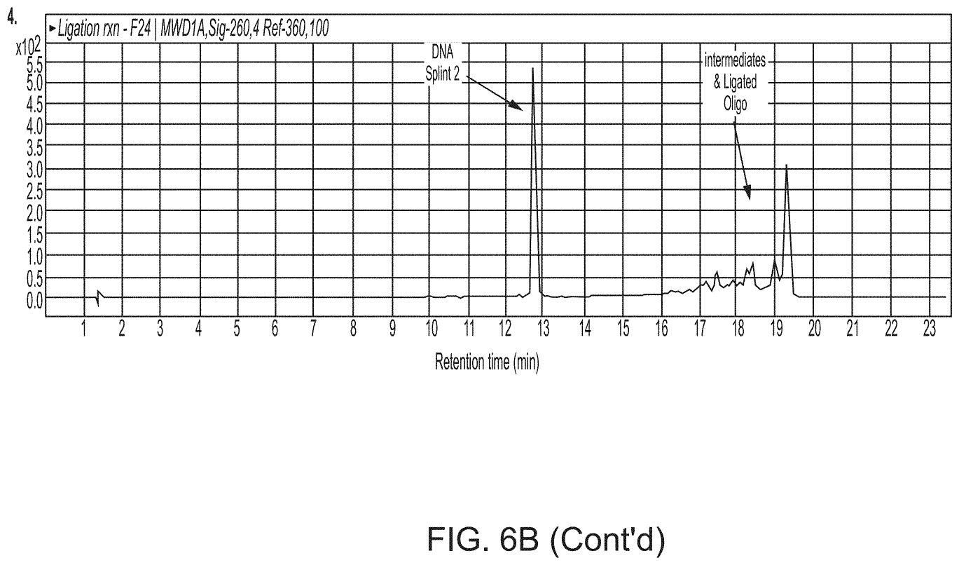

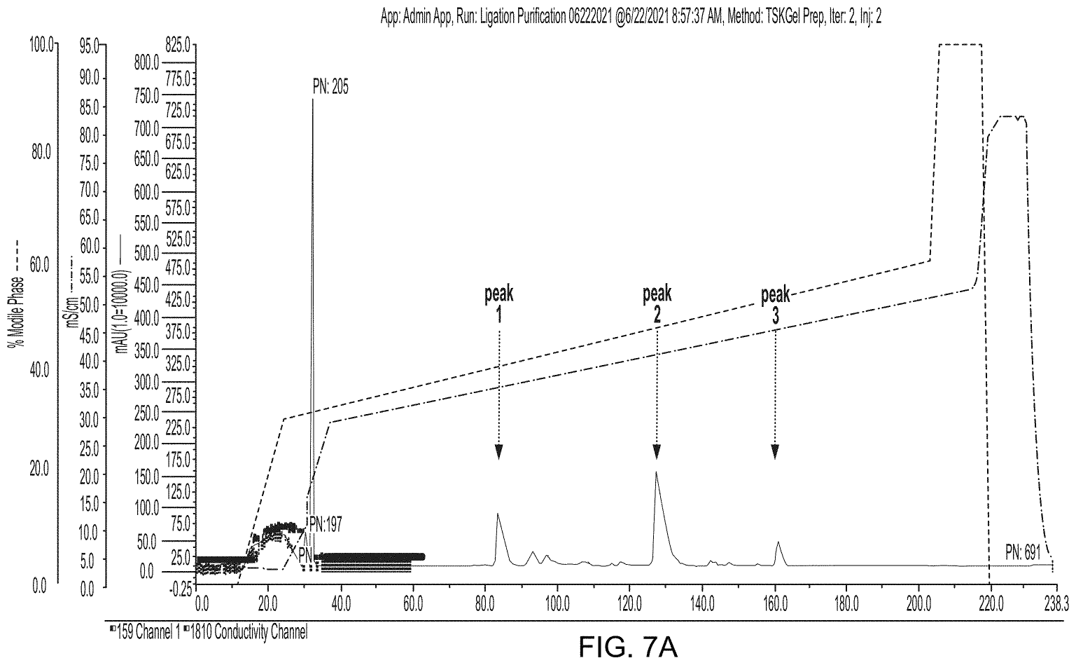

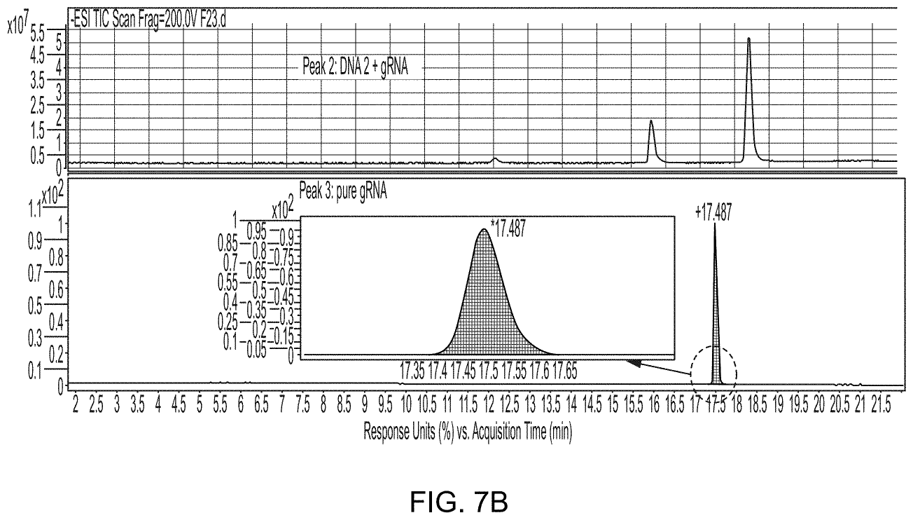

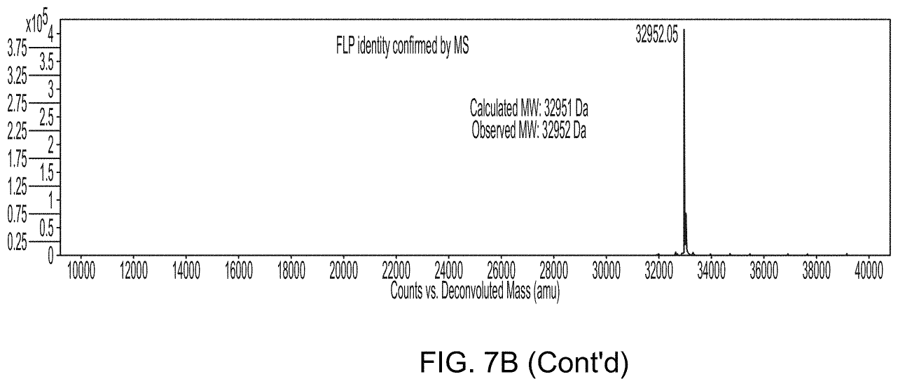

The pre-purified ligation components and the crude ligation reaction were analyzed by HPLC (FIG. 6A) which showed successful synthesis of ligated gRNA. Ligation reaction mixture was separated using HPLC, which lead to a surprising discovery that it is challenging to separate the ligated gRNA products from the DNA Splint oligonucleotides and short-mers (FIG. 6B). High-solution column was used to further purify the ligation reaction mixture, which indicated a significant challenge to separate a portion of the gRNA molecules from DNA Splint 2 (FIGS. 7A and 7B). Without being bound by any particular theory, it is believed that that portion of the gRNA molecules formed stable duplex with DNA Splint 2.

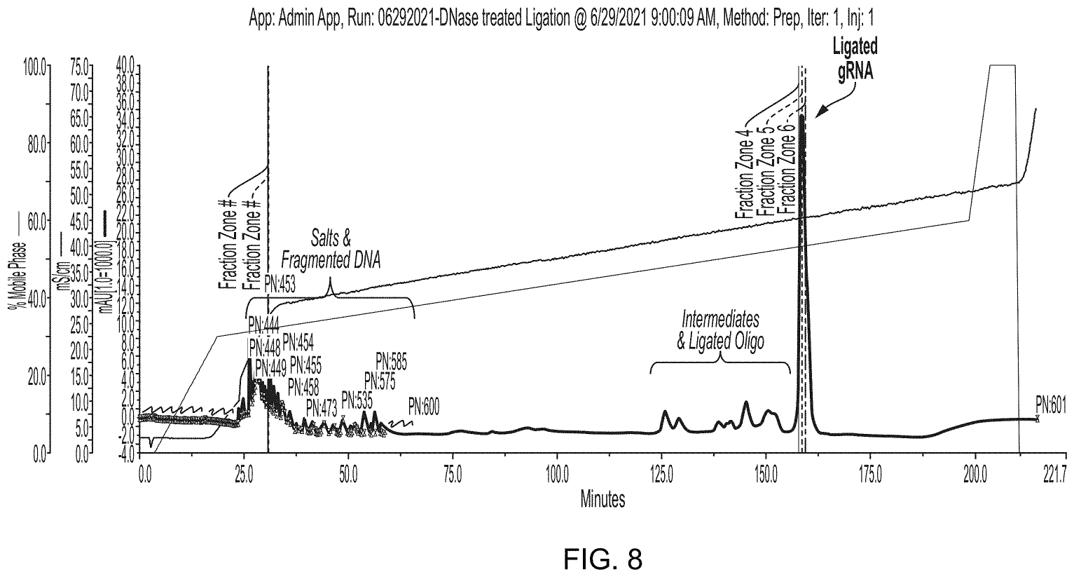

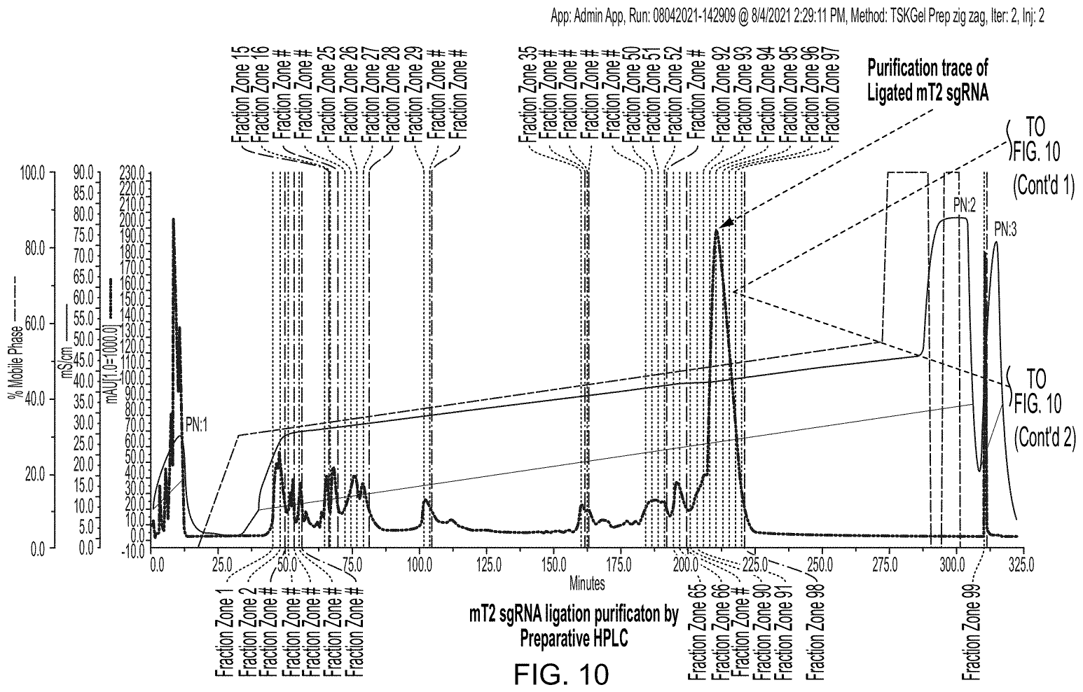

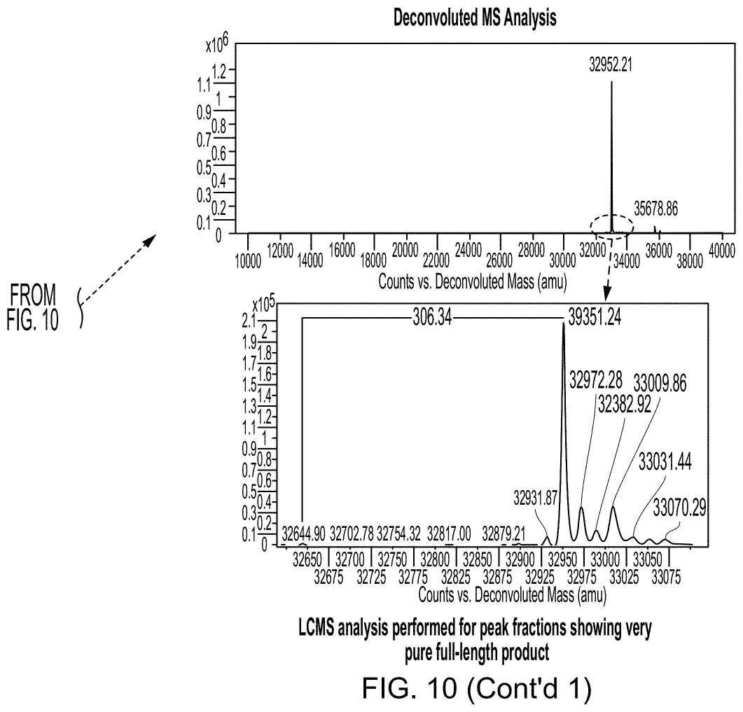

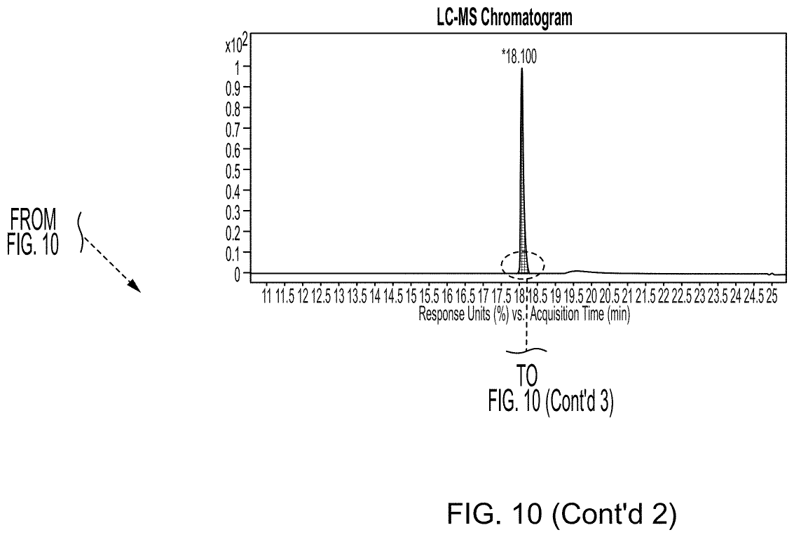

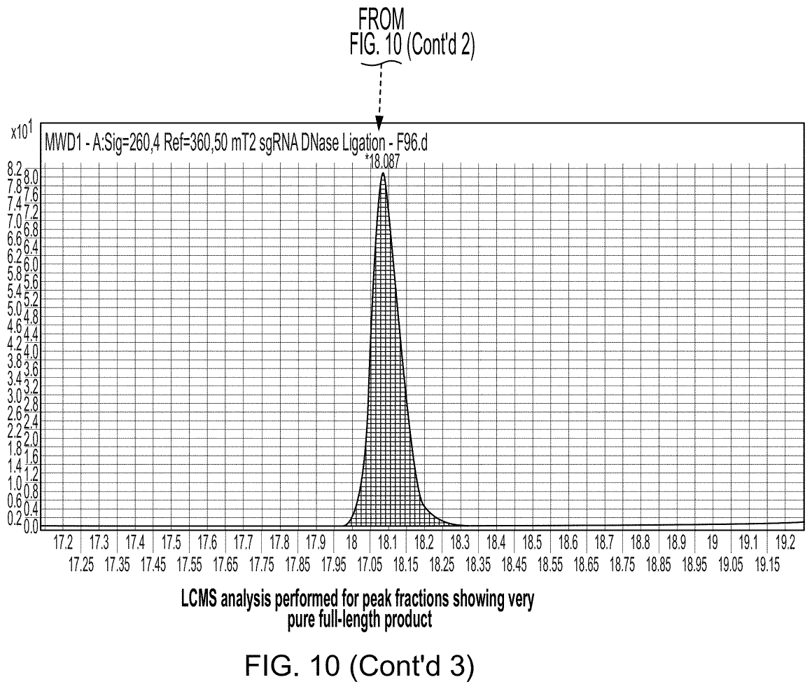

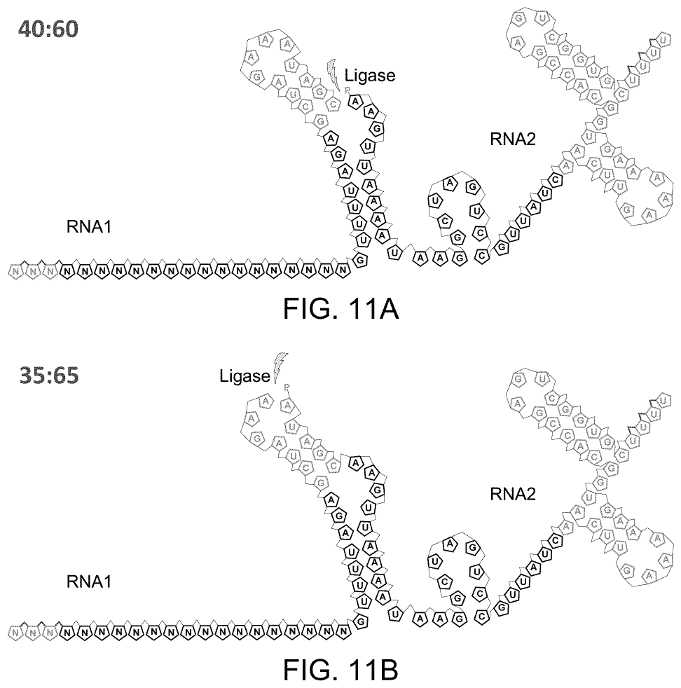

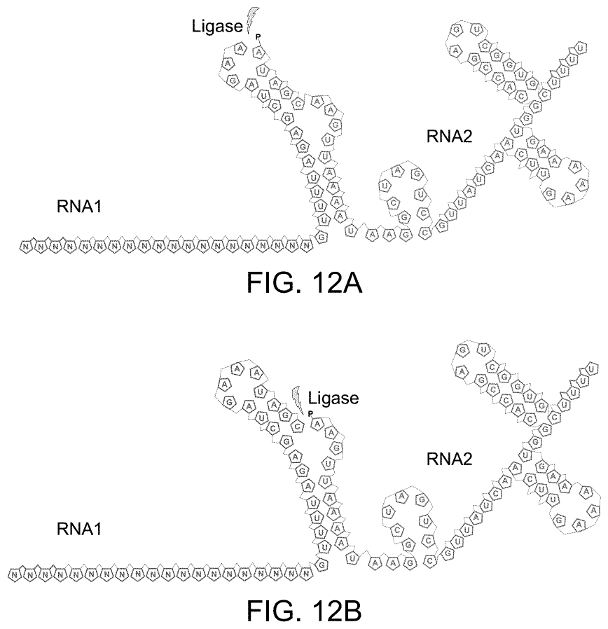

DNase treatment was carried out to digest the DNA Splint oligonucleotides after the ligation reaction. FIGS. 8-10 demonstrated that the DNase treatment significantly improved the yield of free gRNA. Without being bound by any particular theory, it is believed that that the DNase treatment digested DNA Splint 2 in the duplex formed by DNA Splint 2 and gRNA, and thus resulted in higher yield of gRNA and gRNA in higher purity.

### Example 2

### gRNA Synthesis by Splint-Mediated Two-Piece Ligation Approach

This example demonstrates gRNA synthesis by ligating two RNA fragments with one DNA splint oligonucleotide.

Example 1 demonstrates splint-mediated experiments by fragmenting guide RNA in three pieces. Success of this process primarily depends on purity of the first RNA fragment (RNA1). Since other fragments (e.g., RNA2, RNA3 etc.) will have a phosphate as an anchor to ligate, only FLP with synthetic phosphate will participate in ligation. Other synthetic impurities (N-1, N-2 . . . ) will not have any phosphate and will not participate in ligation. Without being bound by any particular theory, it is believed that by providing RNA1 in a short length and in very high purity while the other RNA fragment (RNA2) in a longer length and in a sufficient purity, it should be sufficient for producing highly pure gRNA.

The gRNA can be fragmented and modified in multiple ways (e.g., with 2′-O-methyl group and/or phosphorothioate linkage). For example, a first RNA (1stRNA) can be fragmented into two fragments: CUAACAGUUGCUUUUAUCACGUUUUAGAG CUAGAAAUAGCAAGUUAAAAU (SEQ ID NO: 2) and AAGGCUAGUCCGUUAU CAACUUGAAAAAGUGGCACCGAGUCGGUGCUUUU (SEQ ID NO: 3), or CUAACAGUUGCUUUUAUCACGUUUUAGAGCUAGAAAUAGC (SEQ ID NO: 4) and AAGUUAAAAUAAGGCUAGUCCGUUAUCAACUUGAAAAAGUGGCACC GAGUCGGUGCUUUU (SEQ ID NO: 5), or CUAACAGUUGCUUUUAUCAC GUUUUAGAGCUAGAA (SEQ ID NO: 6) and AUAGCAAGUUAAAAUAAGG CUAGUCCGUUAUCAACUUGAAAAAGUGGCACCGAGUCGGUGCUUUU (SEQ ID NO: 7). T2RNA can be fragmented into: GAGAACGCACCACUUUACGA GUUUUAGAGCUAGAAAUAGCAAGUUAAAAU (SEQ ID NO: 8) and AAGG CUAGUCCGUUAUCAACUUGAAAAAGUGGCACCGAGUCGGUGCUUUU (SEQ ID NO: 9), or GAGAACGCACCACUUUACGAGUUUUAGAGCUAGAAAUAGC (SEQ ID NO: 10) and AAGUUAAAAUAAGGCUAGUCCGUUAUCAACUU GAAAAAGUGGCACCGAGUCGGUGCUUUU (SEQ ID NO: 11), or GAGAA CGCACCACUUUACGAGUUUUAGAGCUAGAA (SEQ ID NO: 12) and AUAG CAAGUUAAAAUAAGGCUAGUCCGUUAUCAACUUGAAAAAGUGGCACCGAGUCGGUG CUUUU (SEQ ID NO: 13).

Table 1 provides the above RNA fragments with modifications and exemplary DNA splints used to ligate the RNA fragments.

TABLE 1
Exemplary RNA fragments and DNA splints

| RNA | | |
|---|---|---|
| fragment | Length | Sequence |
| 1stRNAIL | 50 nt | csusasACAGUUGCUUUUAUCACGUUUUAGAGCUA |
| | | GAAAUAGCAAGUUAAAAU |
| | | (SEQ ID NO: 38) |
| 1stRNA2L | 50 nt | PAAGGCUAGUCCGUUAUCAACUUGAAAAAGUGGCA |
| | | CCGAGUCGGUGCusususU |
| | | (SEQ ID NO: 39) |
| 1stRNAIL | 40 nt | csusasACAGUUGCUUUUAUCACGUUUUAGAGCUA |
| | | GAAAUAGC |
| | | (SEQ ID NO: 40) |
| 1stRNA2L | 60 nt | PAAGUUAAAAUAAGGCUAGUCCGUUAUCAACUUGA |
| | | AAAAGUGGCACCGAGUCGGUGCusususU |
| | | (SEQ ID NO: 41) |
| 1stRNAIL | 35 nt | csusasACAGUUGCUUUUAUCACGUUUUAGAGCUA |
| | | GAA |
| | | (SEQ ID NO: 42) |
| 1stRNA2L | 65 nt | PAUAGCAAGUUAAAAUAAGGCUAGUCCGUUAUCAA |
| | | CUUGAAAAAGUGGCACCGAGUCGGUGCusususU |
| | | (SEQ ID NO: 43) |
| T2RNA 1L | 50 nt | gsasgsAACGCACCACUUUACGAGUUUUAGAgcua |
| | | gaaauagcAAGUUAAAAU |
| | | (SEQ ID NO: 44) |
| T2RNA 2L | 50 nt | PAAGGCUAGUCCGUUAUCaacuugaaaaaguggca |
| | | ccgagucggugcusususu |
| | | (SEQ ID NO: 45) |
| T2RNA 1L | 40 nt | gsasgsAACGCACCACUUUACGAGUUUUAGAgcua |
| | | gaaauagc |
| | | (SEQ ID NO: 46) |
| T2RNA 2L | 60 nt | PAAGUUAAAAUAAGGCUAGUCCGUUAUCaacuuga |
| | | aaaaguggcaccgagucggugcusususu |
| | | (SEQ ID NO: 47) |
| T2RNA 1L | 35 nt | gsasgsAACGCACCACUUUACGAGUUUUAGAgcua |
| | | gaa |
| | | (SEQ ID NO: 48) |
| T2RNA 2L | 65 nt | PauagcAAGUUAAAAUAAGGCUAGUCCGUUAUCaa |
| | | cuugaaaaaguggcaccgagucggugcusususu |
| | | (SEQ ID NO: 49) |
| 35:65 | 33 nt | CTTATTTTAACTTGCTATTTCTAGCTCTAAAAC |
| Splint | | (SEQ ID NO: 14) |
| 35:65 | 36 nt | CCTTATTTTAACTTGCTATTTCTAGCTCTAAAACGT |
| Splint | | (SEQ ID NO: 15) |
| 40:60 | 32 nt | TAGCCTTATTTTAACTTGCTATTTCTAGCTCT |
| Splint | | (SEQ ID NO: 16) |
| 40:60 | 35 nt | CTAGCCTTATTTTAACTTGCTATTTCTAGCTCTAA |
| Splint | | (SEQ ID NO: 17) |
| 50:50 | 30 nt | CGGACTAGCCTTATTTTAACTTGCTATTTC |
| Splint | | (SEQ ID NO: 18) |
| 50:50 | 34 nt | AACGGACTAGCCTTATTTTAACTTGCTATTTCTA |
| Splint | | (SEQ ID NO: 19) |

A, G, U, C: ribonucleotide
a, g, u, c: 2′-O-methyl-nucleotide
s: Phosphorothioate linkage
P: Phosphate

In some embodiments, RNA fragments 1stRNA1L (40 nt) and 1stRNA2L (60 nt) together with DNA splint 40:60 of 32 nt (SEQ ID NO: 16) were dissolved in water to make a 1 mM stock solution. The DNA/RNA mixture (e.g., at equal molar ratio) in water was heated at 90° C. for 3 minutes, followed by slow cooling to 27° C. at machine specified rate. Ligase buffer (e.g., 10× buffer), ligase enzyme and RNase inhibitor were added to the mixture to form a ligation reaction mixture. The components in the ligation reaction mixture were provided in Table 2 below.

TABLE 2
Components of an exemplary ligation mixture

| Component | Volume | |
|---|---|---|
| H₂O | 32 | mL |
| 10× Reaction Buffer | 4 | mL |
| DNA splint & RNA fragments (1 mM) | 400 | μL × 5 |
| T4 RNA Ligase 2 | 1200 | μL (300 U) |
| RNase Inhibitor | 800 | uL |
| Total | 40 | mL |

The ligation reaction mixture was then incubated for 6 hours at 37° C. The ligation reaction can be terminated by quenching with EDTA and then analyzed using IE-HPLC for every 30 min. The reaction mixture was passed through spin column (10 KD cut off) and taken for purification by Prep-IEX-HPLC. The final amounts of the synthesized first gRNA (also referred to as “1stgRNA” or “1stRNA” herein) using the two-piece ligation approach are shown in Table 3 below in comparison to the same gRNA product synthesized using a three-piece ligation approach.

TABLE 3
Final amounts of synthesized gRNA

| Sample | mg | OD |
|---|---|---|
| 1stRNA 35-65 | 2.0 | 49 |
| 1stRNA 40-60 | 1.3 | 33 |
| 1stRNA, 3 piece | 0.7 | 16 |

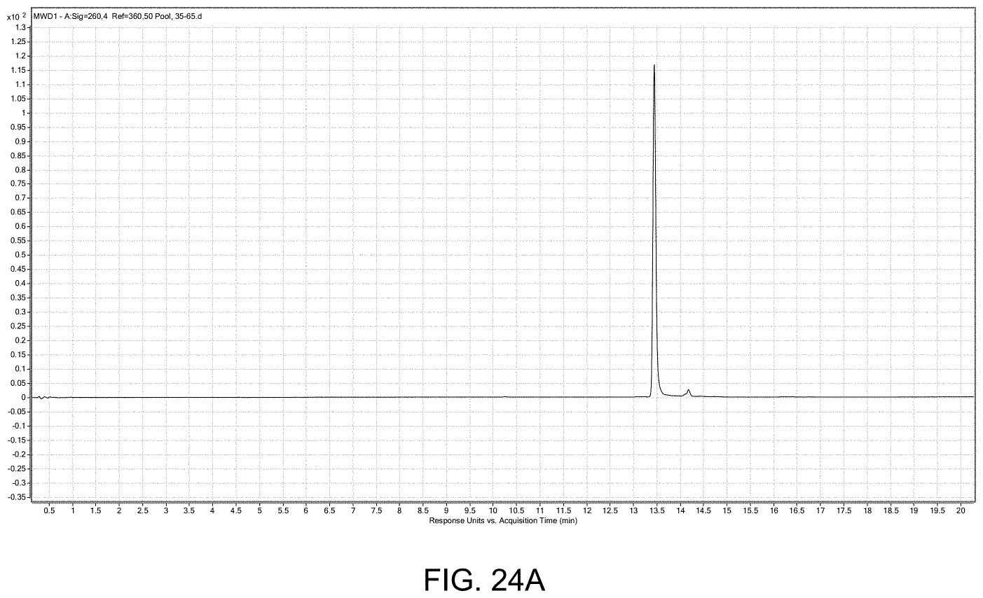

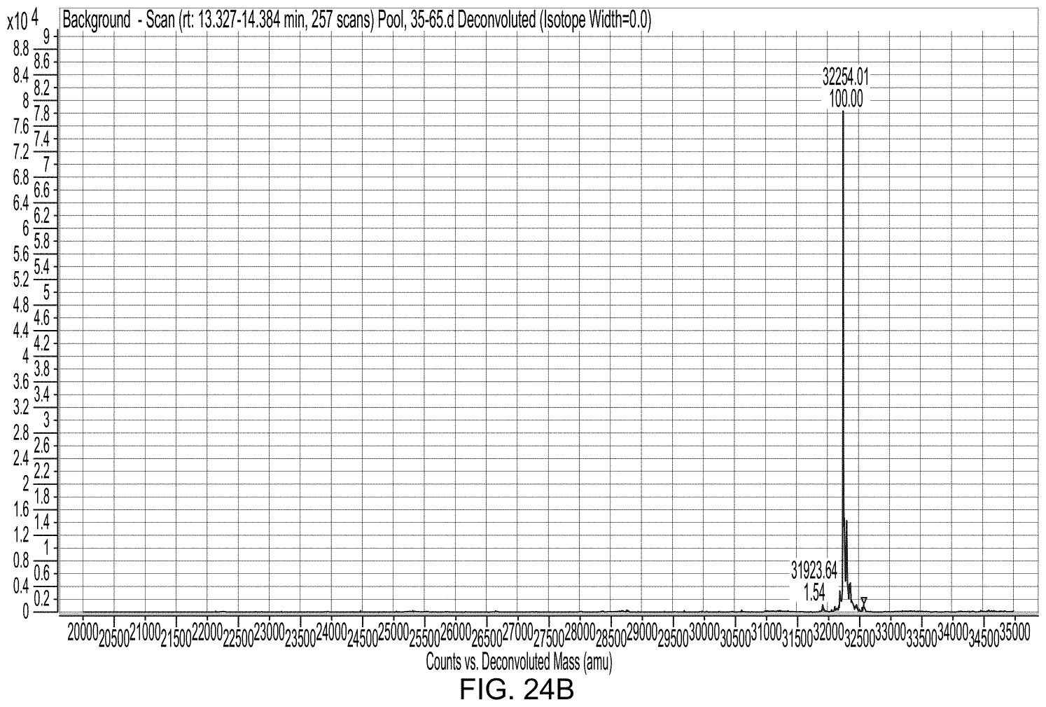

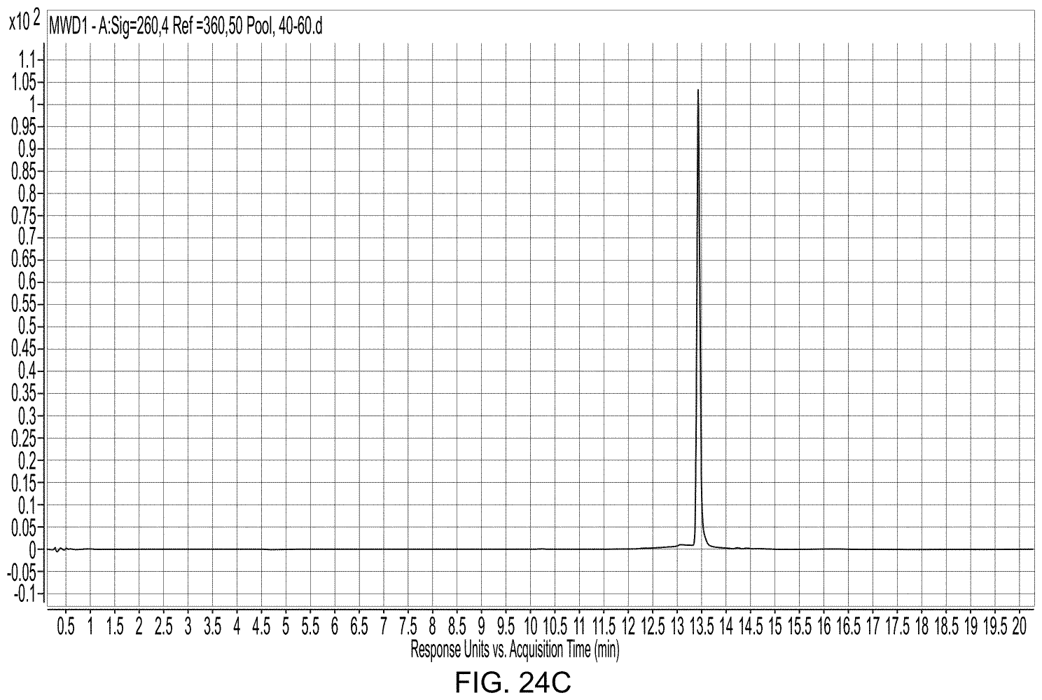

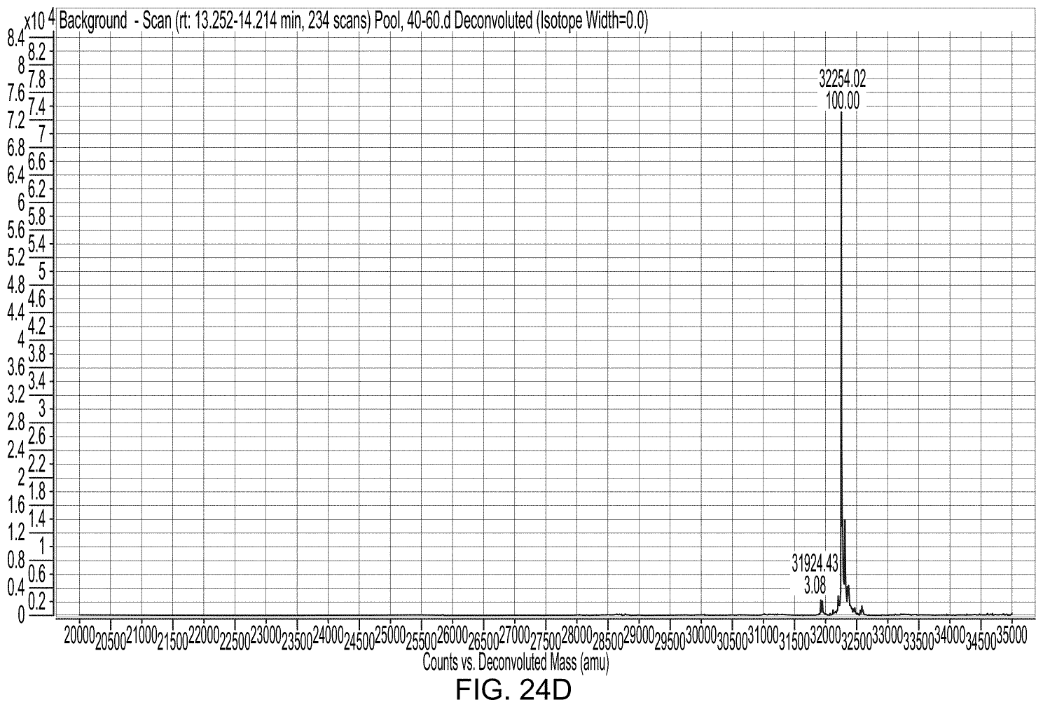

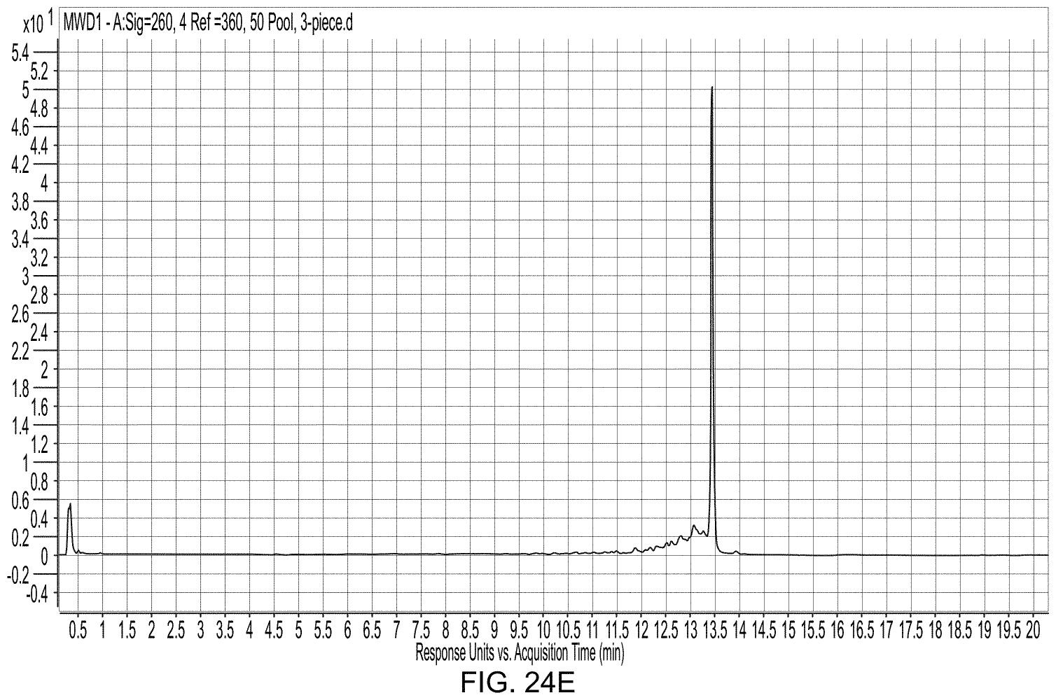

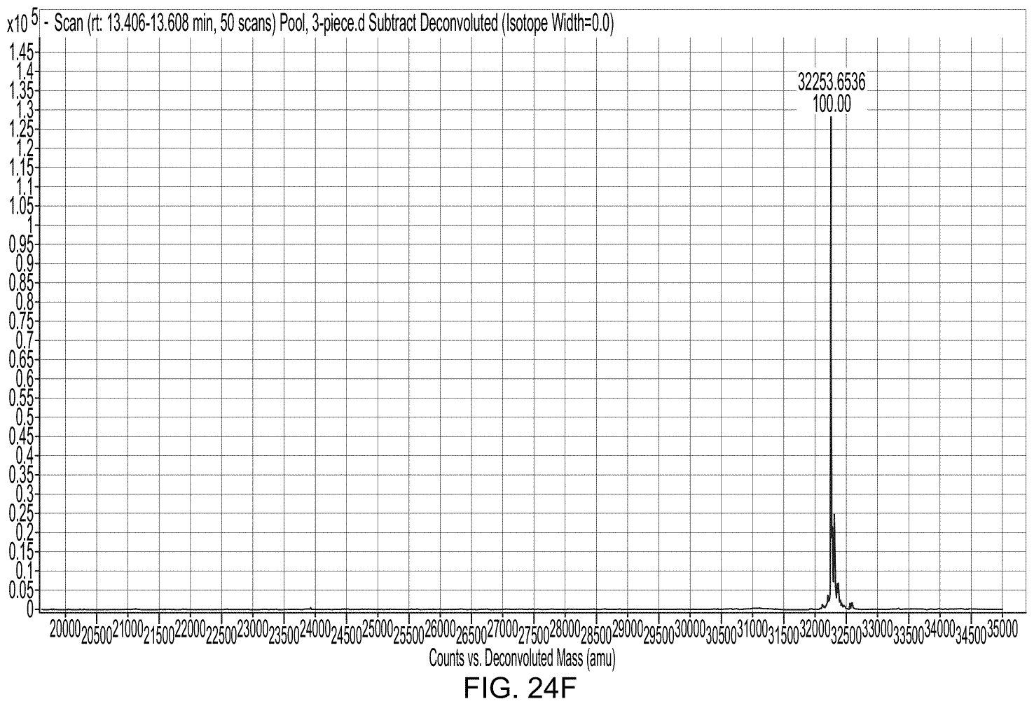

FIGS. 24A-F show the UPLC trace and HPLC/MS chromatogram of the first gRNA synthesized using a 35-65 two-RNA fragment design (FIGS. 24A-B), a 40-60 two-RNA fragment design (FIGS. 24C-D), and the three-RNA fragment design (FIGS. 24E-F). Table 4 summarize the peak amu and purity/impurity data from the UPLC and MS analysis of the synthesized first gRNA.

TABLE 4
UPLC and MS analysis of the gRNA

| Sample | Peak Amu | Impurity | Mass-based purity |
|---|---|---|---|
| 1stRNA 35-65 | 32254 | N +− 1 | 97.1% |
| 1stRNA 40-60 | 32254 | N +− 1 | 92.6% |
| 1stRNA 3-piece | 32253 | N + 1 | 96.1% |

### Example 3

### Ligation Reaction in Large Scale without DNase Treatment

This example demonstrates a ligation reaction and purification in large scale without DNase treatment.

RNA fragments (e.g., RNA1, RNA2) and DNA splints (e.g., DNA splint1) were dissolved in water to make a 1 mM stock solution. To prepare nicked DNA/RNA hybrid substrates, the DNA splints and RNA fragments were mixed (e.g., at equal molar ratio) to make a DNA/RNA mixture. The DNA/RNA mixture was heated at 90° C. for 3 minutes in an Eppendorf tube, followed by slow cooling to 37° C. at machine specified rate. Ligase buffer and ligase enzyme were added to the mixture to form a ligation reaction mixture. The components in the ligation reaction mixture were provided in Table 5 below. The ligation reaction mixture was then incubated for 6 hours at 37° C. The ligation reaction can be terminated by quenching with EDTA and then analyzed using IE-HPLC.

TABLE 5
Components of an exemplary ligation mixture

| Component | Volume | |
|---|---|---|
| H₂O | 7900 | μL |
| 10× Reaction Buffer | 1000 | μL |
| DNA splint & RNA fragments (1 mM) | 100 | μL × 5 |
| T4 RNA Ligase 2 | 500 | μL (300 U) |
| RNase Inhibitor | 100 | uL |
| Total | 10000 | μL |

The pre-purified ligation components and the crude ligation reaction were analyzed by HPLC (FIG. 6A). FIG. 6A shows ligation purification by IE HPLC: panel 1 shows pre-purification ligation components (RNA1 fragment, RNA2 fragment, RNA3 fragment, DNA Splint 1, and DNA Splint 2), panel 2 shows components of crude ligation reaction (DNA Splint 1, DNA Splint 2, and ligated gRNA reaction product). Formation of ligated gRNA peak can be clearly seen from the HPLC profile.

Next, the crude ligation reaction was diluted 2× with buffer A and loaded on 250 mm×9 mm DNAPac IE column. Buffer A contains 20 mM sodium phosphate and 10% acetonitrile having a pH 8.5. Table 6 below shows an exemplary IE gradient elution. Buffer B contains 20 mM sodium phosphate, 10% acetonitrile, and 1M sodium bromide having a pH 8.5.

TABLE 6
IE gradient

| CV | % Buffer B |
|---|---|
| 1 | 0 |
| 2 | 0-25 |
| 15 | 25-60 |
| 1 | 100 |
| 3 | 0 |

FIG. 6B, panel 3 shows HPLC separation of DNA Splints and Intermediates & Ligated Oligo, and panel 4 shows post-purified analysis of the components shown in the blue box of panel 3 (intermediates & ligated oligos). Fractions 6-13 of panel 3 contained mixture of DNA splints 1 & 2. Fractions 23-32 of panel 3 showed FLP mixed with ligated oligonucleotides and reaction intermediates. The results suggest that in some instances such as when the DNA splints are in a longer length, optimization for product purification may be necessary to further separate the gRNA molecules from DNA Splint oligonucleotides and short-mers.

### Example 4

### Ligation Reaction in Large Scale with DNase Treatment

This example demonstrates a ligation reaction and purification in large scale with DNase treatment.

In some examples, it may be challenging to purify full length gRNA from DNA splints due to the tight binding of DNA splints with target RNA. It has been shown that in some examples DNA Splint 1 can have a Tm of ˜54° C. and DNA Splint 2 can have a Tm of ˜62° C. Tm will also increase dramatically with increasing salt concentration. In this example, DNase treatment was carried out to digest the DNA splint oligonucleotides after the ligation reaction.

The mT2RNA were fragmented and modified (e.g., with 2′-O-methyl group and/or phosphorothioate linkage). For example, mT2 RNA can be fragmented into three RNA fragments: GAGAACGCACCACUUUACGAGUUUUAGAGCUAG (SEQ ID NO: 20), AAAUAGCAAGUUAAAAUAAGGCUAGUCCGUUAUCAACUU (SEQ ID NO: 21), and GAAAAAGUGGCACCGAGUCGGUGCUUUU (SEQ ID NO: 22). Table 7 shows exemplary modifications of the above RNA fragments and DNA splints used in this example.

TABLE 7
Sequences of mT2 RNA fragments and DNA splints

| Oligos | # NTs | Sequence |
|---|---|---|
| mT2 RNA 1 | 33 | mG*mA*mG*rArArCrGrCrArCrCrArCrUrUr |
| | | UrArCrGrArGrUrUrUrUrArGrAmGmCmUmAm |
| | | G (SEQ ID NO: 50) |
| RNA 2 | 39 | PmAmAmAmUmAmGmCrArArGrUrUrArArArAr |
| | | UrArArGrGrCrUrArGrUrCrCrGrUrUrArUr |
| | | CrAmAmCmUmU (SEQ ID NO: 51) |
| RNA 3 | 28 | PmGmAmAmAmAmAmGmUmGmGmCmAmCmCmGmAm |
| | | GmUmCmGmGmUmGmCmU*mU*mU*mU |
| | | (SEQ ID NO: 52) |
| Splint | 36 | CCTTATTTTAACTTGCTATTTCTAGCTCTAAAAC |
| 1→2 | | TC (SEQ ID NO: 23) |
| Splint 2 3 | 48 | AAAAGCACCGACTCGGTGCCACTTTTTCAAGTTG |
| 2→3 | | ATAACGGACTAGCC (SEQ ID NO: 24) |

mA, mC, mG, mU: 2′-O-methyl-nucleotide
rA: riboadenosine; rG: riboguanosine; rC: ribocytosine;
rU: ribouridine P: Phosphate;
“*” = phosphorothioate linkage

mT2 RNA fragments (e.g., RNA1, RNA2, RNA3) and DNA splints (e.g., DNA splint1 and DNA splint2) were dissolved in water to make a 1 mM stock solution. To prepare DNA/RNA hybrid substrates, the DNA splints and RNA fragments were mixed (e.g., at equal molar ratio) to make a DNA/RNA mixture. The DNA/RNA mixture was heated at 90° C. for 3 minutes in an Eppendorf tube, followed by slow cooling to 37° C. at machine specified rate. Ligase buffer and ligase enzyme were added to the mixture to form a ligation reaction mixture. The components in the ligation reaction mixture were provided in Table 8 below. The ligation reaction mixture was then incubated for 6 hours at 37° C. When HPLC analysis has shown completion of reaction, 300 uL (600 U) Turbo DNase was added to the ligation reaction mixture and incubated for 1 hour. The reaction can be terminated by quenching with EDTA and then analyzed using IE-HPLC.

TABLE 8
Components of an exemplary ligation mixture

| Component | Volume | |
|---|---|---|
| H₂O | 810 | μL |
| 10× Reaction Buffer | 100 | μL |
| DNA splint & RNA fragments (1 mM) | 10 | μL × 5 |
| T4 RNA Ligase 2 | 30 | μL (300 U) |
| RNase Inhibitor | 10 | uL |
| Total | 1000 | μL |

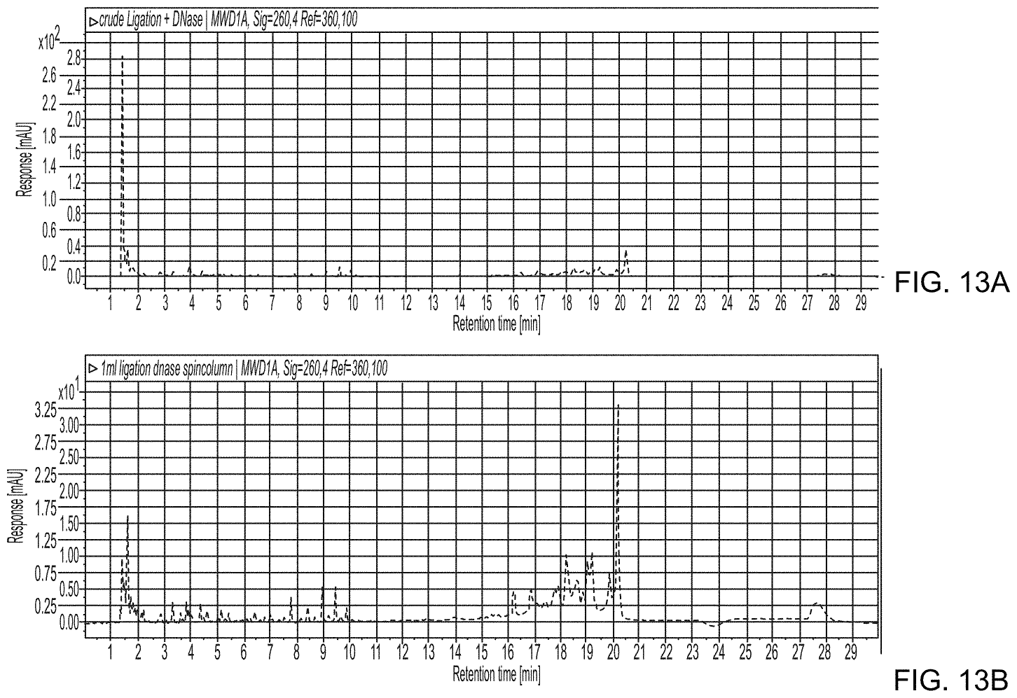

FIG. 13A-FIG. 13B show ligation purification by IE HPLC. FIG. 13A shows HPLC profile with DNase treatment. FIG. 13B shows HPLC profile after spin column purification. The final products were filtered through 10 kDa cutoff Amicon ultra column and the retentate was collected. The retentate was then diluted 10× with buffer A and loaded on 150 mm×7.6 mm TOSOH strong IE column. The same buffer and IE gradient used in Example 3 was used in this example.

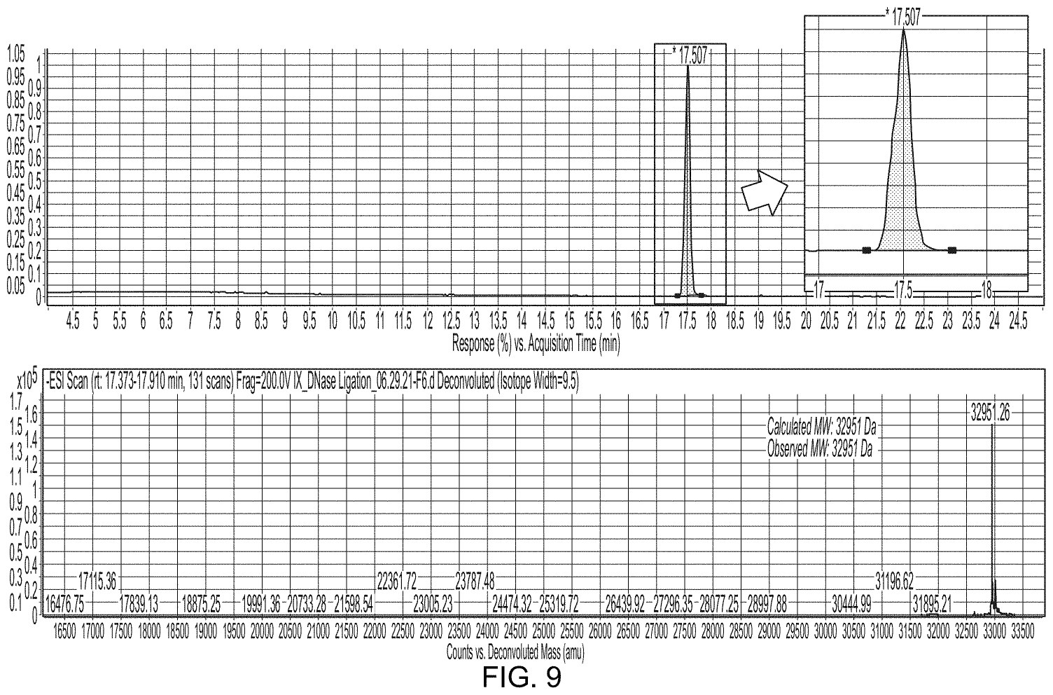

FIG. 8 shows HPLC separation of ligated gRNA from fragmented DNA and other intermediates and ligated Oligos. Fractions 4-6 in FIG. 8 showed pure FLP without any DNA splint or reaction intermediate contamination. FIG. 9 shows LC/MS data of purified gRNA.

The results demonstrated that the DNase treatment can significantly improve the purity and yield of ligated free gRNA.

### Example 5

### gRNA Synthesis by Short Splint- and Very Short Splint-Mediated Ligation

This example demonstrates that full-length gRNA molecules can be generated efficiently and in high purity using short splint- or very short splint-mediated ligation in the absence of DNase treatment.

By reducing the length of DNA splints, one can reduce Tm of the heteroduplex which would be beneficial for purification. The goal is to have Tm of each overlapping region of the heteroduplex to be greater than incubation temperature as it forms a stable complex while the Tm of overall DNA/gRNA duplex during purification should be less than 60° C. (or even lower) for ease of purification.

In one set of experiments, mT2RNA were fragmented and modified (e.g., with 2′-O-methyl group and/or phosphorothioate linkage). For example, mT2 RNA can be fragmented into three RNA fragments: GAGAACGCACCACUUUACGAGUUUUAGAGCUAG (SEQ ID NO: 25), AAAUAGCAAGUUAAAAUAAGGCUAGUCCGUUAUCAACUU (SEQ ID NO: 26), and GAAAAAGUGGCACCGAGUCGGUGCUUUU (SEQ ID NO: 27).

Table 9 shows the above mT2 RNA fragments with modifications and the DNA splints used in this set of experiments.

TABLE 9
Sequences of exemplary mT2 RNA
fragments and DNA splints

| | # | |
|---|---|---|
| Oligos | NTs | Sequence |
| mT2 RNA 1 | 33 | mG*mA*mG*rArArCrGrCrArCrCrArCrUrUrU |
| | | rArCrGrArGrUrUrUrUrArGrAmGmCmUmAmG |
| | | (SEQ ID NO: 53) |
| RNA 2 | 39 | PmAmAmAmUmAmGmCrArArGrUrUrArArArArU |
| | | rArArGrGrCrUrArGrUrCrCrGrUrUrArUrCr |
| | | AmAmCmUmU |
| | | (SEQ ID NO: 54) |
| RNA 3 | 28 | PmGmAmAmAmAmAmGmUmGmGmCmAmCmCmGmAm |
| | | GmUmCmGmGmUmGmCmU*mU*mU*mU |
| | | (SEQ ID NO: 55) |
| Splint 1 | 32 | TATTTTAACTTGCTATTTCTAGCTCTAAAACT |
| short | | (SEQ ID NO: 28) |
| Splint 2 | 27 | GTGCCACTTTTTCAAGTTGATAACGGA |
| short | | (SEQ ID NO: 29) |
| Splint 1 | 26 | TTTTAACTTGCTATTTCTAGCTCTAA |
| very short | | (SEQ ID NO: 30) |
| Splint 2 | 23 | GCCACTTTTTCAAGTTGATAACG |
| very short | | (SEQ ID NO: 31) |

mA, mC, mG, mU: 2′-O-methyl-nucleotide rA: riboadenosine;
rG: riboguanosine; rC: ribocytosine; rU: ribouridine
P: Phosphate = phosphorothioate

mT2 RNA fragments (e.g., mT2 RNA1, RNA2 and RNA3 of Table 9) and DNA splints (e.g., Splint 1 short and Splint 2 short or Splint 1 very short and Splint 2 very short of Table 9) were dissolved in water to make a 1 mM stock solution. To prepare DNA/RNA hybrid substrates, the DNA splints and RNA fragments were mixed (e.g., at equal molar ratio) to make a DNA/RNA mixture. The DNA/RNA mixture was then heated at 90° C. for 3 minutes in an Eppendorf tube, followed by slow cooling to a desired temperature at machine specified rate. Ligase buffer and ligase enzyme were added to the mixture to form a ligation reaction mixture. The components in the ligation reaction mixture were provided in Table 10 below. To validate the ligation reaction will take place at lower temperature, three reactions were carried out at 33° C., 30° C. and 27° C. for 6 h according to the procedure described in Example 3. All were carried out at 100 μL scale.

TABLE 10
Components of an exemplary ligation mixture

| Component | Volume | |
|---|---|---|
| H₂O | 81 | μL |
| 10× Reaction Buffer (NEB #B0239S) | 10 | μL |
| DNA splint & RNA fragments (1 mM) | 1 | μL × 5 |
| T4 RNA Ligase 2 | 3 | μL |
| RNase inhibitor | 1 | μL |
| Total | 100 | μL |

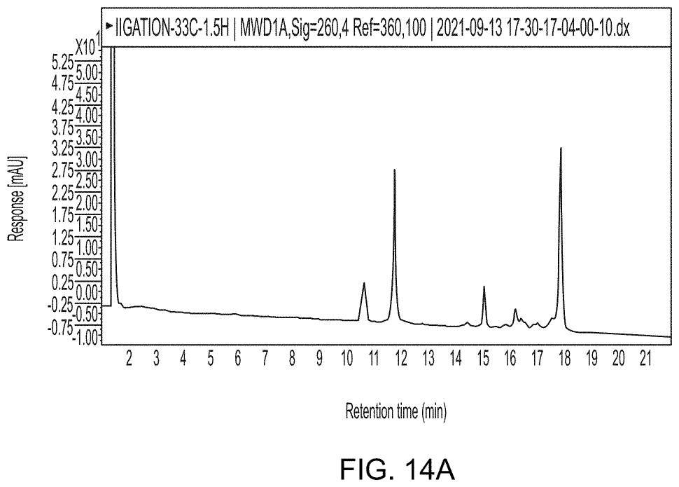

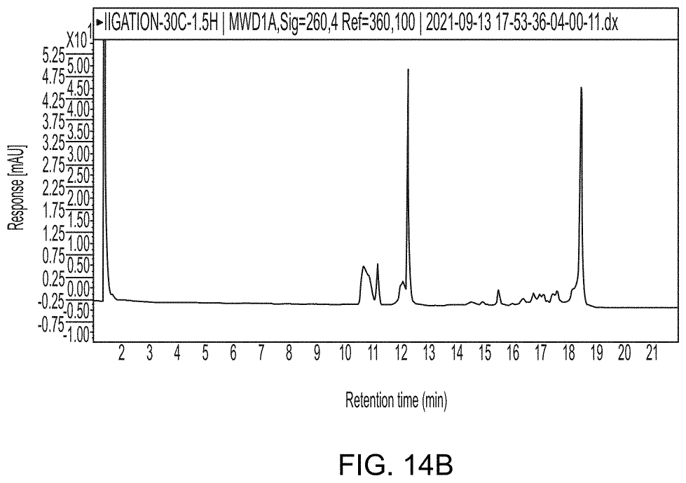

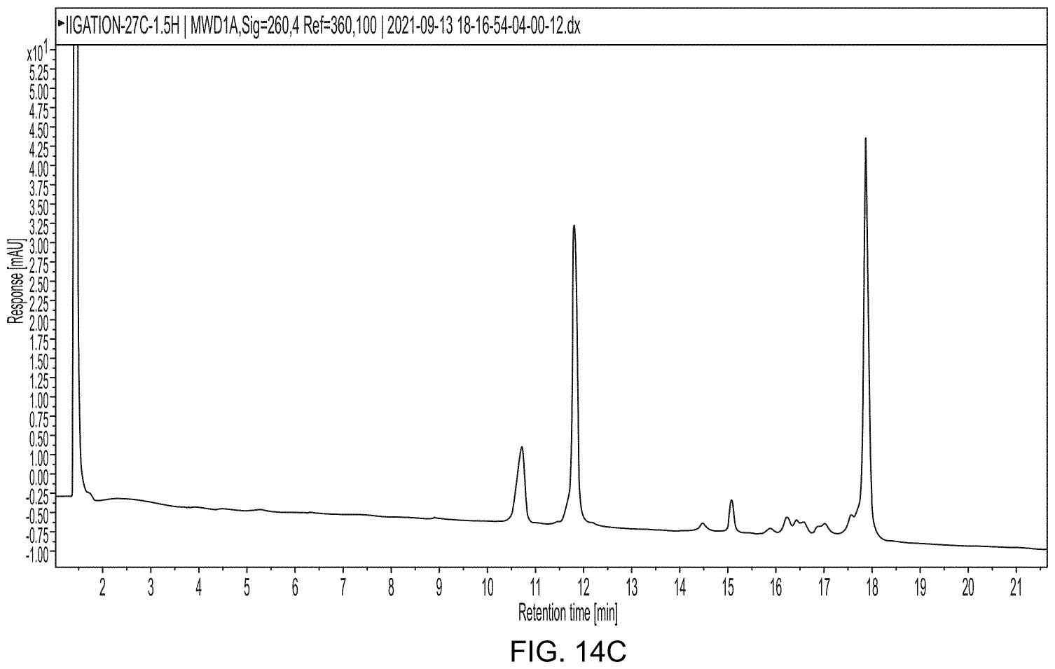

The pre-purified ligation components and the crude ligation reaction were analyzed by HPLC (FIGS. 14A-C). FIGS. 14A-C show ligation purification by IE HPLC carried out at 33° C., 30° C. and 27° C., respectively. The results showed that short and very short DNA splints can mediate a successful ligation reaction at a low temperature (e.g., 27° C.).

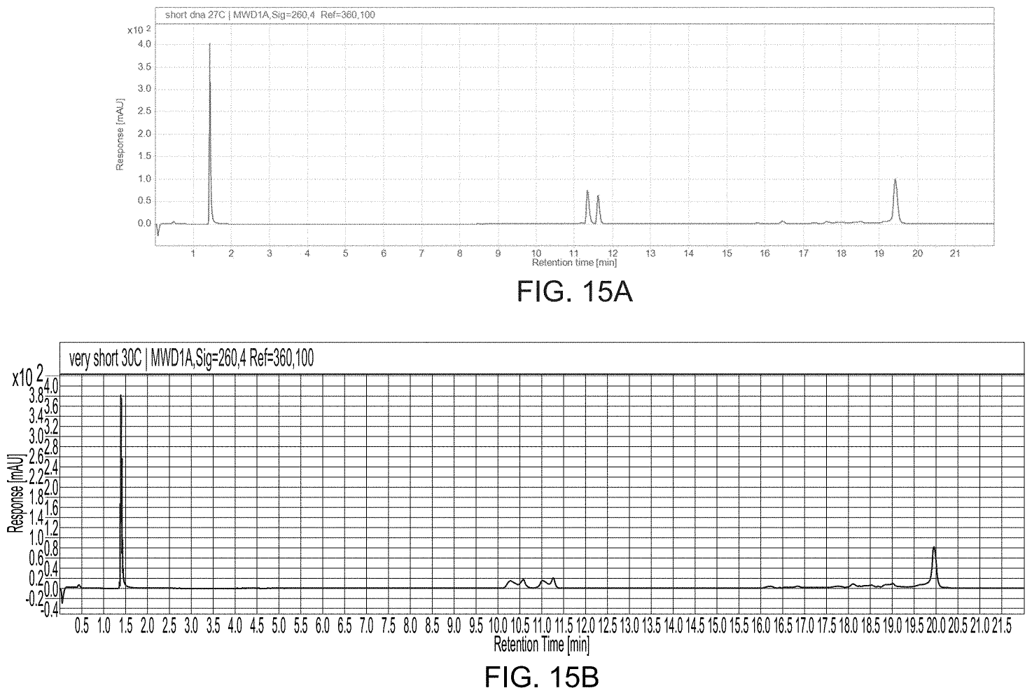

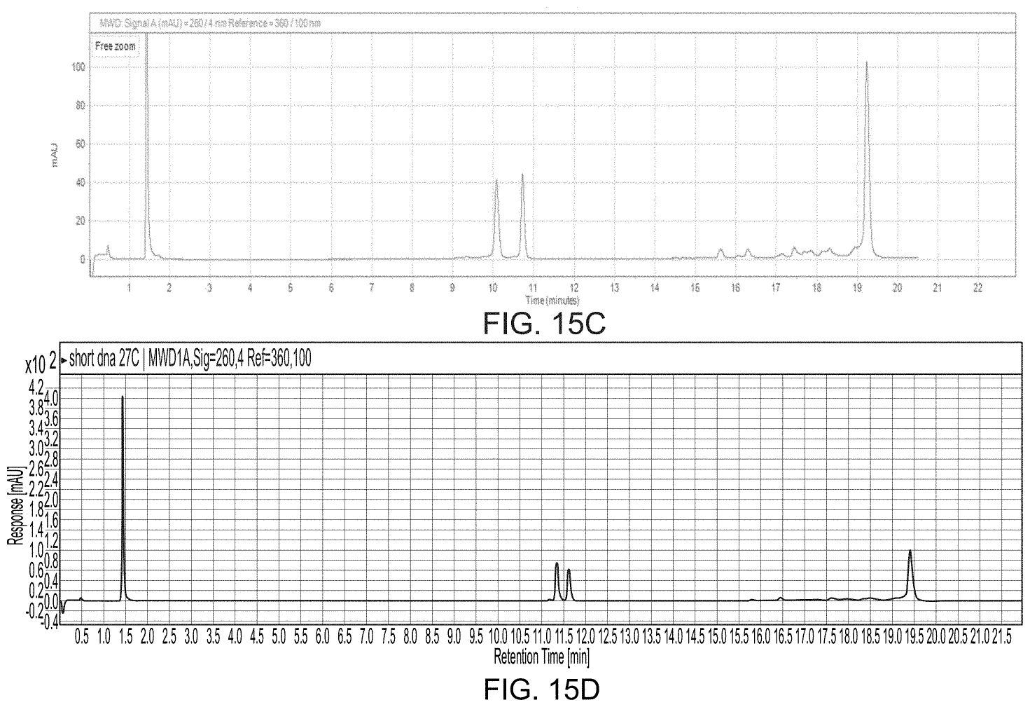

The ligation reaction was further evaluated using short splints and very short splints. Four sets of reactions were carried out: short splint mediated ligation at 27° C. and 30° C. and very short splint mediated ligation at 27° C. and 30° C. The crude ligation reactions were analyzed by HPLC (FIGS. 15A-D). FIGS. 15A-B show ligation purification by IE HPLC carried out with short DNA splints at 27° C. and 30° C., respectively. FIGS. 15C-D show ligation purification by IE HPLC carried out with very short DNA splints at 27° C. and 30° C., respectively. The results showed that both short and very short DNA splints can mediate a successful ligation reaction at both temperatures.

Next, large scale (2 ml) ligation reaction and purification were carried out using very short splints according to the procedure previously described in Example 3. The components in the ligation reaction mixture were provides in Table 11 below. The ligation reaction mixture was incubated for 6 hours at 27° C. The ligation reaction can be terminated by quenching with EDTA and then purified and analyzed using IE-HPLC.

TABLE 11
Components of an exemplary ligation mixture

| Component | Volume | |
|---|---|---|
| H₂O | 1630 | μL |
| 10× Reaction Buffer | 200 | μL |
| DNA splint & RNA fragments (1 mM) | 20 | μL × 5 |
| T4 RNA Ligase 2 | 60 | μL (300 U) |
| RNase Inhibitor | 10 | uL |
| Total | 2000 | μL |

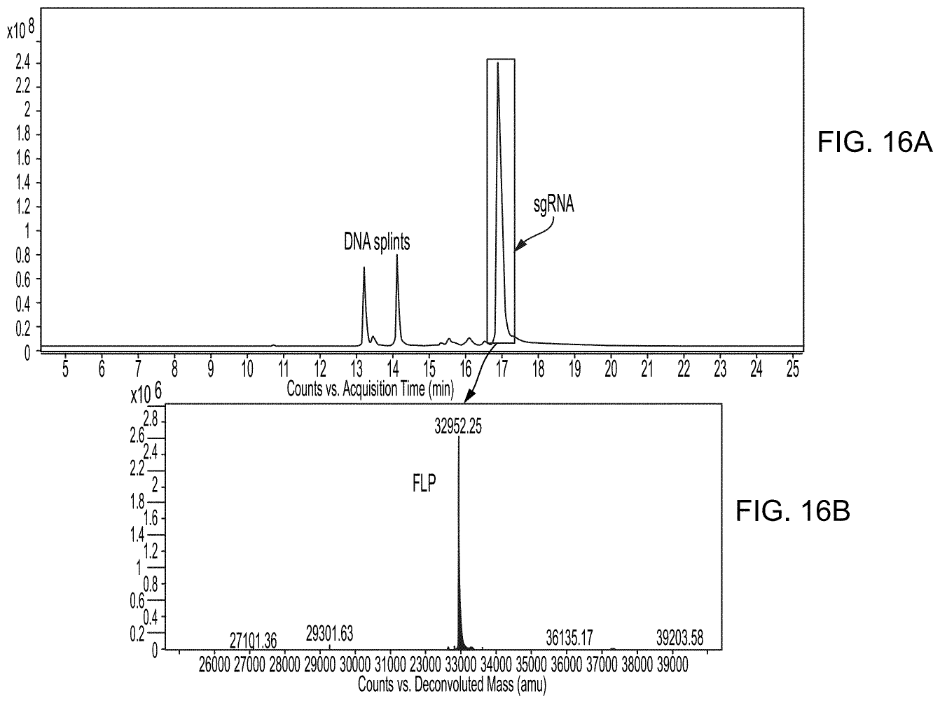

FIGS. 16A-B show LC/MC data of purified reaction products. FIG. 16A shows LC/MC data of the large-scale reaction. FIG. 16B shows deconvoluted MS analysis of full-length product. The results demonstrated that very short splints mediated ligation can result in improvement in the purity and yield of ligated free gRNA.

To further purify the ligation reaction mixture using preparative IE-HPLC, the crude ligation reaction was diluted 10× with buffer A and loaded on 150 mm×7.6 mm TOSOH strong IE column. Table 12 shows the IE gradient elution used in this example.

TABLE 12
IE gradient

| CV | % B |
|---|---|
| 1 | 0 |
| 2 | 0-30 |
| 15 | 30-55 |
| 1 | 100 |
| 3 | 0 |

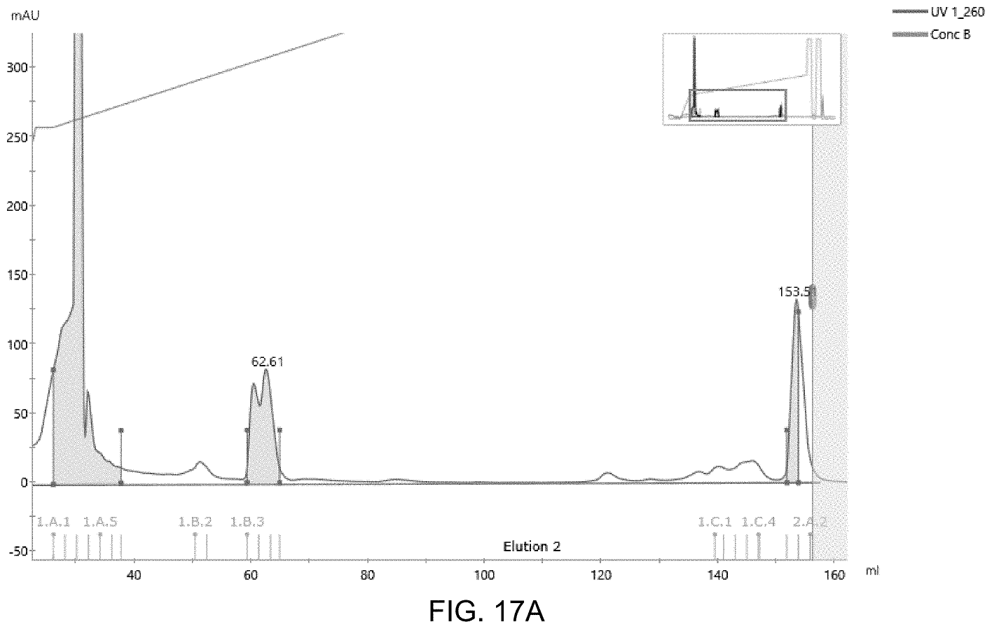

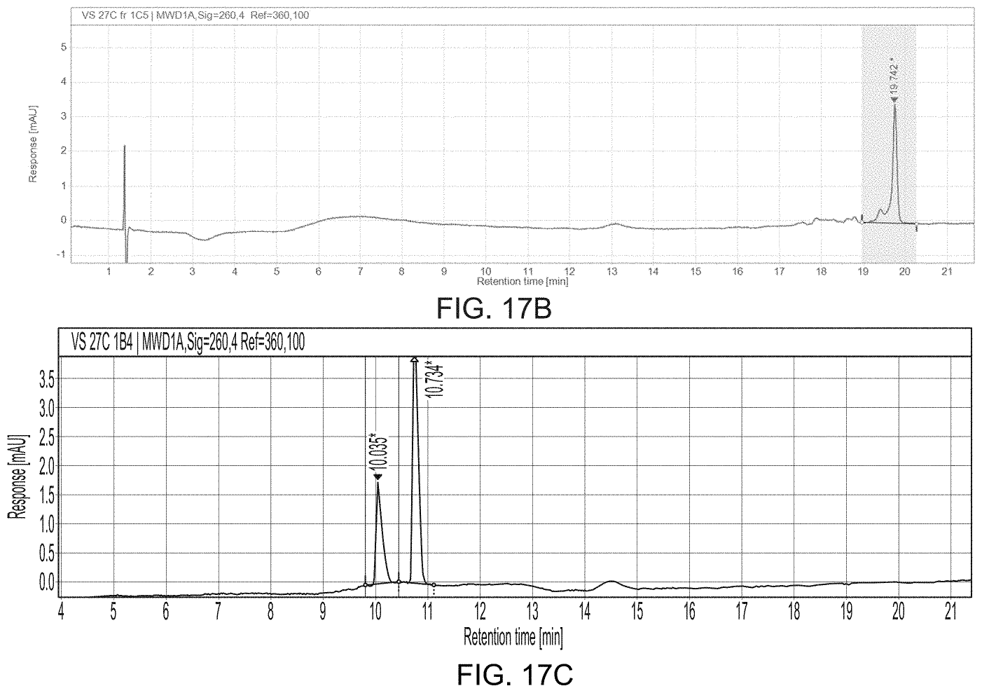

FIGS. 17A-C show HPLC separation of full length sgRNA with no DNA splints contamination observed (FIG. 17B) and DNA splints with no sgRNA observed (FIG. 17C).

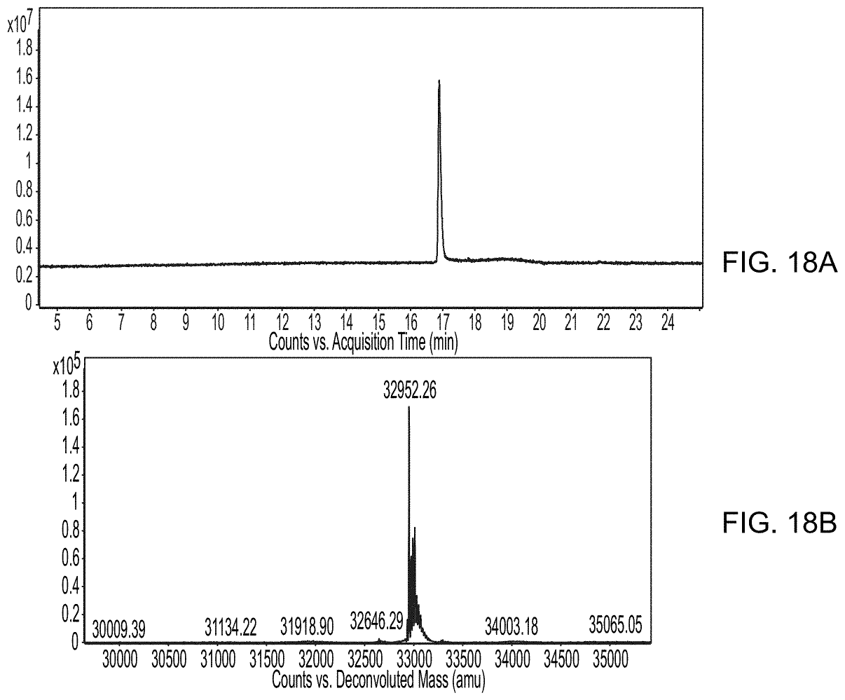

FIG. 18A shows LC/MC analysis of pure mT2-sgRNA and FIG. 18B shows the MS analysis of the single peak shown in FIG. 18A. The results demonstrated that very short splints mediated ligation can significantly improve the purity and yield of ligated free gRNA in large scale without the need of DNase treatment.

Additional gRNAs were also evaluated using short splints and very short splints. For example, hT5 and IL2RG gRNA synthesis were carried out by short splint- and very short-splint mediated ligation. hT5 RNA and IL2RG RNA were fragmented and modified (e.g., with 2′-O-methyl group and/or phosphorothioate linkage). In one set of experiments, hT5 RNA were fragmented into three RNA fragments: AUUUAUGAGAUCAACAGCACGUUUUAGAGCUAG (SEQ ID NO: 32), AAAUAGCAAGUUAAAAUAAGGCUAGUCCGUUAUCAACUU (SEQ ID NO: 33), and GAAAAAGUGGCACCGAGUCGGUGCUUUU (SEQ ID NO: 34). In another set of experiments, IL2RG RNA were fragmented into three RNA fragments: UGGUAAUGAUGGCUUCAACAGUUUUAGAGCUAG (SEQ ID NO: 35), AAAUAGCAAGUUAAAAUAAGGCUAGUCCGUUAUCAACUU (SEQ ID NO: 36), and GAAAAAGUGGCACCGAGUCGGUGCUUUU (SEQ ID NO: 37). Table 13 shows the above hT5 RNA and IL2RG fragments with modifications.

TABLE 13
Sequences of hT5 and IL2RG RNA fragments

| Oligos | Sequence |
|---|---|
| hT5 RNA1 | 5′-mA*mU*mU*rUrArUrGrArGrArUrCrArArCrArGr |
| | CrArCrGrUrUrUrUrArGrAmGmCmUmAmG-3′ |
| | (SEQ ID NO: 56) |
| RNA 2 | 5′-PaaauagcAAGUUAAAAUAAGGCUAGUCCGUUAUCaac |
| | uu-3′ (SEQ ID NO: 57) |
| RNA 3 | 5′-Pgaaaaaguggcaccgagucggugcusususu-3′ |
| | (SEQ ID NO: 58) |
| IL2RG | mU*mG*mG*UAAUGAUGGCUUCAACAGUUUUAGAGCUAG |
| RNA1 | (SEQ ID NO: 59) |
| RNA2 | PAAAUAGCAAGUUAAAAUAAGGCUAGUCCGUUAUCAACUU |
| | (SEQ ID NO: 60) |
| RNA3 | PGAAAAAGUGGCACCGAGUCGGUGCmU*mU*mU*U |
| | (SEQ ID NO: 61) |

mA, mC, mG, mU: 2′-O-methyl-nucleotide; a, g, u, c:
2′-O-methyl-nucleotide rA: riboadenosine; rG: riboguanosine;
rC: ribocytosine; rU: ribouridine or s = phosphorothioate
linkage; P: Phosphate

The ligation reaction mixture was prepared according to the procedure described above for mT2 RNA ligation preparation except that the hT5 and IL2RG RNA ligation reaction were carried out at 20 ml scale. Table 14 shows the IE gradient elution used for preparative IE-HPLC.

TABLE 14
IE gradient

| CV | % B |
|---|---|
| 1 | 0 |
| 2 | 0-30 |
| 15 | 30-55 |
| 1 | 100 |
| 1 | 0 |
| 1 | 100 |
| 3 | 0 |

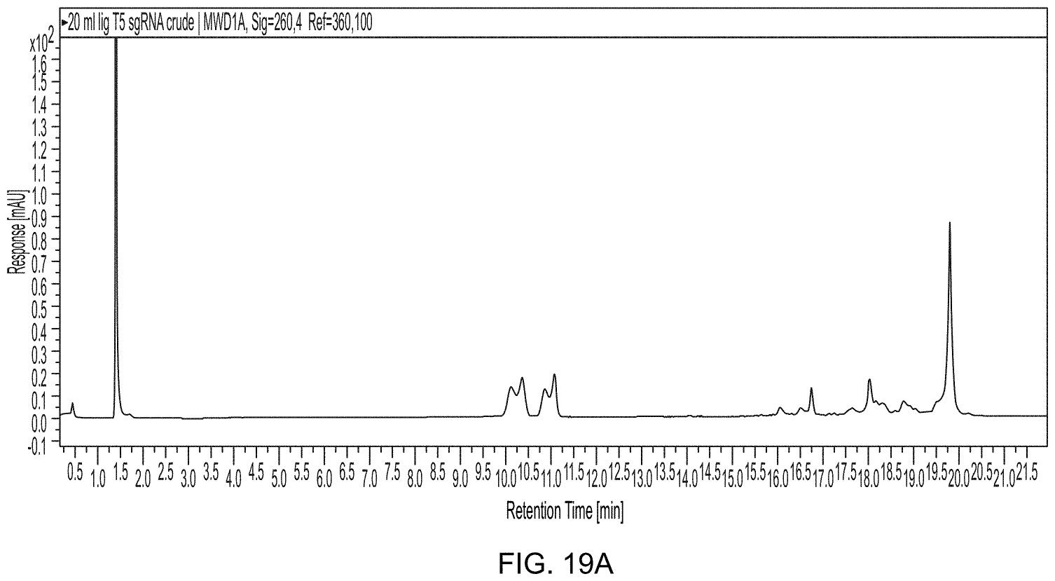

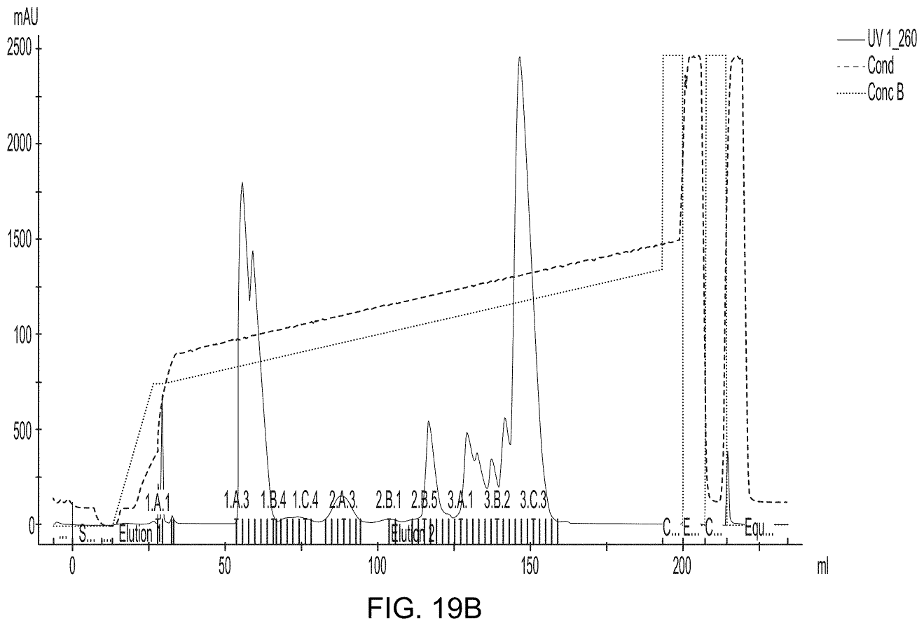

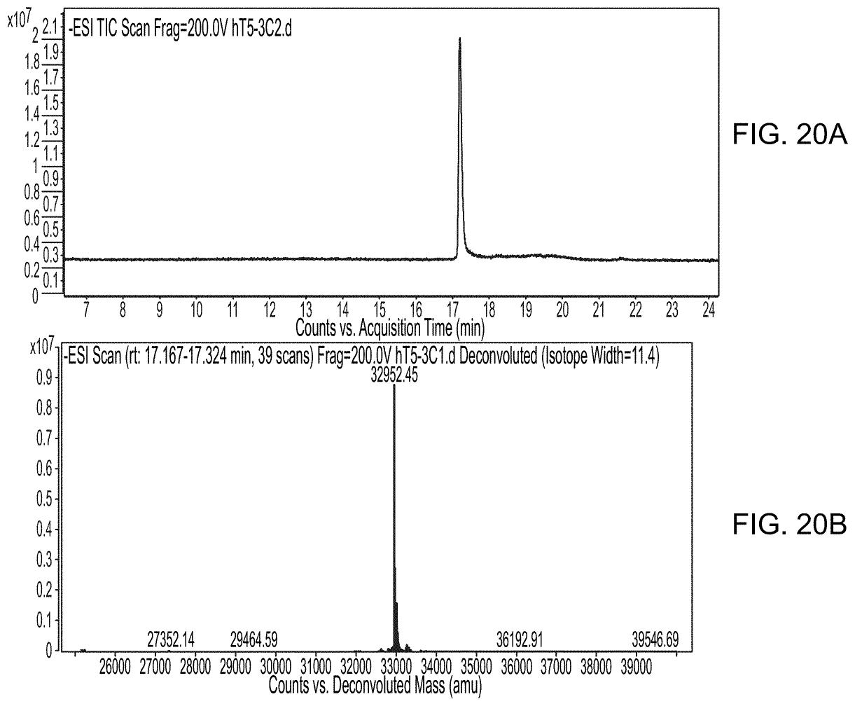

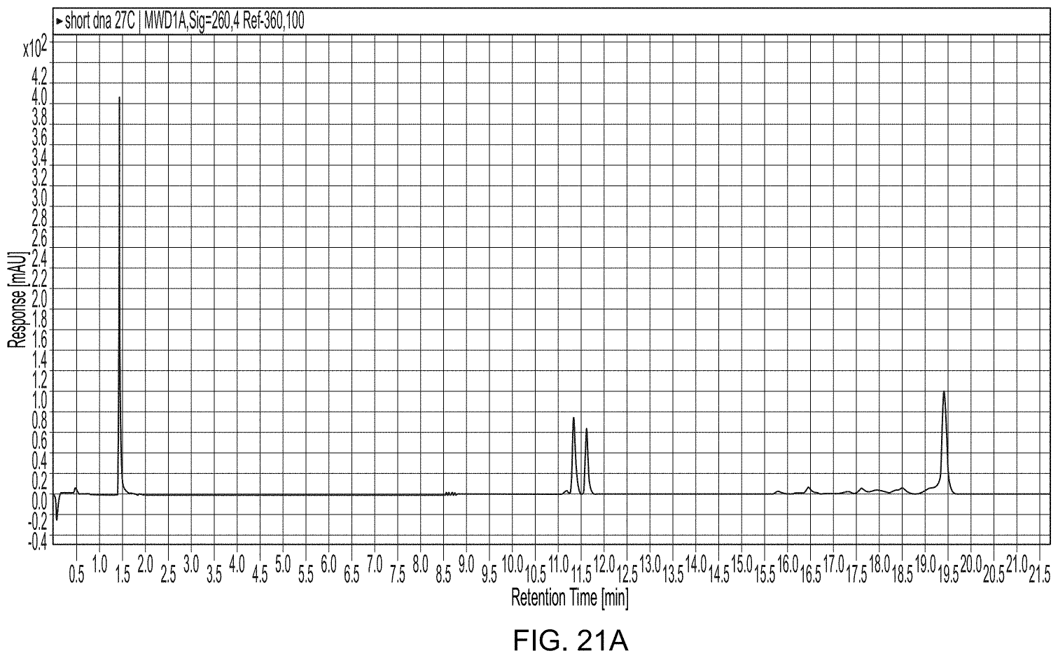

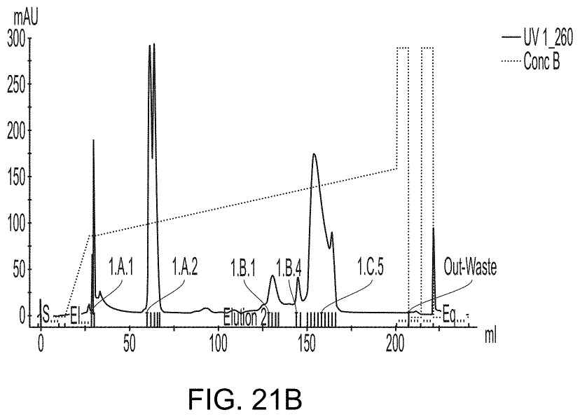

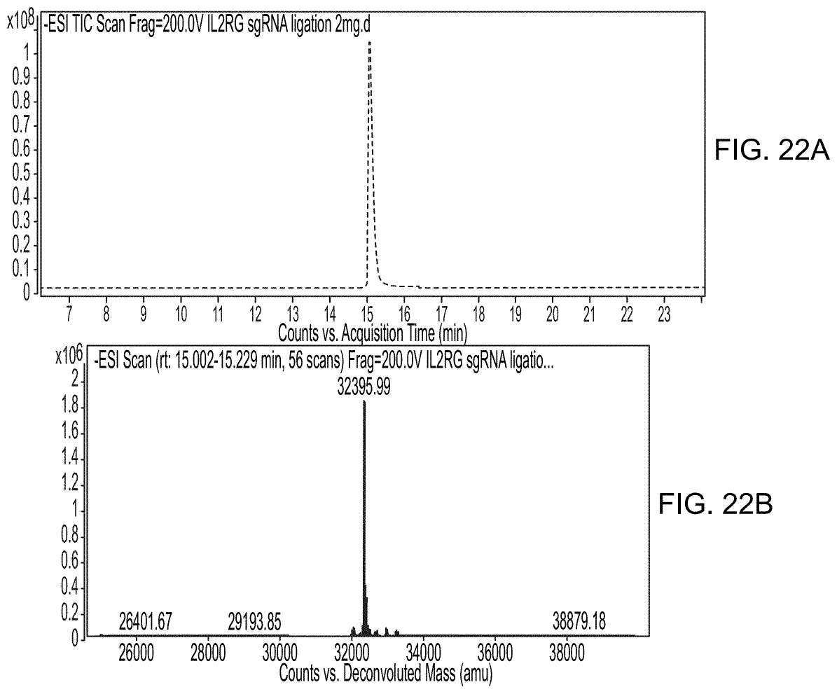

FIG. 19A shows HPLC of crude hT5 reaction. FIG. 19B shows HPLC purification of full length hT5 gRNA at 20 ml scale. FIGS. 20A-B show LC/MC analysis of pure hT5-sgRNA. FIG. 21A shows HPLC of crude IL2RG reaction. FIG. 21B shows HPLC purification of full length IL2RG gRNA at 20 ml scale. FIGS. 22A-B show LC/MC analysis of pure IL2RG-sgRNA. In house fragment analysis indicates a 88.3% purity of IL2RG gRNA. A 86% IL2RG gRNA purity was obtained with HR-UPLC.

The data demonstrates a successful purification of full length hT5 gRNA and IL2RG gRNA at 20 ml scale. The results further demonstrate that short splint- and very short splint-mediated gRNA synthesis can be applied to multiple targets with different backbone and chemistries.

### Example 6

### Short Splint- and Very Short Splint-Mediated Ligation at Low Temperatures

This example demonstrates that full-length gRNA molecules can be generated efficiently and in high purity using short splint- or very short splint-mediated ligation at low temperatures.

To further evaluate the possibility of lowering the reaction temperature, short splint and very short splint mediated ligation were conducted at 24° C., 22° C., 20° C. and 15° C. for 6 h according to the procedures previously described in Example 5.

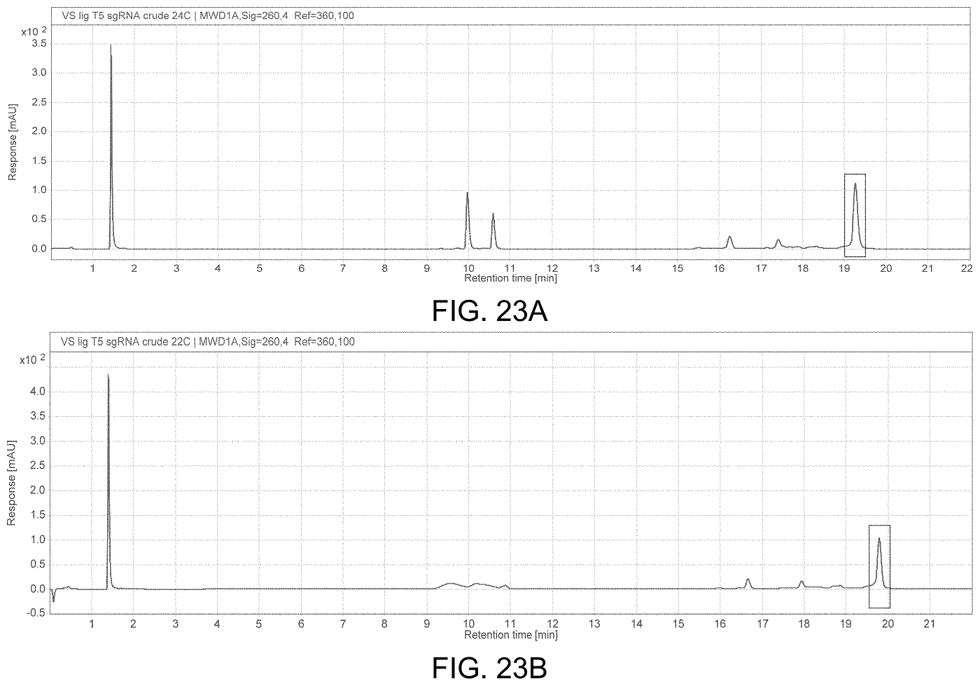

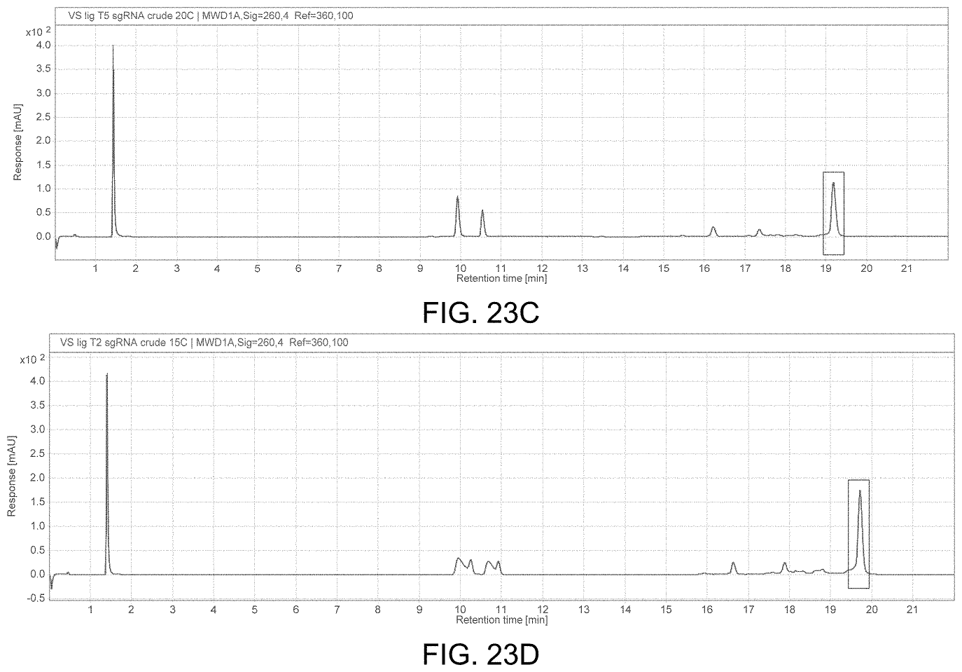

The pre-purified ligation components and the crude ligation reaction were analyzed by HPLC (FIGS. 23A-D). FIGS. 23A-D show purification by IE HPLC of crude of ligation reaction carried out at 24° C., 22° C., 20° C., and 15° C., respectively. The results showed that short and very short DNA splints can mediate a successful ligation reaction at a low temperature (e.g., 15° C.), thus making it possible to scale up the process.

### Example 7

### Ligase Amount Used in Splint-Mediated Ligation

For splint-mediated ligation reactions, 500 U ligase has typically been used for every ml reaction volume. Titration can be conducted to identify an optimal enzyme usage for a ligation reaction. This can be done by varying the amount of enzyme from 100-600 U/mL and analyzing by HPLC. HPLC analysis showed 200-300 U/ml ligase are sufficient to convert most materials into FLP. It has been demonstrated that both DNA splints do not get depleted and can be used to integrate as a constant to follow product formation kinetics. Table 15 below shows the components of an exemplary ligation reaction mixture.

TABLE 15
Components of an exemplary ligation mixture

| Component | Volume | |
|---|---|---|
| H₂O | 84-78 | μL |
| 10× Reaction Buffer (NEB #B0239S) | 10 | μL |
| DNA splint & RNA fragments (1 mM) | 1 | μL × 5 |
| T4 RNA Ligase 2 | 1-6 | μL (10-60 U) |
| Total | 100 | μL |

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of synthesizing a guide RNA (gRNA), the method comprising:
providing a first RNA fragment comprising a terminal region comprising a 5′ phosphate moiety, and a second RNA fragment comprising a terminal region comprising a 3′ hydroxyl group, wherein the first RNA fragment, the second RNA fragment, or both, comprises at least a portion of a sequence capable of binding to an RNA-guided endonuclease;
providing a splint DNA oligonucleotide comprising a first portion complementary to the first RNA fragment at the terminal region comprising a 5′ phosphate moiety and a second portion complementary to the second RNA fragment at the terminal region comprising a 3′ hydroxyl group;
hybridizing the first RNA fragment, the second RNA fragment, and the splint DNA oligonucleotide together to form a first complex;
contacting the first and second RNA fragments with a ligase to form a gRNA at a ligation site present between the first and second RNA fragments in the first complex to form a second complex, and
digesting the splint DNA oligonucleotide with a DNase to obtain the gRNA.

2. The method of claim 1, wherein digesting the splint DNA oligonucleotide with the DNase to obtain the gRNA comprises contacting the second complex with the DNase to digest the splint DNA oligonucleotide.

3. The method of claim 1, wherein digesting the splint DNA oligonucleotide with the DNase to obtain the gRNA comprises:
dissociating the gRNA and the splint DNA oligonucleotide; and
digesting the dissociated splint DNA oligonucleotide with the DNase to obtain gRNA.

4. The method of claim 1, wherein the DNase is a DNase I or a DNase II.

5. The method of claim 1, wherein the DNase is a bacterial DNase.

6. The method of claim 1, comprising separating the digested splint DNA oligonucleotide products from the gRNA.

7.-22. (canceled)

23. The method of claim 1, wherein the hybridizing a first RNA fragment, a second RNA fragment and a splint DNA oligonucleotide is carried out in the presence of one or more RNase inhibitors.

24. The method of claim 1, wherein the first RNA fragment, the second RNA fragment, or both, is about 10 to 90 nucleotides in length.

25. The method of claim 24, wherein the ratio between the length of the first RNA fragment and the length of the second RNA fragment is at least 20:80.

26. The method of claim 24, wherein the first RNA fragment is about 20 to 50 nucleotides in length, and the second RNA fragment is about 80 to 50 nucleotides in length.

27. The method of claim 1, wherein the 5′ phosphate moiety is 5′-phosphate or 5′-phosphorothioate.

28. The method of claim 1, wherein the ligase is a T4 DNA ligase, T4 RNA ligase I, or T4 RNA ligase II.

29. The method of claim 1, wherein the splint DNA oligonucleotide is 20 to 100 nucleotides in length.

30. The method of claim 1, wherein the splint DNA oligonucleotide is attached to a solid support.

31. The method of claim 1, wherein the ligation site corresponds to a site in a tetraloop portion of a stem-loop structure in the gRNA or a site in a helix portion of a stem-loop structure in the gRNA.

32. (canceled)

33. (canceled)

34. The method of claim 1, comprising ligating three or more RNA fragments.

35. (canceled)

36. (canceled)

37. (canceled)

38. (canceled)

39. (canceled)

40. The method of claim 1, wherein the first RNA fragment, the second RNA fragment, or both, comprises one or more modifications in the RNA backbone, one or more base modifications, one or more phosphorothioate linkages, or a combination thereof.

41.-60. (canceled)

61. A method of synthesizing a guide RNA (gRNA), the method comprising:
hybridizing a first RNA fragment, a second RNA fragment, a third RNA fragment, a first splint DNA oligonucleotide and a second first splint DNA oligonucleotide to form a complex, wherein
(a) the first RNA fragment comprises a terminal region comprising a 3′ hydroxyl group,
(b) the second RNA fragment comprises a first terminal region comprising a 5′ phosphate moiety and a second terminal region comprising a 3′ hydroxyl group,
(c) a third RNA fragment comprises a terminal region comprising a 5′ phosphate moiety,
(d) the first splint DNA oligonucleotide comprises (i) a first portion complementary to the terminal region comprising the 3′ hydroxyl group of the first RNA fragment; and (ii) a second portion complementary to the first terminal region comprising the 5′ phosphate moiety of the second RNA fragment;
(e) the second splint DNA oligonucleotide comprises (i) a first portion complementary to the second terminal region comprising the 3′ hydroxyl group of the second RNA fragment; and (ii) a second portion complementary to the terminal region comprising the 5′ phosphate moiety of the third RNA fragment,
wherein the complex comprises (i) a first ligation site present between the 3′ hydroxyl group of the first RNA fragment and the 5′ phosphate group of the second RNA fragment, and (i) a second ligation site present between the 3′ hydroxyl group of the second RNA fragment and the 5′ phosphate group of the third RNA fragment, and
wherein each of the first splint DNA oligonucleotide and the second splint DNA oligonucleotide is no more than 32 nucleotides in length; and
ligating the first and second RNA fragments, and the second and third RNA fragments, respectively, with a ligase at the first and second ligation sites in the complex, thereby synthesizing a gRNA.

62.-84. (canceled)

85. The method of claim 1, wherein the gRNA is a single gRNA (sgRNA).

86. A method of synthesizing a single guide RNA (sgRNA) for use with an RNA-guided endonuclease, comprising:
providing a first complex comprising a first RNA fragment, a second RNA fragment, a third RNA fragment, a first splint oligonucleotide, and a second splint oligonucleotide, wherein
(a) the first RNA fragment comprises (i) a terminal region comprising a 3′ hydroxyl group;
(b) the second RNA fragment comprises (i) a first terminal region comprising a 5′ phosphate moiety, and (ii) a second terminal region comprising a 3′ hydroxyl group;
(c) the third RNA fragment comprises (i) a terminal region comprising a 5′ phosphate moiety;
(d) the first splint oligonucleotide comprises (i) a first portion complementary to the terminal region comprising the 3′ hydroxyl group of the first RNA fragment, and (ii) a second portion complementary to the first terminal region comprising the 5′ phosphate moiety of the second RNA fragment; and
(e) the second splint oligonucleotide comprises (i) a first portion complementary to the second terminal region comprising the 3′ hydroxyl group of the second RNA fragment, and (ii) a second portion complementary to the terminal region comprising the 5′ phosphate moiety of the third RNA fragment,
wherein the first complex is formed by hybridization of (a)(i) and (d)(i), (b)(i) and (d)(ii), (b)(ii) and (e)(i), and (c)(i) and (e)(ii),
wherein the first complex has a first ligation site present between the 3′ hydroxyl group of the first RNA fragment and the 5′ phosphate group of the second RNA fragment, and a second ligation site present between the 3′ hydroxyl group of the second RNA fragment and the 5′ phosphate group of the third RNA fragment;
ligating the first RNA fragment and the second RNA fragment at the first ligation site and ligating the second RNA fragment and the third RNA fragment at the second ligation site to form a second complex comprising a sgRNA comprising from 5′ to 3′: a spacer sequence and an invariable sequence that binds an RNA-guided endonuclease; the invariable sequence comprising a stem loop formed between a crRNA repeat sequence and a tracrRNA anti-repeat sequence, and a 3′ tracrRNA sequence comprising at least one stem-loop; and
digesting the first and second splint DNA oligonucleotides with a DNase to obtain the sgRNA.

87.-128. (canceled)

129. The method of claim 1, wherein the gRNA is about 30 to about 160 nucleotides in length.

130.-149. (canceled)


SEQUENCE LISTING (72 sequences)

SEQ ID NO: 1 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 2 - RNA, 50 nt, synthetic construct
  misc_feature 1..50: note = Synthetic
  source 1..50: mol_type = other RNA
ctaacagttgcttttatcacgttttagagctagaaatagcaagttaaaat

SEQ ID NO: 3 - RNA, 50 nt, synthetic construct
  misc_feature 1..50: note = Synthetic
  source 1..50: mol_type = other RNA
aaggctagtccgttatcaacttgaaaaagtggcaccgagtcggtgctttt

SEQ ID NO: 4 - RNA, 40 nt, synthetic construct
  misc_feature 1..40: note = Synthetic
  source 1..40: mol_type = other RNA
ctaacagttgcttttatcacgttttagagctagaaatagc

SEQ ID NO: 5 - RNA, 60 nt, synthetic construct
  misc_feature 1..60: note = Synthetic
  source 1..60: mol_type = other RNA
aagttaaaataaggctagtccgttatcaacttgaaaaagtggcaccgagtcggtgctttt

SEQ ID NO: 6 - RNA, 35 nt, synthetic construct
  misc_feature 1..35: note = Synthetic
  source 1..35: mol_type = other RNA
ctaacagttgcttttatcacgttttagagctagaa

SEQ ID NO: 7 - RNA, 65 nt, synthetic construct
  misc_feature 1..65: note = Synthetic
  source 1..65: mol_type = other RNA
atagcaagttaaaataaggctagtccgttatcaacttgaaaaagtggcaccgagtcggtgctttt

SEQ ID NO: 8 - RNA, 50 nt, synthetic construct
  misc_feature 1..50: note = Synthetic
  source 1..50: mol_type = other RNA
gagaacgcaccactttacgagttttagagctagaaatagcaagttaaaat

SEQ ID NO: 9 - RNA, 50 nt, synthetic construct; identical to SEQ ID NO: 3
  misc_feature 1..50: note = Synthetic
  source 1..50: mol_type = other RNA

SEQ ID NO: 10 - RNA, 40 nt, synthetic construct
  misc_feature 1..40: note = Synthetic
  source 1..40: mol_type = other RNA
gagaacgcaccactttacgagttttagagctagaaatagc

SEQ ID NO: 11 - RNA, 60 nt, synthetic construct; identical to SEQ ID NO: 5
  misc_feature 1..60: note = Synthetic
  source 1..60: mol_type = other RNA

SEQ ID NO: 12 - RNA, 35 nt, synthetic construct
  misc_feature 1..35: note = Synthetic
  source 1..35: mol_type = other RNA
gagaacgcaccactttacgagttttagagctagaa

SEQ ID NO: 13 - RNA, 65 nt, synthetic construct; identical to SEQ ID NO: 7
  misc_feature 1..65: note = Synthetic
  source 1..65: mol_type = other RNA

SEQ ID NO: 14 - DNA, 33 bp, synthetic construct
  misc_feature 1..33: note = Synthetic
  source 1..33: mol_type = other DNA
cttattttaacttgctatttctagctctaaaac

SEQ ID NO: 15 - DNA, 36 bp, synthetic construct
  misc_feature 1..36: note = Synthetic
  source 1..36: mol_type = other DNA
ccttattttaacttgctatttctagctctaaaacgt

SEQ ID NO: 16 - DNA, 32 bp, synthetic construct
  misc_feature 1..32: note = Synthetic
  source 1..32: mol_type = other DNA
tagccttattttaacttgctatttctagctct

SEQ ID NO: 17 - DNA, 35 bp, synthetic construct
  misc_feature 1..35: note = Synthetic
  source 1..35: mol_type = other DNA
ctagccttattttaacttgctatttctagctctaa

SEQ ID NO: 18 - DNA, 30 bp, synthetic construct
  misc_feature 1..30: note = Synthetic
  source 1..30: mol_type = other DNA
cggactagccttattttaacttgctatttc

SEQ ID NO: 19 - DNA, 34 bp, synthetic construct
  misc_feature 1..34: note = Synthetic
  source 1..34: mol_type = other DNA
aacggactagccttattttaacttgctatttcta

SEQ ID NO: 20 - RNA, 33 nt, synthetic construct
  misc_feature 1..33: note = Synthetic
  source 1..33: mol_type = other RNA
gagaacgcaccactttacgagttttagagctag

SEQ ID NO: 21 - RNA, 39 nt, synthetic construct
  misc_feature 1..39: note = Synthetic
  source 1..39: mol_type = other RNA
aaatagcaagttaaaataaggctagtccgttatcaactt

SEQ ID NO: 22 - RNA, 28 nt, synthetic construct
  misc_feature 1..28: note = Synthetic
  source 1..28: mol_type = other RNA
gaaaaagtggcaccgagtcggtgctttt

SEQ ID NO: 23 - DNA, 36 bp, synthetic construct
  misc_feature 1..36: note = Synthetic
  source 1..36: mol_type = other DNA
ccttattttaacttgctatttctagctctaaaactc

SEQ ID NO: 24 - DNA, 48 bp, synthetic construct
  misc_feature 1..48: note = Synthetic
  source 1..48: mol_type = other DNA
aaaagcaccgactcggtgccactttttcaagttgataacggactagcc

SEQ ID NO: 25 - RNA, 33 nt, synthetic construct; identical to SEQ ID NO: 20
  misc_feature 1..33: note = Synthetic
  source 1..33: mol_type = other RNA

SEQ ID NO: 26 - RNA, 39 nt, synthetic construct; identical to SEQ ID NO: 21
  misc_feature 1..39: note = Synthetic
  source 1..39: mol_type = other RNA

SEQ ID NO: 27 - RNA, 28 nt, synthetic construct
  misc_feature 1..28: note = Synthetic
  source 1..28: mol_type = other RNA
gaaaaagtggcaccgagtcggtgctttt

SEQ ID NO: 28 - DNA, 32 bp, synthetic construct
  misc_feature 1..32: note = Synthetic
  source 1..32: mol_type = other DNA
tattttaacttgctatttctagctctaaaact

SEQ ID NO: 29 - DNA, 27 bp, synthetic construct
  misc_feature 1..27: note = Synthetic
  source 1..27: mol_type = other DNA
gtgccactttttcaagttgataacgga

SEQ ID NO: 30 - DNA, 26 bp, synthetic construct
  misc_feature 1..26: note = Synthetic
  source 1..26: mol_type = other DNA
ttttaacttgctatttctagctctaa

SEQ ID NO: 31 - DNA, 23 bp, synthetic construct
  misc_feature 1..23: note = Synthetic
  source 1..23: mol_type = other DNA
gccactttttcaagttgataacg

SEQ ID NO: 32 - RNA, 33 nt, synthetic construct
  misc_feature 1..33: note = Synthetic
  source 1..33: mol_type = other RNA
atttatgagatcaacagcacgttttagagctag

SEQ ID NO: 33 - RNA, 39 nt, synthetic construct; identical to SEQ ID NO: 21
  misc_feature 1..39: note = Synthetic
  source 1..39: mol_type = other RNA

SEQ ID NO: 34 - RNA, 28 nt, synthetic construct
  misc_feature 1..28: note = Synthetic
  source 1..28: mol_type = other RNA
gaaaaagtggcaccgagtcggtgctttt

SEQ ID NO: 35 - RNA, 33 nt, synthetic construct
  misc_feature 1..33: note = Synthetic
  source 1..33: mol_type = other RNA
tggtaatgatggcttcaacagttttagagctag

SEQ ID NO: 36 - RNA, 39 nt, synthetic construct; identical to SEQ ID NO: 21
  misc_feature 1..39: note = Synthetic
  source 1..39: mol_type = other RNA

SEQ ID NO: 37 - RNA, 28 nt, synthetic construct
  misc_feature 1..28: note = Synthetic
  source 1..28: mol_type = other RNA
gaaaaagtggcaccgagtcggtgctttt

SEQ ID NO: 38 - RNA, 50 nt, synthetic construct; identical to SEQ ID NO: 2
  source 1..50: mol_type = other RNA
  modified_base 1..3: mod_base = OTHER; note = 2'-O-methyl nucleotide
  misc_structure 1..4: note = Phosphorothioate linkage

SEQ ID NO: 39 - RNA, 50 nt, synthetic construct; identical to SEQ ID NO: 3
  source 1..50: mol_type = other RNA
  misc_feature 1: note = 5' is phosphate
  modified_base 47..49: mod_base = OTHER; note = 2'-O-methyl nucleotide
  misc_feature 47..50: note = Phosphorothioate linkage

SEQ ID NO: 40 - RNA, 40 nt, synthetic construct; identical to SEQ ID NO: 4
  source 1..40: mol_type = other RNA
  misc_feature 1..4: note = Phosphorothioate linkage
  modified_base 1..3: mod_base = OTHER; note = 2'-O-methyl nucleotide

SEQ ID NO: 41 - RNA, 60 nt, synthetic construct; identical to SEQ ID NO: 5
  source 1..60: mol_type = other RNA
  misc_feature 57..60: note = Phosphorothioate linkage
  modified_base 57..59: mod_base = OTHER; note = 2'-O-methyl nucleotide

SEQ ID NO: 42 - RNA, 35 nt, synthetic construct; identical to SEQ ID NO: 6
  source 1..35: mol_type = other RNA
  modified_base 1..3: mod_base = OTHER; note = 2'-O-methyl nucleotide
  misc_feature 1..4: note = Phosphorothioate linkage

SEQ ID NO: 43 - RNA, 65 nt, synthetic construct; identical to SEQ ID NO: 7
  source 1..65: mol_type = other RNA
  misc_feature 1: note = 5' is phosphate.
  modified_base 62..64: mod_base = OTHER; note = 2'-O-methyl nucleotide
  misc_feature 62..65: note = Phosphorothioate backbone linkage

SEQ ID NO: 44 - RNA, 50 nt, synthetic construct; identical to SEQ ID NO: 8
  source 1..50: mol_type = other RNA
  modified_base 1..3: mod_base = OTHER; note = 2'-O-methyl nucleotide
  misc_feature 1..4: note = Phosphorothioate backbone linkage
  modified_base 29..40: mod_base = OTHER; note = 2'-O-methyl nucleotide

SEQ ID NO: 45 - RNA, 50 nt, synthetic construct; identical to SEQ ID NO: 3
  source 1..50: mol_type = other RNA
  misc_feature 1: note = 5' is phosphate
  misc_feature 47..50: note = Phosphorothioate backbone linkage
  modified_base 19..50: mod_base = OTHER; note = 2'-O-methyl nucleotide

SEQ ID NO: 46 - RNA, 40 nt, synthetic construct; identical to SEQ ID NO: 10
  source 1..40: mol_type = other RNA
  modified_base 1..3: mod_base = OTHER; note = 2'-O-methyl nucleotide
  misc_feature 1..4: note = Phosphorothioate backbone linkage
  modified_base 29..40: mod_base = OTHER; note = 2'-O-methyl nucleotide

SEQ ID NO: 47 - RNA, 60 nt, synthetic construct; identical to SEQ ID NO: 5
  source 1..60: mol_type = other RNA
  misc_feature 1: note = 5' is phosphate
  misc_feature 57..60: note = Phosphorothioate backbone linkage
  modified_base 28..60: mod_base = OTHER; note = 2'-O-methyl nucleotide

SEQ ID NO: 48 - RNA, 35 nt, synthetic construct; identical to SEQ ID NO: 12
  source 1..35: mol_type = other RNA
  modified_base 1..3: mod_base = OTHER; note = 2'-O-methyl nucleotide
  misc_feature 1..4: note = Phosphorothioate backbone linkage
  modified_base 29..35: mod_base = OTHER; note = 2'-O-methyl nucleotide

SEQ ID NO: 49 - RNA, 65 nt, synthetic construct; identical to SEQ ID NO: 7
  source 1..65: mol_type = other RNA
  misc_feature 1: note = 5' is phosphate
  modified_base 1..5: mod_base = OTHER; note = 2'-O-methyl nucleotide
  modified_base 33..65: mod_base = OTHER; note = 2'-O-methyl nucleotide
  misc_feature 62..65: note = Phosphorothioate backbone linkage

SEQ ID NO: 50 - RNA, 33 nt, synthetic construct; identical to SEQ ID NO: 20
  source 1..33: mol_type = other RNA
  modified_base 1..3: mod_base = OTHER; note = 2'-O-methyl nucleotide
  misc_feature 1..4: note = Phosphorothioate backbone linkage
  modified_base 29..33: mod_base = OTHER; note = 2'-O-methyl nucleotide

SEQ ID NO: 51 - RNA, 39 nt, synthetic construct; identical to SEQ ID NO: 21
  source 1..39: mol_type = other RNA
  misc_feature 1: note = 5' is phosphate
  modified_base 1..7: mod_base = OTHER; note = 2'-O-methyl nucleotide
  modified_base 36..39: mod_base = OTHER; note = 2'-O-methyl nucleotide

SEQ ID NO: 52 - RNA, 28 nt, synthetic construct
  source 1..28: mol_type = other RNA
  misc_structure 1: note = 5' is phosphate
  modified_base 1..28: mod_base = OTHER; note = 2'-O-methyl nucleotide
  misc_feature 25..28: note = Phosphorothioate backbone linkage
gaaaaagtggcaccgagtcggtgctttt

SEQ ID NO: 53 - RNA, 33 nt, synthetic construct; identical to SEQ ID NO: 20
  source 1..33: mol_type = other RNA
  modified_base 1..3: mod_base = OTHER; note = 2'-O-methyl nucleotide
  misc_feature 1..4: note = phosphorothioate backbone linkage
  modified_base 29..33: mod_base = OTHER; note = 2'-O-methyl nucleotide

SEQ ID NO: 54 - RNA, 39 nt, synthetic construct; identical to SEQ ID NO: 21
  source 1..39: mol_type = other RNA
  misc_feature 1: note = 5' is a phosphate
  modified_base 1..7: mod_base = OTHER; note = 2'-O-methyl nucleotide
  modified_base 36..39: mod_base = OTHER; note = 2'-O-methyl nucleotide

SEQ ID NO: 55 - RNA, 28 nt, synthetic construct
  source 1..28: mol_type = other RNA
  misc_feature 1: note = 5' is a phosphate
  modified_base 1..28: mod_base = OTHER; note = 2'-O-methyl nucleotide
  misc_feature 25..28: note = phosphorothioate backbone linkage
gaaaaagtggcaccgagtcggtgctttt

SEQ ID NO: 56 - RNA, 33 nt, synthetic construct; identical to SEQ ID NO: 32
  source 1..33: mol_type = other RNA
  modified_base 1..3: mod_base = OTHER; note = 2'-O-methyl nucleotide
  misc_feature 1..4: note = phosphorothioate backbone linkage
  modified_base 29..33: mod_base = OTHER; note = 2'-O-methyl nucleotide

SEQ ID NO: 57 - RNA, 39 nt, synthetic construct; identical to SEQ ID NO: 21
  source 1..39: mol_type = other RNA
  misc_feature 1: note = 5' is attached to a phosphate
  modified_base 1..7: mod_base = OTHER; note = 2'-O-methyl nucleotide
  modified_base 35..39: mod_base = OTHER; note = 2'-O-methyl nucleotide

SEQ ID NO: 58 - RNA, 28 nt, synthetic construct
  source 1..28: mol_type = other RNA
  misc_feature 1: note = 5' is attached to a phosphate
  modified_base 1..28: mod_base = OTHER; note = 2'-O-methyl nucleotide
  misc_feature 25..28: note = phosphorothioate backbone linkage
gaaaaagtggcaccgagtcggtgctttt

SEQ ID NO: 59 - RNA, 33 nt, synthetic construct; identical to SEQ ID NO: 35
  source 1..33: mol_type = other RNA
  modified_base 1..3: mod_base = OTHER; note = 2'-O-methyl nucleotide
  misc_feature 1..4: note = phosphorothioate backbone linage

SEQ ID NO: 60 - RNA, 39 nt, synthetic construct; identical to SEQ ID NO: 21
  source 1..39: mol_type = other RNA
  misc_feature 1: note = 5' is attached to a phosphate

SEQ ID NO: 61 - RNA, 28 nt, synthetic construct
  source 1..28: mol_type = other RNA
  misc_feature 1: note = 5' is attached to a phosphate.
  modified_base 25..27: mod_base = OTHER; note = 2'-O-methyl nucleotide
  misc_feature 25..28: note = phosphorothioate backbone linkage
gaaaaagtggcaccgagtcggtgctttt

SEQ ID NO: 62 - RNA, 33 nt, synthetic construct
  source 1..33: mol_type = other RNA
  modified_base 1..3: mod_base = OTHER; note = 2'-O-methyl nucleotide
  misc_feature 1..4: note = Phosphorothioate backbone linkage
  modified_base 28..33: mod_base = OTHER; note = 2'-O-methyl nucleotide
tggacaacgcccgtattggtgttttagagctag

SEQ ID NO: 63 - RNA, 39 nt, synthetic construct; identical to SEQ ID NO: 21
  source 1..39: mol_type = other RNA
  misc_feature 1: note = 5' is attached to a phosphate.
  modified_base 1..7: mod_base = OTHER; note = 2'-O-methyl nucleotide
  modified_base 36..39: mod_base = OTHER; note = 2'-O-methyl nucleotide

SEQ ID NO: 64 - RNA, 28 nt, synthetic construct
  source 1..28: mol_type = other RNA
  misc_feature 1: note = 5'is attached to a phosphate.
  modified_base 1..28: mod_base = OTHER; note = 2'-O-methyl nucleotide
  misc_feature 25..28: note = Phosphorothioate backbone linkage
gaaaaagtggcaccgagtcggtgctttt

SEQ ID NO: 65 - RNA, 40 nt, synthetic construct
  source 1..40: mol_type = other RNA
  modified_base 1..3: mod_base = OTHER; note = N is 2'-O-methyl modified A, G, C or U.
  misc_feature 4..20: note = N is A, G, C or U.
  misc_feature 1..4: note = Phosphorothioate backbone linkage
  modified_base 29..40: mod_base = OTHER; note = 2'-O-methyl nucleotide
nnnnnnnnnnnnnnnnnnnngttttagagctagaaatagc

SEQ ID NO: 66 - RNA, 60 nt, synthetic construct; identical to SEQ ID NO: 5
  source 1..60: mol_type = other RNA
  misc_feature 1: note = 5' is attached to a phosphate.
  misc_feature 57..60: note = Phosphorothioate backbone linkage
  modified_base 28..60: mod_base = OTHER; note = 2'-O-methyl nucleotide

SEQ ID NO: 67 - RNA, 35 nt, synthetic construct
  source 1..35: mol_type = other RNA
  modified_base 1..3: mod_base = OTHER; note = N is 2'-O-methyl modified A, G, C or U.
  misc_feature 4..20: note = N is A, G, C or U.
  misc_feature 1..4: note = Phosphorothioate backbone linkage
  modified_base 29..35: mod_base = OTHER; note = 2'-O-methyl nucleotide
nnnnnnnnnnnnnnnnnnnngttttagagctagaa

SEQ ID NO: 68 - RNA, 65 nt, synthetic construct; identical to SEQ ID NO: 7
  source 1..65: mol_type = other RNA
  misc_feature 1: note = 5' is attached to a phosphate.
  modified_base 1..5: mod_base = OTHER; note = 2'-O-methyl nucleotide
  misc_feature 62..65: note = Phosphorothioate backbone linkage
  modified_base 33..65: mod_base = OTHER; note = 2'-O-methyl nucleotide

SEQ ID NO: 69 - RNA, 35 nt, synthetic construct; identical to SEQ ID NO: 67
  source 1..35: mol_type = other RNA
  misc_feature 1..20: note = N is A, G, C or U.

SEQ ID NO: 70 - RNA, 65 nt, synthetic construct; identical to SEQ ID NO: 7
  source 1..65: mol_type = other RNA
  misc_feature 1: note = 5' is attached to a phosphate.

SEQ ID NO: 71 - RNA, 40 nt, synthetic construct; identical to SEQ ID NO: 65
  source 1..40: mol_type = other RNA
  misc_feature 1..20: note = N is A, G, C or U.

SEQ ID NO: 72 - skipped